(19) United States
(12) Patent Application Publication
(10) Pub. No.: US 2025/0136574 A1
(43) Pub. Date: May 1, 2025

(54) SELECTIVE AGENTS TARGETING MYCOBACTERIUM TUBERCULOSIS

(71) Applicants: UNIVERSITY OF MISSISSIPPI, University, MS (US); WASHINGTON UNIVERSITY, St. Louis, MO (US); UNIVERSITÄT DES SAARLANDES, Saarbrücken (DE)

(72) Inventors: Sudeshna Roy, Oxford, MS (US); Robert Doerksen, Oxford, MS (US); Tomayo Berida, Oxford, MS (US); Christina Stallings, St. Louis, MO (US); Samuel McKee, Creve Coeur, MO (US); Christian Ducho, Saarbrücken (DE)

(21) Appl. No.: 18/833,489

(22) PCT Filed: Jan. 31, 2023
(86) PCT No.: PCT/US2023/061619

§ 371 Date: Jul. 26, 2024

Related U.S. Application Data
(60) Provisional application No. 63/267,357, filed on Jan. 31, 2022.

(51) Int. Cl. C07D 401/04; A61K 31/4196; A61K 31/4439; A61P 31/06; C07D 249/12; C07D 401/14; C07D 403/04; C07D 405/14; C07D 413/14

(52) U.S. Cl. CPC C07D 401/04; A61K 31/4196; A61K 31/4439; A61P 31/06; C07D 249/12; C07D 401/14; C07D 403/04; C07D 405/14; C07D 413/14

(57) ABSTRACT

In one aspect, the disclosure relates to compounds effective in the treatment of multidrug-resistant tuberculosis and extensively drug-resistant tuberculosis as well as drug-sensitive tuberculosis, methods of making the same, pharmaceutical compositions comprising the same, and methods of treating bacterial infections caused by Mycobacterium tuberculosis using the same. In an aspect, the compounds and pharmaceutical compositions are not cytotoxic. This abstract is intended as a scanning tool for purposes of searching in the particular art and is not intended to be limiting of the present disclosure.

### CROSS-REFERENCE TO RELATED APPLICATIONS

This application claims the benefit of U.S. Provisional Application No. 63/267,357, filed on Jan. 31, 2022, which is incorporated herein by reference in its entirety.

### STATEMENT REGARDING FEDERALLY SPONSORED RESEARCH OR DEVELOPMENT

This invention was made with government support under grant number R21 AI142210 awarded by the National Institute of Allergy and Infectious Diseases of the National Institutes of Health. The government has certain rights in the invention.

### BACKGROUND

Tuberculosis (TB), an airborne infectious disease caused by Mycobacterium tuberculosis (Mtb), was responsible for 1.4 million deaths in 2019, making it a major global health threat.

Although tuberculosis is curable using combination therapy including the four first line agents rifampicin, isoniazid, ethambutol, and pyrazinamide, the development of antibiotic resistance poses a major threat to control of the disease.

Furthermore, resistance to rifampicin and isoniazid, referred to as multidrug-resistant TB (MDR-TB), is rapidly increasing. In 2019, the World Health Organization (WHO) reported an estimated 206,030 cases of MDR-TB or rifampicin-resistant TB (RR-TB) worldwide, amounting to a 10% increase from the 186,883 cases reported in 2018. The development of resistance to these agents is blamed on the general mismanagement of antibiotics and the long course of treatment required to successfully treat MDR-TB, which results in an increased risk of side effects and is often accompanied by poor patient compliance.

Thus, treatment of MDR-TB requires the addition of second line drugs, including a fluoroquinolone, along with any of the injectable aminoglycosides such as amikacin, streptomycin, and/or kanamycin. Regrettably, this addition is proving insufficient in many cases due to the emergence of extensively drug-resistant tuberculosis (XDR-TB). XDR-TB strains typically have resistance to three or more first-line drugs as well as second-line anti-TB injectable drugs. The recent approval of pretomanid in combination with bedaquiline and delamanid obviously strengthened the TB armamentarium. However, this combination is reserved for only few cases and resistant strains for bedaquiline and delamanid have already been isolated.

Despite advances in tuberculosis research, there is still a scarcity of compounds that are effective in the treatment of MDR-TB and XDR-TB while also requiring a relatively short course of treatment that results in few side effects, resulting in higher levels of patient compliance and emergence of few or no additional drug-resistant TB strains. Such new treatments for TB would, ideally, be useful in both drug-sensitive and drug-resistant cases. These needs and other needs are satisfied by the present disclosure.

### SUMMARY

In accordance with the purpose(s) of the present disclosure, as embodied and broadly described herein, the disclosure, in one aspect, relates to compounds effective in the treatment of multidrug-resistant tuberculosis and extensively drug-resistant tuberculosis as well as drug-sensitive tuberculosis, methods of making the same, pharmaceutical compositions comprising the same, and methods of treating bacterial infections caused by Mycobacterium tuberculosis using the same. In an aspect, the compounds and pharmaceutical compositions are not cytotoxic.

Other systems, methods, features, and advantages of the present disclosure will be or become apparent to one with skill in the art upon examination of the following drawings and detailed description. It is intended that all such additional systems, methods, features, and advantages be included within this description, be within the scope of the present disclosure, and be protected by the accompanying claims. In addition, all optional and preferred features and modifications of the described embodiments are usable in all aspects of the disclosure taught herein. Furthermore, the individual features of the dependent claims, as well as all optional and preferred features and modifications of the described embodiments are combinable and interchangeable with one another.

### DETAILED DESCRIPTION

In one aspect, disclosed herein are compounds of Formula Ia or Ib,

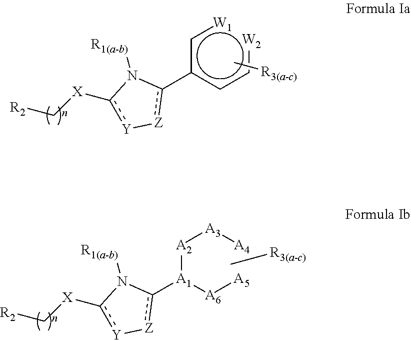

wherein W₁ and W₂ independently are N or CR₁₀;
wherein X is S, O, —NH, N—R₄, or CH₂;
wherein Y and Z independently are S, O, N, NH, N—R₄, CH, or CH₂;
   wherein R₄ is hydrogen, halogen, hydroxyl, cyano, nitro, carboxyl, (carboxyl ester)amino, (carboxyl ester)oxy, acyl, formyl; or substituted or unsubstituted C1-C10 alkyl, C3-C7 cycloalkyl, C3-C7 heterocycloalkyl, alkenyl, alkoxy, alkynyl, amino, aminosulfinyl, sulfinyl, sulfonyl, substituted sulfinyloxy, sulfonyloxy, aminosulfonyloxy, aminosulfinyloxy, aminosulfonylamino, acylamino, aminocarbonyloxy, aminocarbonylamino, aminothiocarbonylamino, aminocarbonyl, aminocarbonyl, acyloxy, C3-C7 aryl, or C3-C7 heteroaryl;
wherein n is from 0 to 9;
wherein each of R₁ₐ and R₂ is independently hydrogen, halogen, hydroxyl, cyano, nitro, carboxyl, (carboxyl ester)amino, (carboxyl ester)oxy, acyl, formyl; or substituted or unsubstituted C1-C10 alkyl, C3-C7 cycloalkyl, C3-C7 heterocycloalkyl, alkenyl, alkoxy, alkynyl, amino, aminosulfinyl, sulfinyl, sulfonyl, substituted sulfinyloxy, sulfonyloxy, aminosulfonyloxy, aminosulfinyloxy, aminosulfonylamino, acylamino, aminocarbonyloxy, aminocarbonylamino, aminothiocarbonylamino, aminocarbonyl, aminocarbonyl, acyloxy, C3-C7 aryl, or C3-C7 heteroaryl;
wherein each of R_{3a-3c} is independently hydrogen, halogen, hydroxyl, cyano, nitro, carboxyl, (carboxyl ester)amino, (carboxyl ester)oxy, acyl, formyl; or substituted or unsubstituted C1-C10 alkyl, C3-C7 cycloalkyl, C3-C7 heterocycloalkyl, alkenyl, alkoxy, alkynyl, amino, aminosulfinyl, sulfinyl, sulfonyl, substituted sulfinyloxy, sulfonyloxy, aminosulfonyloxy, aminosulfinyloxy, aminosulfonylamino, acylamino, aminocarbonyloxy, aminocarbonylamino, aminothiocarbonylamino, aminocarbonyl, aminocarbonyl, acyloxy, C3-C7 aryl, or C3-C7 heteroaryl, or wherein one of R_{3a-3c} is an N, NR₁₁, CH, or CH₂ group connected to an R₁₀ by a substituted or unsubstituted C1-C3 alkyl chain;
wherein each R₁₀ is independently H or is an N, NR₁₂, CH, or CH₂ group connected to one of R_{3a-3c} by a substituted or unsubstituted C1-C3 alkyl chain;
wherein R₁₁ and R₁₂, if present, independently are H or C1-C4 alkyl;
wherein R_{1b} is absent or is hydrogen, halogen, hydroxyl, cyano, nitro, carboxyl, (carboxyl ester)amino, (carboxyl ester)oxy, acyl, formyl; or substituted or unsubstituted C1-C10 alkyl, C3-C7 cycloalkyl, C3-C7 heterocycloalkyl, alkenyl, alkoxy, alkynyl, amino, aminosulfinyl, sulfinyl, sulfonyl, substituted sulfinyloxy, sulfonyloxy, aminosulfonyloxy, aminosulfinyloxy, aminosulfonylamino, acylamino, aminocarbonyloxy, aminocarbonylamino, aminothiocarbonylamino, aminocarbonyl, aminocarbonyl, acyloxy, C3-C7 aryl, or C3-C7 heteroaryl; and
wherein A₁-A₆ are independently selected from NR₁₄, CR₁₄, C(R₁₄)₂, N, and O, wherein each R₁₄ is independently H or is one of R_{3a-3c}.

In some aspects, W₁ is N and W₂ is CR₁₀, and R₁₀ is H. In another aspect, the compound has a formula

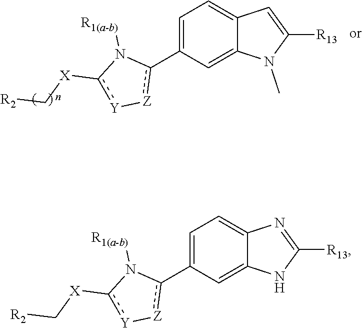

or, wherein R₁₃ is selected from hydrogen, halogen, hydroxyl, cyano, nitro, carboxyl, (carboxyl ester)amino, (carboxyl ester)oxy, acyl, formyl; or substituted or unsubstituted C1-C10 alkyl, C3-C7 cycloalkyl, C3-C7 heterocycloalkyl, alkenyl, alkoxy, alkynyl, amino, aminosulfinyl, sulfinyl, sulfonyl, substituted sulfinyloxy, sulfonyloxy, aminosulfonyloxy, aminosulfinyloxy, aminosulfonylamino, acylamino, aminocarbonyloxy, aminocarbonylamino, aminothiocarbonylamino, aminocarbonyl, aminocarbonyl, acyloxy, C3-C7 aryl, or C3-C7 heteroaryl In another aspect, Y and Z are N and R_{1b} is absent. In an alternative aspect, Y and Z are CH and R_{1b} is absent. In an aspect, R₁ₐ is methyl, ethyl, cyclopropyl, or —CFH₂. In some aspects, R₁₃ can be

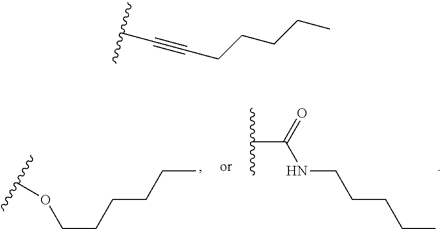

In another aspect, R₂ is

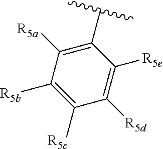

and R₅ₐ-R₅ₑ are independently hydrogen, halogen, cyano, or nitro. In some aspects, R_{5b} and R_{5d} are nitro and R₅ₐ, R_{5c}, and R₅ₑ are hydrogen. In other aspects, R₅ₐ, R_{5b}, R_{5d}, and R₅ₑ, are hydrogen and R_{5c} is chloro or bromo. In still another aspect, R₅ₐ, R_{5d}, and R₅ₑ, are hydrogen, R_{5b} is chloro, and R_{5c} is chloro or fluoro.

In one aspect, R₂ is

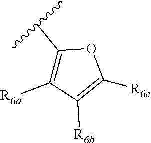

and R₆ₐ-R_{6c} are independently hydrogen or nitro. In a further aspect, R_{6c} is nitro and R₆ₐ and R_{6b} are hydrogen.

In any of these aspects, n can be 0 or 1. In one aspect, X is O. In another aspect, X is S.

In one aspect, A₁-A₆ and R_{3a-3c}, if present, can form a saturated heterocycle selected from:

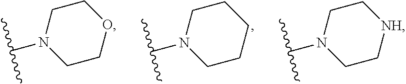 [SMILES: *N1CCOCC1 ; *N1CCCCC1 ; "," ; *N1CCNCC1]

or any combination thereof.

In one aspect, the compound is

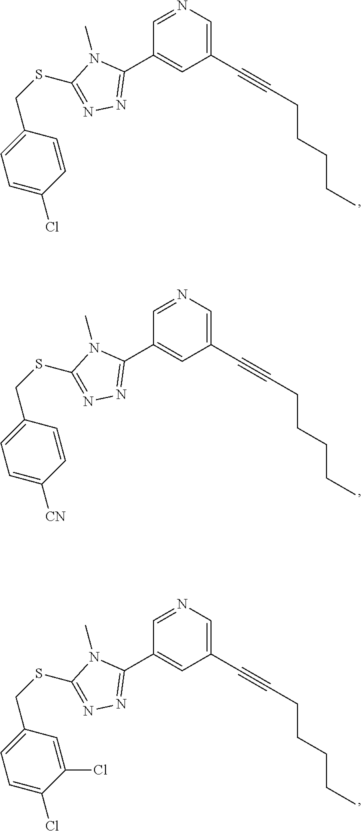 [SMILES: CCCCCC#Cc1cncc(-c2nnc(SCc3ccc(Cl)cc3)n2C)c1 ; CCCCCC#Cc1cncc(-c2nnc(SCc3ccc(C#N)cc3)n2C)c1 ; CCCCCC#Cc1cncc(-c2nnc(SCc3ccc(Cl)c(Cl)c3)n2C)c1]

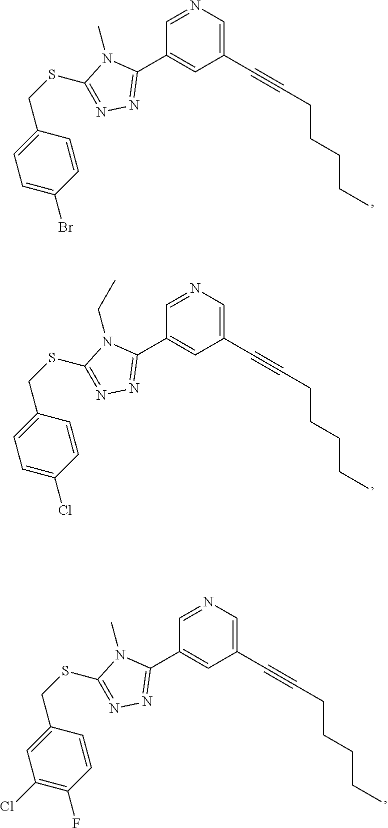 [SMILES: CCCCCC#Cc1cncc(-c2nnc(SCc3ccc(Br)cc3)n2C)c1 ; CCCCCC#Cc1cncc(-c2nnc(SCc3ccc(Cl)cc3)n2CC)c1 ; CCCCCC#Cc1cncc(-c2nnc(SCc3ccc(F)c(Cl)c3)n2C)c1]

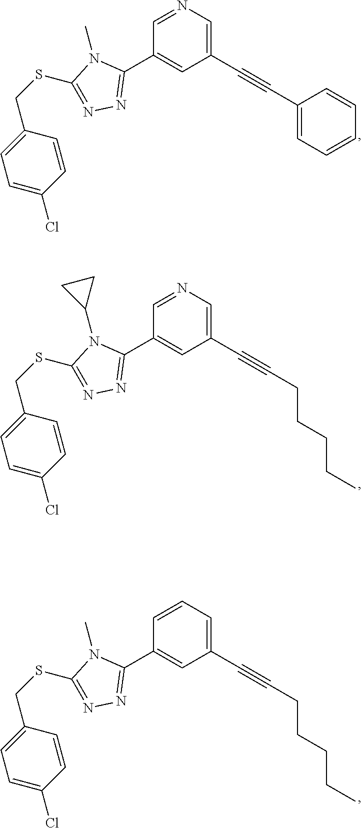 [SMILES: Cn1c(SCc2ccc(Cl)cc2)nnc1-c1cncc(C#Cc2ccccc2)c1 ; CCCCCC#Cc1cncc(-c2nnc(SCc3ccc(Cl)cc3)n2C2CC2)c1 ; CCCCCC#Cc1cccc(-c2nnc(SCc3ccc(Cl)cc3)n2C)c1]

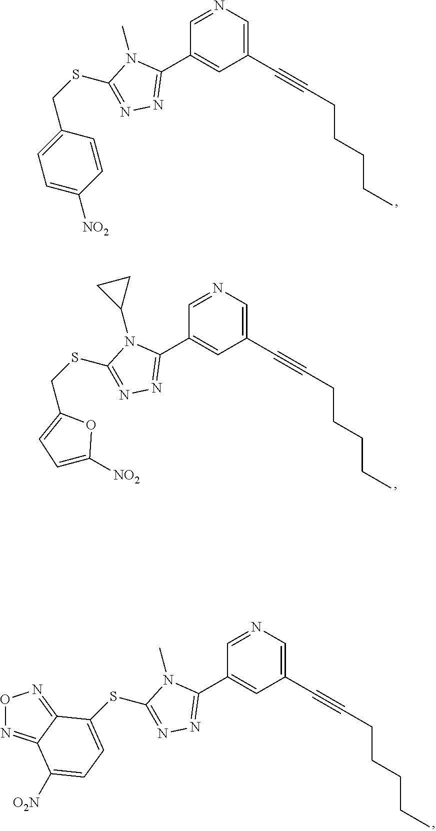 [SMILES: CCCCCC#Cc1cncc(-c2nnc(SCc3ccc([N+](=O)[O-])cc3)n2C)c1 ; CCCCCC#Cc1cncc(-c2nnc(SCc3ccc([N+](=O)[O-])o3)n2C2CC2)c1 ; CCCCCC#Cc1cncc(-c2nnc(Sc3ccc([N+](=O)[O-])c4nonc34)n2C)c1]

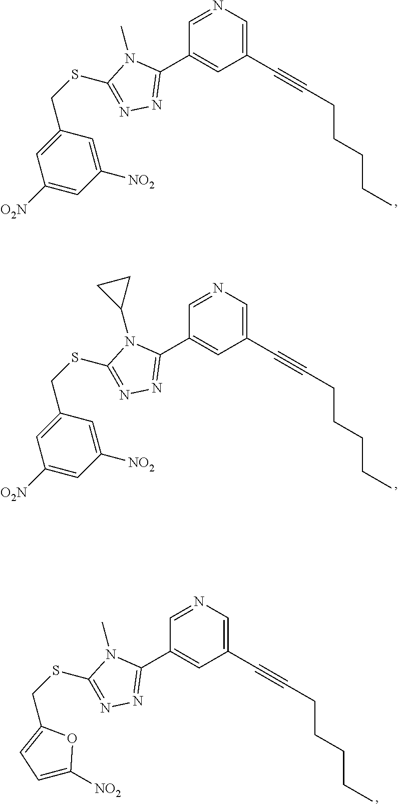 [SMILES: CCCCCC#Cc1cncc(-c2nnc(SCc3cc([N+](=O)[O-])cc([N+](=O)[O-])c3)n2C)c1 ; CCCCCC#Cc1cncc(-c2nnc(SCc3cc([N+](=O)[O-])cc([N+](=O)[O-])c3)n2C2CC2)c1 ; CCCCCC#Cc1cncc(-c2nnc(SCc3ccc([N+](=O)[O-])o3)n2C)c1]

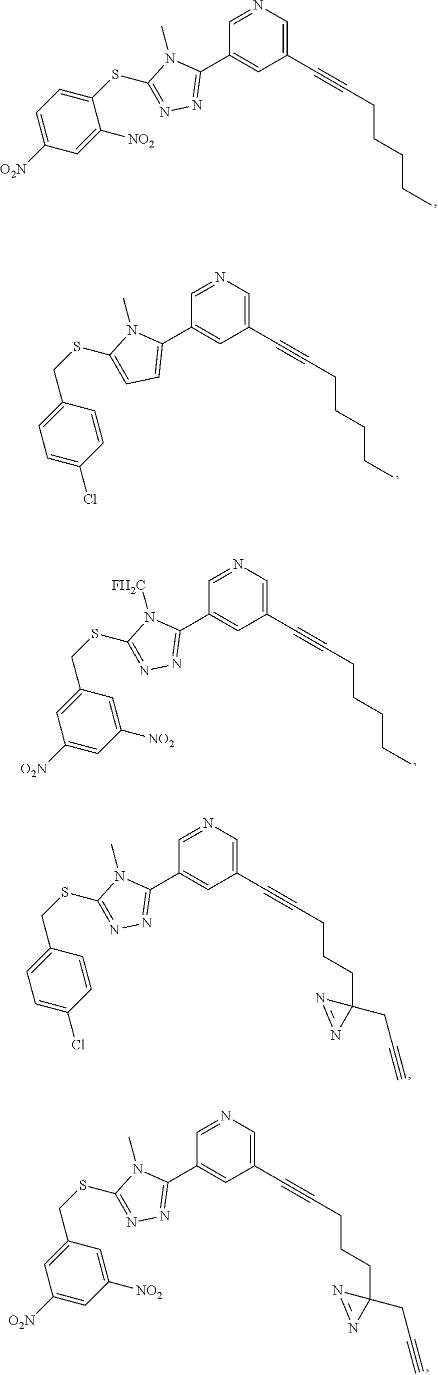

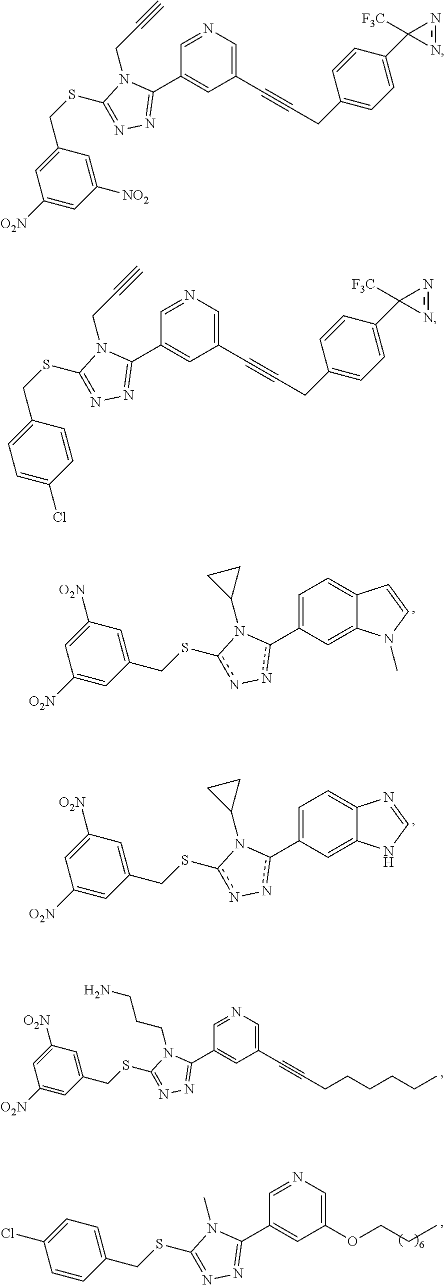

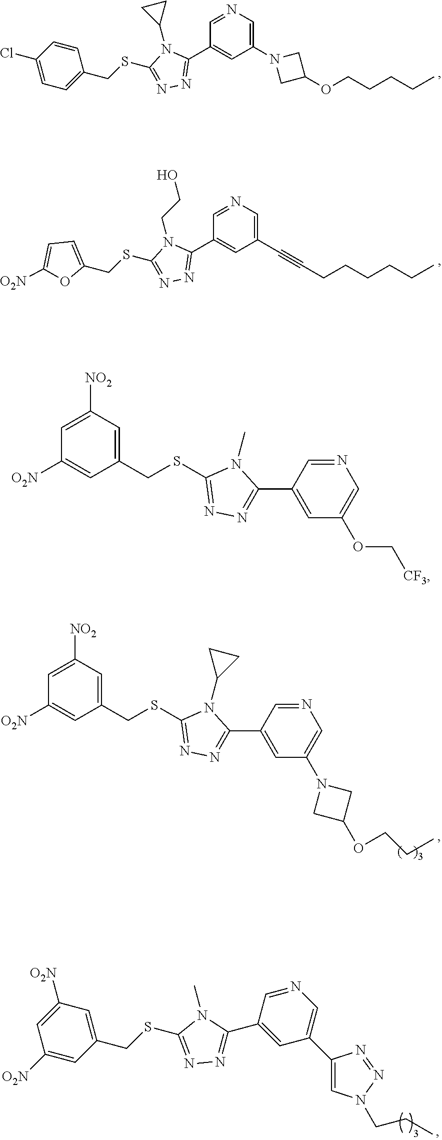

a salt thereof, or any combination thereof.

Also disclosed are compounds of Formula II,

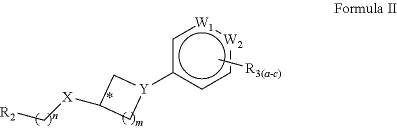

wherein W₁ and W₂ independently are N or CR₁₀;
wherein X is S, O, —N—H, N—R₄, or CH₂;
wherein Y is N, NR₉, or CH;
wherein m and n are independently from 0 to 9; and
wherein each of R₂ and R₉, if present, is independently hydrogen, halogen, hydroxyl, cyano, nitro, carboxyl, (carboxyl ester)amino, (carboxyl ester)oxy, acyl, formyl; or substituted or unsubstituted C1-C10 alkyl, C3-C7 cycloalkyl, C3-C7 heterocycloalkyl, alkenyl, alkoxy, alkynyl, amino, aminosulfinyl, sulfinyl, sulfonyl, substituted sulfinyloxy, sulfonyloxy, aminosulfonyloxy, aminosulfinyloxy, aminosulfonylamino, acylamino, aminocarbonyloxy, aminocarbonylamino, aminothiocarbonylamino, aminocarbonyl, aminocarbonyl, acyloxy, C3-C7 aryl, or C3-C7 heteroaryl;
wherein each of R_{3a-3c} is independently hydrogen, halogen, hydroxyl, cyano, nitro, carboxyl, (carboxyl ester)amino, (carboxyl ester)oxy, acyl, formyl; or substituted or unsubstituted C1-C10 alkyl, C3-C7 cycloalkyl, C3-C7 heterocycloalkyl, alkenyl, alkoxy, alkynyl, amino, aminosulfinyl, sulfinyl, sulfonyl, substituted sulfinyloxy, sulfonyloxy, aminosulfonyloxy, aminosulfinyloxy, aminosulfonylamino, acylamino, aminocarbonyloxy, aminocarbonylamino, aminothiocarbonylamino, aminocarbonyl, aminocarbonyl, acyloxy, C3-C7 aryl, or C3-C7 heteroaryl, or wherein one of R_{3a-3c} is an N, NR₁₁, CH, or CH₂ group connected to an R₁₀ by a substituted or unsubstituted C1-C3 alkyl chain;
wherein each R₁₀ independently is H or is an N, NR₁₂, CH, or CH₂ group connected to one of R_{3a-3c} by a substituted or unsubstituted C1-C3 alkyl chain; and
wherein R₁₁ and R₁₂, if present, independently are H or C1-C4 alkyl.

In one aspect, in the compound of Formula II, W₁ is N and W₂ is CR₁₀, wherein R₁₀ is H. In another aspect, Y is N. In one aspect, X is O. In an alternative aspect, X is S.

In an aspect m is 1 or 2. In one aspect, the compound has a stereochemistry of (R) or (S) at a carbon indicated by *.

In any of these aspects, R₂ can be

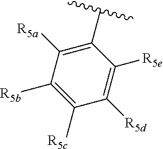

and R₅ₐ-R₅ₑ are independently hydrogen, halogen, cyano, or nitro. In one aspect, R_{5b} and R_{5d} are nitro and R₅ₐ, R_{5c}, and R₅ₑ are hydrogen. In another aspect, R₅ₐ, R_{5b}, R_{5d}, and R₅ₑ, are hydrogen and R_{5c} is chloro.

In any of these aspects, n is 0 or 1.

In one aspect the compound is

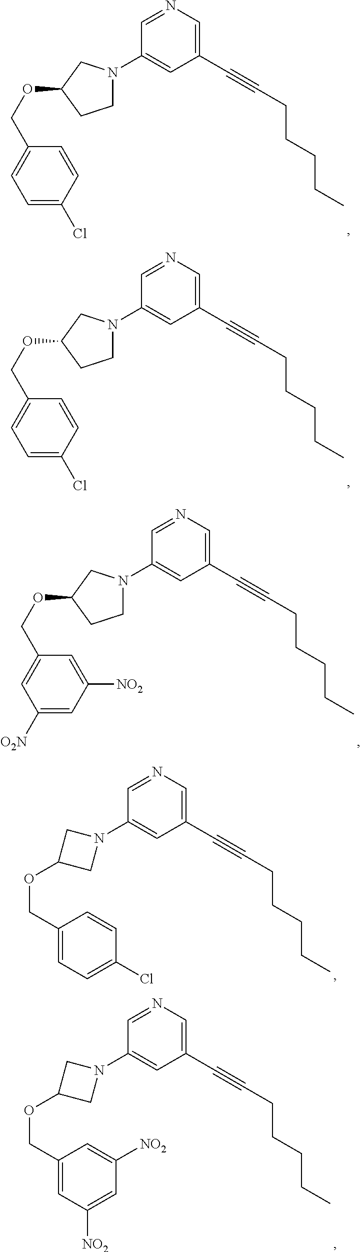

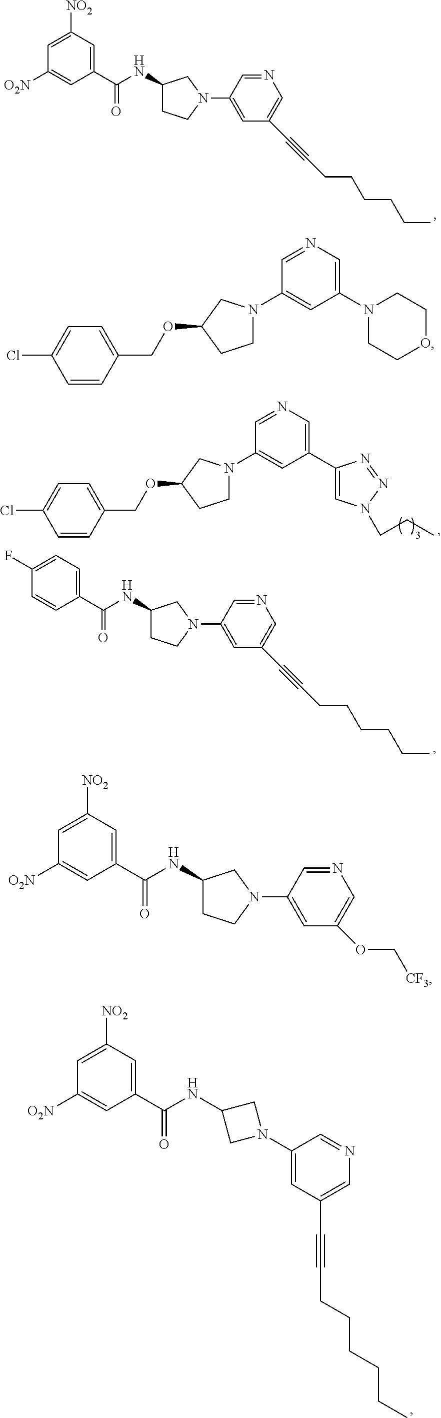

a salt thereof, or any combination thereof.

Also disclosed are compounds of Formula III,

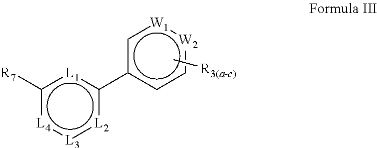

wherein W₁ and W₂ independently are N or CH;
wherein each of L₁, L₂, L₃, and L₄ independently is N or CH, or, optionally, one of L₁, L₂, L₃, or L₄ independently comprises CR₈;
   wherein R₇, or, if present, R₈, is

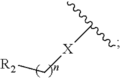

wherein X is S, O, —N—H, N—R₄, or CH₂;
wherein n is from 0 to 9; and
wherein each of R₂ and R_{3a-3c} is hydrogen, halogen, hydroxyl, cyano, nitro, carboxyl, (carboxyl ester)amino, (carboxyl ester)oxy, acyl, formyl; or substituted or unsubstituted C1-C10 alkyl, C3-C7 cycloalkyl, C3-C7 heterocycloalkyl, alkenyl, alkoxy, alkynyl, amino, aminosulfinyl, sulfinyl, sulfonyl, substituted sulfinyloxy, sulfonyloxy, aminosulfonyloxy, aminosulfinyloxy, aminosulfonylamino, acylamino, aminocarbonyloxy, aminocarbonylamino, aminothiocarbonylamino, aminocarbonyl, aminocarbonyl, acyloxy, C3-C7 aryl, or C3-C7 heteroaryl.

In one aspect, in the compound of Formula III, W₁ is N and W₂ is CH. In another aspect, R₂ is

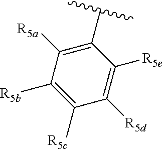

and R₅ₐ-R₅ₑ are independently hydrogen, halogen, cyano, or nitro. In a further aspect, R_{5b} and R_{5d} are nitro and R₅ₐ, R_{5c}, and R₅ₑ are hydrogen, or R₅ₐ, R_{5b}, R₅ₐ, and R₅ₑ, are hydrogen and R_{5c} is chloro.

In one aspect, n is 0 or 1. In a further aspect, X is O. In an alternative aspect, X is S. In any of these aspects, L₁, L₂, and L₄ can be CH and L₃ can be N.

In one aspect, the compound of Formula III is

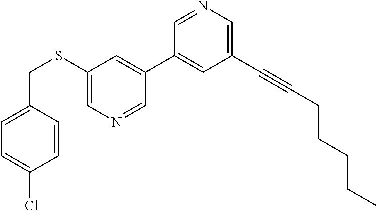 [SMILES: CCCCCC#Cc1cncc(-c2cncc(SCc3ccc(Cl)cc3)c2)c1]

or a salt thereof.

In any of these aspects, the salt of Formula Ia, Ib, II, or III can be a hydrochloride salt.

Also disclosed are pharmaceutical compositions containing the compounds and methods of treating bacterial infections caused by Mtb in a subject, wherein the methods include the step of administering a disclosed compound or pharmaceutical composition to the subject.

In one aspect, the disclosed compound or pharmaceutical composition can have an IC₅₀ of less than about 1 μM against Mycobacterium tuberculosis, or of less than about 80, 60, or 50 μM, or a combination of any of the foregoing values, or a range encompassing any of the foregoing values.

In another aspect, the compound or pharmaceutical composition can exhibit an MraY inhibition of at least about 45% at a 250 μM concentration. In a further aspect, the compound or pharmaceutical composition is not cytotoxic. In some aspects, cytotoxicity of the disclosed compounds and pharmaceutical compositions can be assessed by calculating a 50% cytotoxic concentration (CC₅₀, or the concentration that results in a 50% decrease in cellular ATP) for the compound. In a further aspect, following determination of CC₅₀, a selectivity index (SI) can be calculated by determining the ratio of CC₅₀ in a given cell type to IC₅₀ in Mtb. In a still further aspect, the higher the SI, the safer the compound is in eukaryotic cells relative to its effective dose. SI values for selected compounds are provided in Table 6 in the Examples. In one aspect, SI can be determined in Vero cells as a representative cell type useful for modeling mammalian cytotoxicity.

In yet another aspect, the compound can be useful for photoaffinity labeling. Further in this aspect, the compound can contain one or more reactive alkynyl and/or diazirine groups for the purpose of reaction with a target substrate, wherein reaction binds the compound to the substrate for the purpose of visualizing using a method such as, for example, fluorescence microscopy, fluorescence spectroscopy, or the like.

Many modifications and other embodiments disclosed herein will come to mind to one skilled in the art to which the disclosed compositions and methods pertain having the benefit of the teachings presented in the foregoing descriptions and the associated drawings. Therefore, it is to be understood that the disclosures are not to be limited to the specific embodiments disclosed and that modifications and other embodiments are intended to be included within the scope of the appended claims. The skilled artisan will recognize many variants and adaptations of the aspects described herein. These variants and adaptations are intended to be included in the teachings of this disclosure and to be encompassed by the claims herein.

Although specific terms are employed herein, they are used in a generic and descriptive sense only and not for purposes of limitation.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure.

Any recited method can be carried out in the order of events recited or in any other order that is logically possible. That is, unless otherwise expressly stated, it is in no way intended that any method or aspect set forth herein be construed as requiring that its steps be performed in a specific order. Accordingly, where a method claim does not specifically state in the claims or descriptions that the steps are to be limited to a specific order, it is in no way intended that an order be inferred, in any respect. This holds for any possible non-express basis for interpretation, including matters of logic with respect to arrangement of steps or operational flow, plain meaning derived from grammatical organization or punctuation, or the number or type of aspects described in the specification.

All publications mentioned herein are incorporated herein by reference to disclose and describe the methods and/or materials in connection with which the publications are cited. The publications discussed herein are provided solely for their disclosure prior to the filing date of the present application. Nothing herein is to be construed as an admission that the present invention is not entitled to antedate such publication by virtue of prior invention. Further, the dates of publication provided herein can be different from the actual publication dates, which can require independent confirmation.

While aspects of the present disclosure can be described and claimed in a particular statutory class, such as the system statutory class, this is for convenience only and one of skill in the art will understand that each aspect of the present disclosure can be described and claimed in any statutory class.

It is also to be understood that the terminology used herein is for the purpose of describing particular aspects only and is not intended to be limiting. Unless defined otherwise, all technical and scientific terms used herein have the same meaning as commonly understood by one of ordinary skill in the art to which the disclosed compositions and methods belong. It will be further understood that terms, such as those defined in commonly used dictionaries, should be interpreted as having a meaning that is consistent with their meaning in the context of the specification and relevant art and should not be interpreted in an idealized or overly formal sense unless expressly defined herein.

Prior to describing the various aspects of the present disclosure, the following definitions are provided and should be used unless otherwise indicated. Additional terms may be defined elsewhere in the present disclosure.

### Definitions

As used herein, “comprising” is to be interpreted as specifying the presence of the stated features, integers, steps, or components as referred to, but does not preclude the presence or addition of one or more features, integers, steps, or components, or groups thereof. Moreover, each of the terms “by,” “comprising,” “comprises,” “comprised of,” “including,” “includes,” “included,” “involving,” “involves,” “involved,” and “such as” are used in their open, non-limiting sense and may be used interchangeably. Further, the term “comprising” is intended to include examples and aspects encompassed by the terms “consisting essentially of” and “consisting of.” Similarly, the term “consisting essentially of” is intended to include examples encompassed by the term “consisting of.”

As used in the specification and the appended claims, the singular forms “a,” “an” and “the” include plural referents unless the context clearly dictates otherwise. Thus, for example, reference to “a leaving group,” “a base,” or “an aryl group,” include, but are not limited to, mixtures or combinations of two or more such leaving groups, bases, or aryl groups, and the like.

It should be noted that ratios, concentrations, amounts, and other numerical data can be expressed herein in a range format. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. Ranges can be expressed herein as from “about” one particular value, and/or to “about” another particular value. Similarly, when values are expressed as approximations, by use of the antecedent “about,” it will be understood that the particular value forms a further aspect. For example, if the value “about 10” is disclosed, then “10” is also disclosed.

When a range is expressed, a further aspect includes from the one particular value and/or to the other particular value. For example, where the stated range includes one or both of the limits, ranges excluding either or both of those included limits are also included in the disclosure, e.g. the phrase “x to y” includes the range from ‘x’ to ‘y’ as well as the range greater than ‘x’ and less than ‘y.’ The range can also be expressed as an upper limit, e.g. ‘about x, y, z, or less’ and should be interpreted to include the specific ranges of ‘about x,’ ‘about y,’ and ‘about z’ as well as the ranges of ‘less than x,’ ‘less than y,’ and ‘less than z.’ Likewise, the phrase ‘about x, y, z, or greater’ should be interpreted to include the specific ranges of ‘about x,’ ‘about y,’ and ‘about z’ as well as the ranges of ‘greater than x,’ ‘greater than y,’ and ‘greater than z.’ In addition, the phrase “about ‘x’ to ‘y’”, where ‘x’ and ‘y’ are numerical values, includes “about ‘x’ to about ‘y’”.

It is to be understood that such a range format is used for convenience and brevity, and thus, should be interpreted in a flexible manner to include not only the numerical values explicitly recited as the limits of the range, but also to include all the individual numerical values or sub-ranges encompassed within that range as if each numerical value and sub-range is explicitly recited. To illustrate, a numerical range of “about 0.1% to 5%” should be interpreted to include not only the explicitly recited values of about 0.1% to about 5%, but also include individual values (e.g., about 1%, about 2%, about 3%, and about 4%) and the sub-ranges (e.g., about 0.5% to about 1.1%; about 5% to about 2.4%; about 0.5% to about 3.2%, and about 0.5% to about 4.4%, and other possible sub-ranges) within the indicated range.

As used herein, the terms “about,” “approximate,” “at or about,” and “substantially” mean that the amount or value in question can be the exact value or a value that provides equivalent results or effects as recited in the claims or taught herein. That is, it is understood that amounts, sizes, formulations, parameters, and other quantities and characteristics are not and need not be exact, but may be approximate and/or larger or smaller, as desired, reflecting tolerances, conversion factors, rounding off, measurement error and the like, and other factors known to those of skill in the art such that equivalent results or effects are obtained. In some circumstances, the value that provides equivalent results or effects cannot be reasonably determined. In such cases, it is generally understood, as used herein, that “about” and “at or about” mean the nominal value indicated ±10% variation unless otherwise indicated or inferred. In general, an amount, size, formulation, parameter or other quantity or characteristic is “about,” “approximate,” or “at or about” whether or not expressly stated to be such. It is understood that where “about,” “approximate,” or “at or about” is used before a quantitative value, the parameter also includes the specific quantitative value itself, unless specifically stated otherwise.

As used herein, the terms “optional” or “optionally” means that the subsequently described event or circumstance can or cannot occur, and that the description includes instances where said event or circumstance occurs and instances where it does not.

Unless otherwise specified, pressures referred to herein are based on atmospheric pressure (i.e. one atmosphere).

As used herein, “IC₅₀,” is intended to refer to the concentration of a substance (e.g., a compound or a drug) that is required for 50% inhibition of a biological process, or component of a process. For example, IC₅₀ refers to the half maximal (50%) inhibitory concentration (IC) of a substance as determined in a suitable assay. For example, an IC₅₀ in Mtb can be determined in an in vitro or cell-based assay system. In some aspects, % inhibition at a particular concentration (e.g. 100 μM or 250 μM) can instead be calculated using a Microplate Alamar Blue Assay (MABA) as described in the Examples.

As used herein, “cytotoxic” refers to a substance, compound, or composition, that results in cell damage and/or cell death. In one aspect, a compound safe for therapeutic use is not cytotoxic to a patient's cells; i.e., the compound will act against an infectious agent such as, for example, Mycobacterium tuberculosis, and/or against a protein target (e.g., MraY in Mtb) without harming the subject to whom the compound is administered.

A residue of a chemical species, as used in the specification and concluding claims, refers to the moiety that is the resulting product of the chemical species in a particular reaction scheme or subsequent formulation or chemical product, regardless of whether the moiety is actually obtained from the chemical species. Thus, an ethylene glycol residue in a polyester refers to one or more —OCH₂CH₂O— units in the polyester, regardless of whether ethylene glycol was used to prepare the polyester. Similarly, a sebacic acid residue in a polyester refers to one or more —CO(CH₂)₈CO— moieties in the polyester, regardless of whether the residue is obtained by reacting sebacic acid or an ester thereof to obtain the polyester.

As used herein, the term “substituted” is contemplated to include all permissible substituents of organic compounds. In a broad aspect, the permissible substituents include acyclic and cyclic, branched and unbranched, carbocyclic and heterocyclic, and aromatic and nonaromatic substituents of organic compounds. Illustrative substituents include, for example, those described below. The permissible substituents can be one or more and the same or different for appropriate organic compounds. For purposes of this disclosure, the heteroatoms, such as nitrogen, can have hydrogen substituents and/or any permissible substituents of organic compounds described herein which satisfy the valences of the heteroatoms. This disclosure is not intended to be limited in any manner by the permissible substituents of organic compounds.

Also, the terms “substitution” or “substituted with” include the implicit proviso that such substitution is in accordance with permitted valence of the substituted atom and the substituent, and that the substitution results in a stable compound, e.g., a compound that does not spontaneously undergo transformation such as by rearrangement, cyclization, elimination, etc. It is also contemplated that, in certain aspects, unless expressly indicated to the contrary, individual substituents can be further optionally substituted (i.e., further substituted or unsubstituted).

In defining various terms, “A¹,” “A²,” “A³,” and “A⁴” are used herein as generic symbols to represent various specific substituents. These symbols can be any substituent, not limited to those disclosed herein, and when they are defined to be certain substituents in one instance, they can, in another instance, be defined as some other substituents.

The term “aliphatic” or “aliphatic group,” as used herein, denotes a hydrocarbon moiety that may be straight-chain (i.e., unbranched), branched, or cyclic (including fused, bridging, and spirofused polycyclic) and may be completely saturated or may contain one or more units of unsaturation, but which is not aromatic. Unless otherwise specified, aliphatic groups contain 1-20 carbon atoms. Aliphatic groups include, but are not limited to, linear or branched, alkyl, alkenyl, and alkynyl groups, and hybrids thereof such as (cycloalkyl)alkyl, (cycloalkenyl)alkyl or (cycloalkyl)alkenyl.

The term “alkyl” as used herein is a branched or unbranched saturated hydrocarbon group of 1 to 24 carbon atoms, such as methyl, ethyl, n-propyl, isopropyl, n-butyl, isobutyl, s-butyl, t-butyl, n-pentyl, isopentyl, s-pentyl, neopentyl, hexyl, heptyl, octyl, nonyl, decyl, dodecyl, tetradecyl, hexadecyl, eicosyl, tetracosyl, and the like. The alkyl group can be cyclic or acyclic.

The alkyl group can be branched or unbranched. The alkyl group can also be substituted or unsubstituted. For example, the alkyl group can be substituted with one or more groups including, but not limited to, alkyl, cycloalkyl, alkoxy, amino, ether, halide, hydroxy, nitro, silyl, sulfo-oxo, or thiol, as described herein. A “lower alkyl” group is an alkyl group containing from one to six (e.g., from one to four) carbon atoms. The term alkyl group can also be a C1 alkyl, C1-C2 alkyl, C1-C3 alkyl, C1-C4 alkyl, C1-C5 alkyl, C1-C6 alkyl, C1-C7 alkyl, C1-C8 alkyl, C1-C9 alkyl, C1-C10 alkyl, and the like up to and including a C1-C24 alkyl.

Throughout the specification “alkyl” is generally used to refer to both unsubstituted alkyl groups and substituted alkyl groups; however, substituted alkyl groups are also specifically referred to herein by identifying the specific substituent(s) on the alkyl group. For example, the term “halogenated alkyl” or “haloalkyl” specifically refers to an alkyl group that is substituted with one or more halide, e.g., fluorine, chlorine, bromine, or iodine. Alternatively, the term “monohaloalkyl” specifically refers to an alkyl group that is substituted with a single halide, e.g. fluorine, chlorine, bromine, or iodine. The term “polyhaloalkyl” specifically refers to an alkyl group that is independently substituted with two or more halides, i.e. each halide substituent need not be the same halide as another halide substituent, nor do the multiple instances of a halide substituent need to be on the same carbon. The term “alkoxyalkyl” specifically refers to an alkyl group that is substituted with one or more alkoxy groups, as described below. The term “aminoalkyl” specifically refers to an alkyl group that is substituted with one or more amino groups. The term “hydroxyalkyl” specifically refers to an alkyl group that is substituted with one or more hydroxy groups. When “alkyl” is used in one instance and a specific term such as “hydroxyalkyl” is used in another, it is not meant to imply that the term “alkyl” does not also refer to specific terms such as “hydroxyalkyl” and the like.

This practice is also used for other groups described herein. That is, while a term such as “cycloalkyl” refers to both unsubstituted and substituted cycloalkyl moieties, the substituted moieties can, in addition, be specifically identified herein; for example, a particular substituted cycloalkyl can be referred to as, e.g., an “alkylcycloalkyl.” Similarly, a substituted alkoxy can be specifically referred to as, e.g., a “halogenated alkoxy,” a particular substituted alkenyl can be, e.g., an “alkenylalcohol,” and the like. Again, the practice of using a general term, such as “cycloalkyl,” and a specific term, such as “alkylcycloalkyl,” is not meant to imply that the general term does not also include the specific term.

The term “cycloalkyl” as used herein is a non-aromatic carbon-based ring composed of at least three carbon atoms. Examples of cycloalkyl groups include, but are not limited to, cyclopropyl, cyclobutyl, cyclopentyl, cyclohexyl, norbornyl, and the like. The term “heterocycloalkyl” is a type of cycloalkyl group as defined above, and is included within the meaning of the term “cycloalkyl,” where at least one of the carbon atoms of the ring is replaced with a heteroatom such as, but not limited to, nitrogen, oxygen, sulfur, or phosphorus. The cycloalkyl group and heterocycloalkyl group can be substituted or unsubstituted. The cycloalkyl group and heterocycloalkyl group can be substituted with one or more groups including, but not limited to, alkyl, cycloalkyl, alkoxy, amino, ether, halide, hydroxy, nitro, silyl, sulfo-oxo, or thiol as described herein.

The term “alkanediyl” as used herein, refers to a divalent saturated aliphatic group, with one or two saturated carbon atom(s) as the point(s) of attachment, a linear or branched, cyclo, cyclic or acyclic structure, no carbon-carbon double or triple bonds, and no atoms other than carbon and hydrogen. The groups, —CH₂— (methylene), —CH₂CH₂—, —CH₂C(CH₃)₂CH₂—, and —CH₂CH₂CH₂— are non-limiting examples of alkanediyl groups.

The terms “alkoxy” and “alkoxyl” as used herein to refer to an alkyl or cycloalkyl group bonded through an ether linkage; that is, an “alkoxy” group can be defined as —OA¹ where A¹ is alkyl or cycloalkyl as defined above. “Alkoxy” also includes polymers of alkoxy groups as just described; that is, an alkoxy can be a polyether such as —OA¹-OA² or —OA¹-(OA²)ₐ-OA³, where “a” is an integer of from 1 to 200 and A¹, A², and A³ are alkyl and/or cycloalkyl groups.

The term “alkenyl” as used herein is a hydrocarbon group of from 2 to 24 carbon atoms with a structural formula containing at least one carbon-carbon double bond. Asymmetric structures such as (A¹A²)C═C(A³A⁴) are intended to include both the E and Z isomers. This can be presumed in structural formulae herein wherein an asymmetric alkene is present, or it can be explicitly indicated by the bond symbol C═C. The alkenyl group can be substituted with one or more groups including, but not limited to, alkyl, cycloalkyl, alkoxy, alkenyl, cycloalkenyl, alkynyl, cycloalkynyl, aryl, heteroaryl, aldehyde, amino, carboxylic acid, ester, ether, halide, hydroxy, ketone, azide, nitro, silyl, sulfo-oxo, or thiol, as described herein.

The term “cycloalkenyl” as used herein is a non-aromatic carbon-based ring composed of at least three carbon atoms and containing at least one carbon-carbon double bound, i.e., C═C. Examples of cycloalkenyl groups include, but are not limited to, cyclopropenyl, cyclobutenyl, cyclopentenyl, cyclopentadienyl, cyclohexenyl, cyclohexadienyl, norbornenyl, and the like. The term “heterocycloalkenyl” is a type of cycloalkenyl group as defined above, and is included within the meaning of the term “cycloalkenyl,” where at least one of the carbon atoms of the ring is replaced with a heteroatom such as, but not limited to, nitrogen, oxygen, sulfur, or phosphorus. The cycloalkenyl group and heterocycloalkenyl group can be substituted or unsubstituted. The cycloalkenyl group and heterocycloalkenyl group can be substituted with one or more groups including, but not limited to, alkyl, cycloalkyl, alkoxy, alkenyl, cycloalkenyl, alkynyl, cycloalkynyl, aryl, heteroaryl, aldehyde, amino, carboxylic acid, ester, ether, halide, hydroxy, ketone, azide, nitro, silyl, sulfo-oxo, or thiol as described herein.

The term “alkynyl” as used herein is a hydrocarbon group of 2 to 24 carbon atoms with a structural formula containing at least one carbon-carbon triple bond. The alkynyl group can be unsubstituted or substituted with one or more groups including, but not limited to, alkyl, cycloalkyl, alkoxy, alkenyl, cycloalkenyl, alkynyl, cycloalkynyl, aryl, heteroaryl, aldehyde, amino, carboxylic acid, ester, ether, halide, hydroxy, ketone, azide, nitro, silyl, sulfo-oxo, or thiol, as described herein.

The term “cycloalkynyl” as used herein is a non-aromatic carbon-based ring composed of at least seven carbon atoms and containing at least one carbon-carbon triple bound. Examples of cycloalkynyl groups include, but are not limited to, cycloheptynyl, cyclooctynyl, cyclononynyl, and the like. The term “heterocycloalkynyl” is a type of cycloalkenyl group as defined above, and is included within the meaning of the term “cycloalkynyl,” where at least one of the carbon atoms of the ring is replaced with a heteroatom such as, but not limited to, nitrogen, oxygen, sulfur, or phosphorus. The cycloalkynyl group and heterocycloalkynyl group can be substituted or unsubstituted. The cycloalkynyl group and heterocycloalkynyl group can be substituted with one or more groups including, but not limited to, alkyl, cycloalkyl, alkoxy, alkenyl, cycloalkenyl, alkynyl, cycloalkynyl, aryl, heteroaryl, aldehyde, amino, carboxylic acid, ester, ether, halide, hydroxy, ketone, azide, nitro, silyl, sulfo-oxo, or thiol as described herein.

The term “aromatic group” as used herein refers to a ring structure having cyclic clouds of delocalized π electrons above and below the plane of the molecule, where the π clouds contain (4n+2) π electrons. A further discussion of aromaticity is found in Morrison and Boyd, Organic Chemistry, (5th Ed., 1987), Chapter 13, entitled “Aromaticity,” pages 477-497, incorporated herein by reference. The term “aromatic group” is inclusive of both aryl and heteroaryl groups.

The term “aryl” as used herein is a group that contains any carbon-based aromatic group including, but not limited to, benzene, naphthalene, phenyl, biphenyl, anthracene, and the like. The aryl group can be substituted or unsubstituted. The aryl group can be substituted with one or more groups including, but not limited to, alkyl, cycloalkyl, alkoxy, alkenyl, cycloalkenyl, alkynyl, cycloalkynyl, aryl, heteroaryl, aldehyde, —NH₂, carboxylic acid, ester, ether, halide, hydroxy, ketone, azide, nitro, silyl, sulfo-oxo, or thiol as described herein. The term “biaryl” is a specific type of aryl group and is included in the definition of “aryl.” In addition, the aryl group can be a single ring structure or comprise multiple ring structures that are either fused ring structures or attached via one or more bridging groups such as a carbon-carbon bond. For example, biaryl to two aryl groups that are bound together via a fused ring structure, as in naphthalene, or are attached via one or more carbon-carbon bonds, as in biphenyl.

The term “aldehyde” as used herein is represented by the formula —C(O)H. Throughout this specification “C(O)” is a short hand notation for a carbonyl group, i.e., C═O.

The terms “amine” or “amino” as used herein are represented by the formula —NA¹A², where A¹ and A² can be, independently, hydrogen or alkyl, cycloalkyl, alkenyl, cycloalkenyl, alkynyl, cycloalkynyl, aryl, or heteroaryl group as described herein. A specific example of amino is —NH₂.

The term “alkylamino” as used herein is represented by the formula —NH(-alkyl) and —N(-alkyl)₂, where alkyl is a described herein. Representative examples include, but are not limited to, methylamino group, ethylamino group, propylamino group, isopropylamino group, butylamino group, isobutylamino group, (sec-butyl)amino group, (tert-butyl)amino group, pentylamino group, isopentylamino group, (tert-pentyl)amino group, hexylamino group, dimethylamino group, diethylamino group, dipropylamino group, diisopropylamino group, dibutylamino group, diisobutylamino group, di(sec-butyl)amino group, di(tert-butyl)amino group, dipentylamino group, diisopentylamino group, di(tert-pentyl)amino group, dihexylamino group, N-ethyl-N-methylamino group, N-methyl-N-propylamino group, N-ethyl-N-propylamino group and the like.

The term “carboxylic acid” as used herein is represented by the formula —C(O)OH.

The term “ester” as used herein is represented by the formula —OC(O)A¹ or —C(O)OA¹, where A¹ can be alkyl, cycloalkyl, alkenyl, cycloalkenyl, alkynyl, cycloalkynyl, aryl, or heteroaryl group as described herein. The term “polyester” as used herein is represented by the formula -(A¹O(O)C-A²-C(O)O)ₐ— or -(A¹O(O)C-A²-OC(O))ₐ—, where A¹ and A² can be, independently, an alkyl, cycloalkyl, alkenyl, cycloalkenyl, alkynyl, cycloalkynyl, aryl, or heteroaryl group described herein and “a” is an integer from 1 to 500. “Polyester” is as the term used to describe a group that is produced by the reaction between a compound having at least two carboxylic acid groups with a compound having at least two hydroxyl groups.

The term “ether” as used herein is represented by the formula A¹OA², where A¹ and A² can be, independently, an alkyl, cycloalkyl, alkenyl, cycloalkenyl, alkynyl, cycloalkynyl, aryl, or heteroaryl group described herein. The term “polyether” as used herein is represented by the formula -(A¹O-A²O)ₐ—, where A¹ and A² can be, independently, an alkyl, cycloalkyl, alkenyl, cycloalkenyl, alkynyl, cycloalkynyl, aryl, or heteroaryl group described herein and “a” is an integer of from 1 to 500. Examples of polyether groups include polyethylene oxide, polypropylene oxide, and polybutylene oxide.

The terms “halo,” “halogen” or “halide,” as used herein can be used interchangeably and refer to F, Cl, Br, or I.

The terms “pseudohalide,” “pseudohalogen” or “pseudohalo,” as used herein can be used interchangeably and refer to functional groups that behave substantially similar to halides. Such functional groups include, by way of example, cyano, thiocyanato, azido, trifluoromethyl, trifluoromethoxy, perfluoroalkyl, and perfluoroalkoxy groups.

The term “heteroalkyl” as used herein refers to an alkyl group containing at least one heteroatom. Suitable heteroatoms include, but are not limited to, O, N, Si, P and S, wherein the nitrogen, phosphorous and sulfur atoms are optionally oxidized, and the nitrogen heteroatom is optionally quaternized. Heteroalkyls can be substituted as defined above for alkyl groups.

The term “heteroaryl” as used herein refers to an aromatic group that has at least one heteroatom incorporated within the ring of the aromatic group. Examples of heteroatoms include, but are not limited to, nitrogen, oxygen, sulfur, and phosphorus, where N-oxides, sulfur oxides, and dioxides are permissible heteroatom substitutions. The heteroaryl group can be substituted or unsubstituted. The heteroaryl group can be substituted with one or more groups including, but not limited to, alkyl, cycloalkyl, alkoxy, amino, ether, halide, hydroxy, nitro, silyl, sulfo-oxo, or thiol as described herein. Heteroaryl groups can be monocyclic, or alternatively fused ring systems. Heteroaryl groups include, but are not limited to, furyl, imidazolyl, pyrimidinyl, tetrazolyl, thienyl, pyridinyl, pyrrolyl, N-methylpyrrolyl, quinolinyl, isoquinolinyl, pyrazolyl, triazolyl, thiazolyl, oxazolyl, isoxazolyl, oxadiazolyl, thiadiazolyl, isothiazolyl, pyridazinyl, pyrazinyl, benzofuranyl, benzodioxolyl, benzothiophenyl, indolyl, indazolyl, benzimidazolyl, imidazopyridinyl, pyrazolopyridinyl, and pyrazolopyrimidinyl. Further not limiting examples of heteroaryl groups include, but are not limited to, pyridinyl, pyridazinyl, pyrimidinyl, pyrazinyl, thiophenyl, pyrazolyl, imidazolyl, benzo[d]oxazolyl, benzo[d]thiazolyl, quinolinyl, quinazolinyl, indazolyl, imidazo[1,2-b]pyridazinyl, imidazo[1,2-a]pyrazinyl, benzo[c][1,2,5]thiadiazolyl, benzo[c][1,2,5]oxadiazolyl, and pyrido[2,3-b]pyrazinyl.

The terms “heterocycle” or “heterocyclyl,” as used herein can be used interchangeably and refer to single and multi-cyclic aromatic or non-aromatic ring systems in which at least one of the ring members is other than carbon. Thus, the term is inclusive of, but not limited to, “heterocycloalkyl,” “heteroaryl,” “bicyclic heterocycle,” and “polycyclic heterocycle.” Heterocycle includes pyridine, pyrimidine, furan, thiophene, pyrrole, isoxazole, isothiazole, pyrazole, oxazole, thiazole, imidazole, oxazole, including, 1,2,3-oxadiazole, 1,2,5-oxadiazole and 1,3,4-oxadiazole, thiadiazole, including, 1,2,3-thiadiazole, 1,2,5-thiadiazole, and 1,3,4-thiadiazole, triazole, including, 1,2,3-triazole, 1,3,4-triazole, tetrazole, including 1,2,3,4-tetrazole and 1,2,4,5-tetrazole, pyridazine, pyrazine, triazine, including 1,2,4-triazine and 1,3,5-triazine, tetrazine, including 1,2,4,5-tetrazine, pyrrolidine, piperidine, piperazine, morpholine, azetidine, tetrahydropyran, tetrahydrofuran, dioxane, and the like. The term heterocyclyl group can also be a C2 heterocyclyl, C2-C3 heterocyclyl, C2-C4 heterocyclyl, C2-C5 heterocyclyl, C2-C6 heterocyclyl, C2-C7 heterocyclyl, C2-C8 heterocyclyl, C2-C9 heterocyclyl, C2-C10 heterocyclyl, C2-C11 heterocyclyl, and the like up to and including a C2-C18 heterocyclyl. For example, a C2 heterocyclyl comprises a group which has two carbon atoms and at least one heteroatom, including, but not limited to, aziridinyl, diazetidinyl, dihydrodiazetyl, oxiranyl, thiiranyl, and the like. Alternatively, for example, a C5 heterocyclyl comprises a group which has five carbon atoms and at least one heteroatom, including, but not limited to, piperidinyl, tetrahydropyranyl, tetrahydrothiopyranyl, diazepanyl, pyridinyl, and the like. It is understood that a heterocyclyl group may be bound either through a heteroatom in the ring, where chemically possible, or one of carbons comprising the heterocyclyl ring.

The term “bicyclic heterocycle” or “bicyclic heterocyclyl” as used herein refers to a ring system in which at least one of the ring members is other than carbon. Bicyclic heterocyclyl encompasses ring systems wherein an aromatic ring is fused with another aromatic ring, or wherein an aromatic ring is fused with a non-aromatic ring. Bicyclic heterocyclyl encompasses ring systems wherein a benzene ring is fused to a 5- or a 6-membered ring containing 1, 2 or 3 ring heteroatoms or wherein a pyridine ring is fused to a 5- or a 6-membered ring containing 1, 2 or 3 ring heteroatoms. Bicyclic heterocyclic groups include, but are not limited to, indolyl, indazolyl, pyrazolo[1,5-a]pyridinyl, benzofuranyl, quinolinyl, quinoxalinyl, 1,3-benzodioxolyl, 2,3-dihydro-1,4-benzodioxinyl, 3,4-dihydro-2H-chromenyl, 1H-pyrazolo[4,3-c]pyridin-3-yl; 1H-pyrrolo[3,2-b]pyridin-3-yl; and 1H-pyrazolo[3,2-b]pyridin-3-yl.

The term “heterocycloalkyl” as used herein refers to an aliphatic, partially unsaturated or fully saturated, 3- to 14-membered ring system, including single rings of 3 to 8 atoms and bi- and tricyclic ring systems. The heterocycloalkyl ring-systems include one to four heteroatoms independently selected from oxygen, nitrogen, and sulfur, wherein a nitrogen and sulfur heteroatom optionally can be oxidized and a nitrogen heteroatom optionally can be substituted.

Representative heterocycloalkyl groups include, but are not limited to, pyrrolidinyl, pyrazolinyl, pyrazolidinyl, imidazolinyl, imidazolidinyl, piperidinyl, piperazinyl, oxazolidinyl, isoxazolidinyl, morpholinyl, thiazolidinyl, isothiazolidinyl, and tetrahydrofuryl.

The term “hydroxyl” or “hydroxy” as used herein is represented by the formula —OH.

The term “ketone” as used herein is represented by the formula A¹C(O)A², where A¹ and A² can be, independently, an alkyl, cycloalkyl, alkenyl, cycloalkenyl, alkynyl, cycloalkynyl, aryl, or heteroaryl group as described herein.

The term “azide” or “azido” as used herein is represented by the formula —N₃.

The term “nitro” as used herein is represented by the formula —NO₂.

The term “nitrile” or “cyano” as used herein is represented by the formula —CN.

The term “silyl” as used herein is represented by the formula —SiA¹A²A³, where A¹, A², and A³ can be, independently, hydrogen or an alkyl, cycloalkyl, alkoxy, alkenyl, cycloalkenyl, alkynyl, cycloalkynyl, aryl, or heteroaryl group as described herein.

The term “sulfo-oxo” as used herein is represented by the formulas —S(O)A¹, —S(O)₂A¹, —OS(O)₂A¹, or —OS(O)₂OA¹, where A¹ can be hydrogen or an alkyl, cycloalkyl, alkenyl, cycloalkenyl, alkynyl, cycloalkynyl, aryl, or heteroaryl group as described herein. Throughout this specification “S(O)” is a short hand notation for S═O. The term “sulfonyl” is used herein to refer to the sulfo-oxo group represented by the formula —S(O)₂A¹, where A¹ can be hydrogen or an alkyl, cycloalkyl, alkenyl, cycloalkenyl, alkynyl, cycloalkynyl, aryl, or heteroaryl group as described herein. The term “sulfone” as used herein is represented by the formula A¹S(O)₂A², where A¹ and A² can be, independently, an alkyl, cycloalkyl, alkenyl, cycloalkenyl, alkynyl, cycloalkynyl, aryl, or heteroaryl group as described herein. The term “sulfoxide” as used herein is represented by the formula A¹S(O)A², where A¹ and A² can be, independently, an alkyl, cycloalkyl, alkenyl, cycloalkenyl, alkynyl, cycloalkynyl, aryl, or heteroaryl group as described herein.

The term “thiol” as used herein is represented by the formula —SH.

“R¹,” “R²,” “R³,” . . . “Rⁿ,” where n is an integer, as used herein can, independently, possess one or more of the groups listed above. For example, if R¹ is a straight chain alkyl group, one of the hydrogen atoms of the alkyl group can optionally be substituted with a hydroxyl group, an alkoxy group, an alkyl group, a halide, and the like. Depending upon the groups that are selected, a first group can be incorporated within second group or, alternatively, the first group can be pendant (i.e., attached) to the second group. For example, with the phrase “an alkyl group comprising an amino group,” the amino group can be incorporated within the backbone of the alkyl group. Alternatively, the amino group can be attached to the backbone of the alkyl group. The nature of the group(s) that is (are) selected will determine if the first group is embedded or attached to the second group.

As described herein, compounds of the invention may contain “optionally substituted” moieties. In general, the term “substituted,” whether preceded by the term “optionally” or not, means that one or more hydrogens of the designated moiety are replaced with a suitable substituent. Unless otherwise indicated, an “optionally substituted” group may have a suitable substituent at each substitutable position of the group, and when more than one position in any given structure may be substituted with more than one substituent selected from a specified group, the substituent may be either the same or different at every position. Combinations of substituents envisioned by this invention are preferably those that result in the formation of stable or chemically feasible compounds. In is also contemplated that, in certain aspects, unless expressly indicated to the contrary, individual substituents can be further optionally substituted (i.e., further substituted or unsubstituted).

The term “stable,” as used herein, refers to compounds that are not substantially altered when subjected to conditions to allow for their production, detection, and, in certain aspects, their recovery, purification, and use for one or more of the purposes disclosed herein.

Suitable monovalent substituents on a substitutable carbon atom of an “optionally substituted” group are independently halogen; —(CH₂)₀₋₄R^{∘}; —(CH₂)₀₋₄OR^{∘}; —O(CH₂)₀₋₄R^{∘}, —O—(CH₂)₀₋₄C(O)OR^{∘}; —(CH₂)₀₋₄CH(OR^{∘})₂; —(CH₂)₀₋₄SR^{∘}; —(CH₂)₀₋₄Ph, which may be substituted with R^{∘}; —(CH₂)₀₋₄O(CH₂)₀₋₁Ph which may be substituted with R^{∘}; —CH═CHPh, which may be substituted with R^{∘}; —(CH₂)₀₋₄O(CH₂)₀₋₁-pyridyl which may be substituted with R^{∘}; —NO₂; —CN; —N₃; —(CH₂)₀₋₄N(R^{∘})₂; —(CH₂)₀₋₄N(R^{∘})C(O)R^{∘}; —N(R^{∘})C(S)R^{∘}; —(CH₂)₀₋₄N(R^{∘})C(O)NR^{∘}₂; —N(R^{∘})C(S)NR^{∘}₂; —(CH₂)₀₋₄N(R^{∘})C(O)OR^{∘}; —N(R^{∘})N(R^{∘})C(O)R^{∘}; —N(R^{∘})N(R^{∘})C(O)NR^{∘}₂; —N(R^{∘})N(R^{∘})C(O)OR^{∘}; —(CH₂)₀₋₄C(O)R^{∘}; —C(S)R^{∘}; —(CH₂)₀₋₄C(O)OR^{∘}; —(CH₂)₀₋₄C(O)SR^{∘}; —(CH₂)₀₋₄C(O)OSiR^{∘}₃; —(CH₂)₀₋₄OC(O)R^{∘}; —OC(O)(CH₂)₀₋₄SR—, SC(S)SR^{∘}; —(CH₂)₀₋₄SC(O)R^{∘}; —(CH₂)₀₋₄C(O)NR^{∘}₂; —C(S)NR^{∘}₂; —C(S)SR^{∘}; —(CH₂)₀₋₄OC(O)NR^{∘}₂; —C(O)N(OR^{∘})R^{∘}; —C(O)C(O)R^{∘}; —C(O)CH₂C(O)R^{∘}; —C(NOR^{∘})R^{∘}; —(CH₂)₀₋₄SSR^{∘}; —(CH₂)₀₋₄S(O)₂R^{∘}; —(CH₂)₀₋₄S(O)₂OR^{∘}; —(CH₂)₀₋₄OS(O)₂R^{∘}; —S(O)₂NR^{∘}₂; —(CH₂)₀₋₄S(O)R^{∘}; —N(R^{∘})S(O)₂NR^{∘}₂; —N(R^{∘})S(O)₂R^{∘}; —N(OR^{∘})R^{∘}; —C(NH)NR^{∘}₂; —P(O)₂R^{∘}; —P(O)R^{∘}₂; —OP(O)R^{∘}₂; —OP(O)(OR^{∘})₂; SiR^{∘}₃; —(C₁₋₄ straight or branched alkylene)O—N(R^{∘})₂; or —(C₁₋₄ straight or branched alkylene)C(O)O—N(R^{∘})₂, wherein each R^{∘} may be substituted as defined below and is independently hydrogen, C₁₋₆ aliphatic, —CH₂Ph, —O(CH₂)₀₋₁Ph, —CH₂-(5-6 membered heteroaryl ring), or a 5-6-membered saturated, partially unsaturated, or aryl ring having 0-4 heteroatoms independently selected from nitrogen, oxygen, or sulfur, or, notwithstanding the definition above, two independent occurrences of R^{∘}, taken together with their intervening atom(s), form a 3-12-membered saturated, partially unsaturated, or aryl mono- or bicyclic ring having 0-4 heteroatoms independently selected from nitrogen, oxygen, or sulfur, which may be substituted as defined below.

Suitable monovalent substituents on R^{∘} (or the ring formed by taking two independent occurrences of R^{∘} together with their intervening atoms), are independently halogen, —(CH₂)₀₋₂R^{●}, -(haloR^{●}), —(CH₂)₀₋₂OH, —(CH₂)₀₋₂OR^{●}, —(CH₂)₀₋₂CH(OR^{●})₂; —O(haloR^{●}), —CN, —N₃, —(CH₂)₀₋₂C(O)R^{●}, —(CH₂)₀₋₂C(O)OH, —(CH₂)₀₋₂C(O)OR^{●}, —(CH₂)₀₋₂SR^{●}, —(CH₂)₀₋₂SH, —(CH₂)₀₋₂NH₂, —(CH₂)₀₋₂NHR^{●}, —(CH₂)₀₋₂NR^{●}₂, —NO₂, —SiR^{●}₃, —OSiR^{●}₃, —C(O)SR^{●}, —(C₁₋₄ straight or branched alkylene)C(O)OR^{●}, or —SSR^{●} wherein each R^{●} is unsubstituted or where preceded by “halo” is substituted only with one or more halogens, and is independently selected from C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur. Suitable divalent substituents on a saturated carbon atom of R^{●} include ═O and ═S.

Suitable divalent substituents on a saturated carbon atom of an “optionally substituted” group include the following: ═O, ═S, ═NNR*₂, ═NNHC(O)R*, ═NNHC(O)OR*, ═NNHS(O)₂R*, ═NR*, ═NOR*, —O(C(R*₂))₂₋₃O—, or —S(C(R*₂))₂₋₃S—, wherein each independent occurrence of R* is selected from hydrogen, C₁₋₆ aliphatic which may be substituted as defined below, or an unsubstituted 5-6-membered saturated, partially unsaturated, or aryl ring having 0-4 heteroatoms independently selected from nitrogen, oxygen, or sulfur. Suitable divalent substituents that are bound to vicinal substitutable carbons of an “optionally substituted” group include: —O(CR*₂)₂₋₃O—, wherein each independent occurrence of R* is selected from hydrogen, C₁₋₆ aliphatic which may be substituted as defined below, or an unsubstituted 5-6-membered saturated, partially unsaturated, or aryl ring having 0-4 heteroatoms independently selected from nitrogen, oxygen, or sulfur.

Suitable substituents on the aliphatic group of R* include halogen, —R^{●}, -(haloR^{●}), —OH, —OR^{●}, —O(haloR^{●}), —CN, —C(O)OH, —C(O)OR^{●}, —NH₂, —NHR^{●}, —NR^{●}₂, or —NO₂, wherein each R^{●} is unsubstituted or where preceded by “halo” is substituted only with one or more halogens, and is independently C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur.

Suitable substituents on a substitutable nitrogen of an “optionally substituted” group include —R^{†}, —NR^{†}₂, —C(O)R^{†}, —C(O)OR^{†}, —C(O)C(O)R^{†}, —C(O)CH₂C(O)R^{†}, —S(O)₂R^{†}, —S(O)₂NR^{†}₂, —C(S)NR^{†}₂, —C(NH)NR^{†}₂, or —N(R^{†})S(O)₂R^{†}; wherein each R^{†} is independently hydrogen, C₁₋₆ aliphatic which may be substituted as defined below, unsubstituted —OPh, or an unsubstituted 5-6-membered saturated, partially unsaturated, or aryl ring having 0-4 heteroatoms independently selected from nitrogen, oxygen, or sulfur, or, notwithstanding the definition above, two independent occurrences of R^{†}, taken together with their intervening atom(s) form an unsubstituted 3-12-membered saturated, partially unsaturated, or aryl mono- or bicyclic ring having 0-4 heteroatoms independently selected from nitrogen, oxygen, or sulfur.

Suitable substituents on the aliphatic group of R^{†} are independently halogen, —R^{●}, -(haloR^{●}), —OH, —OR^{●}, —O(haloR^{●}), —CN, —C(O)OH, —C(O)OR^{●}, —NH₂, —NHR^{●}, —NR^{●}₂, or —NO₂, wherein each R^{●} is unsubstituted or where preceded by “halo” is substituted only with one or more halogens, and is independently C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur.

The term “leaving group” refers to an atom (or a group of atoms) with electron withdrawing ability that can be displaced as a stable species, taking with it the bonding electrons. Examples of suitable leaving groups include halides and sulfonate esters, including, but not limited to, triflate, mesylate, tosylate, and brosylate.

The terms “hydrolysable group” and “hydrolysable moiety” refer to a functional group capable of undergoing hydrolysis, e.g., under basic or acidic conditions. Examples of hydrolysable residues include, without limitation, acid halides, activated carboxylic acids, and various protecting groups known in the art (see, for example, “Protective Groups in Organic Synthesis,” T. W. Greene, P. G. M. Wuts, Wiley-Interscience, 1999).

The term “organic residue” defines a carbon containing residue, i.e., a residue comprising at least one carbon atom, and includes but is not limited to the carbon-containing groups, residues, or radicals defined hereinabove. Organic residues can contain various heteroatoms, or be bonded to another molecule through a heteroatom, including oxygen, nitrogen, sulfur, phosphorus, or the like. Examples of organic residues include but are not limited alkyl or substituted alkyls, alkoxy or substituted alkoxy, mono or di-substituted amino, amide groups, etc. Organic residues can preferably comprise 1 to 18 carbon atoms, 1 to 15, carbon atoms, 1 to 12 carbon atoms, 1 to 8 carbon atoms, 1 to 6 carbon atoms, or 1 to 4 carbon atoms. In a further aspect, an organic residue can comprise 2 to 18 carbon atoms, 2 to 15, carbon atoms, 2 to 12 carbon atoms, 2 to 8 carbon atoms, 2 to 4 carbon atoms, or 2 to 4 carbon atoms.

A very close synonym of the term “residue” is the term “radical,” which as used in the specification and concluding claims, refers to a fragment, group, or substructure of a molecule described herein, regardless of how the molecule is prepared. For example, a 2,4-thiazolidinedione radical in a particular compound has the structure:

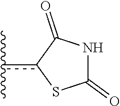

regardless of whether thiazolidinedione is used to prepare the compound. In some embodiments the radical (for example an alkyl) can be further modified (i.e., substituted alkyl) by having bonded thereto one or more “substituent radicals.” The number of atoms in a given radical is not critical to the present invention unless it is indicated to the contrary elsewhere herein.

“Organic radicals,” as the term is defined and used herein, contain one or more carbon atoms. An organic radical can have, for example, 1-26 carbon atoms, 1-18 carbon atoms, 1-12 carbon atoms, 1-8 carbon atoms, 1-6 carbon atoms, or 1-4 carbon atoms. In a further aspect, an organic radical can have 2-26 carbon atoms, 2-18 carbon atoms, 2-12 carbon atoms, 2-8 carbon atoms, 2-6 carbon atoms, or 2-4 carbon atoms. Organic radicals often have hydrogen bound to at least some of the carbon atoms of the organic radical. One example of an organic radical that comprises no inorganic atoms is a 5, 6, 7, 8-tetrahydro-2-naphthyl radical. In some embodiments, an organic radical can contain 1-10 inorganic heteroatoms bound thereto or therein, including halogens, oxygen, sulfur, nitrogen, phosphorus, and the like. Examples of organic radicals include but are not limited to an alkyl, substituted alkyl, cycloalkyl, substituted cycloalkyl, mono-substituted amino, di-substituted amino, acyloxy, cyano, carboxy, carboalkoxy, alkylcarboxamide, substituted alkylcarboxamide, dialkylcarboxamide, substituted dialkylcarboxamide, alkylsulfonyl, alkylsulfinyl, thioalkyl, thiohaloalkyl, alkoxy, substituted alkoxy, haloalkyl, haloalkoxy, aryl, substituted aryl, heteroaryl, heterocyclic, or substituted heterocyclic radicals, wherein the terms are defined elsewhere herein. A few non-limiting examples of organic radicals that include heteroatoms include alkoxy radicals, trifluoromethoxy radicals, acetoxy radicals, dimethylamino radicals and the like.

“Inorganic radicals,” as the term is defined and used herein, contain no carbon atoms and therefore comprise only atoms other than carbon. Inorganic radicals comprise bonded combinations of atoms selected from hydrogen, nitrogen, oxygen, silicon, phosphorus, sulfur, selenium, and halogens such as fluorine, chlorine, bromine, and iodine, which can be present individually or bonded together in their chemically stable combinations. Inorganic radicals have 10 or fewer, or preferably one to six or one to four inorganic atoms as listed above bonded together. Examples of inorganic radicals include, but not limited to, amino, hydroxy, halogens, nitro, thiol, sulfate, phosphate, and like commonly known inorganic radicals. The inorganic radicals do not have bonded therein the metallic elements of the periodic table (such as the alkali metals, alkaline earth metals, transition metals, lanthanide metals, or actinide metals), although such metal ions can sometimes serve as a pharmaceutically acceptable cation for anionic inorganic radicals such as a sulfate, phosphate, or like anionic inorganic radical. Inorganic radicals do not comprise metalloids elements such as boron, aluminum, gallium, germanium, arsenic, tin, lead, or tellurium, or the noble gas elements, unless otherwise specifically indicated elsewhere herein.

Compounds described herein can contain one or more double bonds and, thus, potentially give rise to cis/trans (E/Z) isomers, as well as other conformational isomers. Unless stated to the contrary, the invention includes all such possible isomers, as well as mixtures of such isomers.

Unless stated to the contrary, a formula with chemical bonds shown only as solid lines and not as wedges or dashed lines contemplates each possible isomer, e.g., each enantiomer and diastereomer, and a mixture of isomers, such as a racemic or scalemic mixture. Compounds described herein can contain one or more asymmetric centers and, thus, potentially give rise to diastereomers and optical isomers. Unless stated to the contrary, the present invention includes all such possible diastereomers as well as their racemic mixtures, their substantially pure resolved enantiomers, all possible geometric isomers, and pharmaceutically acceptable salts thereof.

Mixtures of stereoisomers, as well as isolated specific stereoisomers, are also included. During the course of the synthetic procedures used to prepare such compounds, or in using racemization or epimerization procedures known to those skilled in the art, the products of such procedures can be a mixture of stereoisomers.

Many organic compounds exist in optically active forms having the ability to rotate the plane of plane-polarized light. In describing an optically active compound, the prefixes D and L or R and S are used to denote the absolute configuration of the molecule about its chiral center(s). The prefixes d and l or (+) and (−) are employed to designate the sign of rotation of plane-polarized light by the compound, with (−) or meaning that the compound is levorotatory. A compound prefixed with (+) or d is dextrorotatory. For a given chemical structure, these compounds, called stereoisomers, are identical except that they are non-superimposable mirror images of one another. A specific stereoisomer can also be referred to as an enantiomer, and a mixture of such isomers is often called an enantiomeric mixture. A 50:50 mixture of enantiomers is referred to as a racemic mixture. Many of the compounds described herein can have one or more chiral centers and therefore can exist in different enantiomeric forms. If desired, a chiral carbon can be designated with an asterisk (*). When bonds to the chiral carbon are depicted as straight lines in the disclosed formulas, it is understood that both the (R) and (S) configurations of the chiral carbon, and hence both enantiomers and mixtures thereof, are embraced within the formula. As is used in the art, when it is desired to specify the absolute configuration about a chiral carbon, one of the bonds to the chiral carbon can be depicted as a wedge (bonds to atoms above the plane) and the other can be depicted as a series or wedge of short parallel lines is (bonds to atoms below the plane). The Cahn-Ingold-Prelog system can be used to assign the (R) or (S) configuration to a chiral carbon.

Compounds described herein comprise atoms in both their natural isotopic abundance and in non-natural abundance. The disclosed compounds can be isotopically-labeled or isotopically-substituted compounds identical to those described, but for the fact that one or more atoms are replaced by an atom having an atomic mass or mass number different from the atomic mass or mass number typically found in nature. Examples of isotopes that can be incorporated into compounds of the invention include isotopes of hydrogen, carbon, nitrogen, oxygen, sulfur, fluorine, and chlorine, such as ²H, ³H, ¹³C, ¹⁴C, ¹⁵N, ¹⁸O, ¹⁷O, ³⁵S, ¹⁸F, and ³⁶Cl, respectively. Compounds further comprise prodrugs thereof and pharmaceutically acceptable salts of said compounds or of said prodrugs which contain the aforementioned isotopes and/or other isotopes of other atoms are within the scope of this invention. Certain isotopically-labeled compounds of the present invention, for example those into which radioactive isotopes such as ³H and ¹⁴C are incorporated, are useful in drug and/or substrate tissue distribution assays. Tritiated, i.e., ³H, and carbon-14, i.e., ¹⁴C, isotopes are particularly preferred for their ease of preparation and detectability. Further, substitution with heavier isotopes such as deuterium, i.e., ²H, can afford certain therapeutic advantages resulting from greater metabolic stability, for example increased in vivo half-life or reduced dosage requirements and, hence, may be preferred in some circumstances. Isotopically labeled compounds of the present invention and prodrugs thereof can generally be prepared by carrying out the procedures below, by substituting a readily available isotopically labeled reagent for a non-isotopically labeled reagent.

The compounds described in the invention can be present as a solvate. In some cases, the solvent used to prepare the solvate is an aqueous solution, and the solvate is then often referred to as a hydrate. The compounds can be present as a hydrate, which can be obtained, for example, by crystallization from a solvent or from aqueous solution. In this connection, one, two, three or any arbitrary number of solvent or water molecules can combine with the compounds according to the invention to form solvates and hydrates. Unless stated to the contrary, the invention includes all such possible solvates.

It is also appreciated that certain compounds described herein can be present as an equilibrium of tautomers. For example, ketones with an α-hydrogen can exist in an equilibrium of the keto form and the enol form.

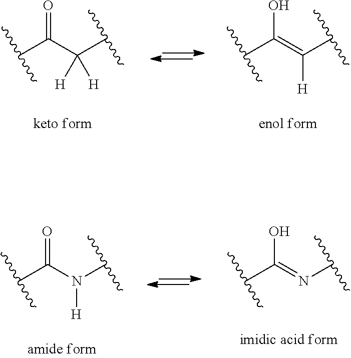 [SMILES: *CC(*)=O ; */C=C(/*)O ; *NC(*)=O ; */N=C(/*)O]

Likewise, amides with an N-hydrogen can exist in an equilibrium of the amide form and the imidic acid form. Unless stated to the contrary, the invention includes all such possible tautomers.

It is known that chemical substances form solids which are present in different states of order which are termed polymorphic forms or modifications. The different modifications of a polymorphic substance can differ greatly in their physical properties. The compounds according to the invention can be present in different polymorphic forms, with it being possible for particular modifications to be metastable. Unless stated to the contrary, the invention includes all such possible polymorphic forms.

In some aspects, a structure of a compound can be represented by a formula:

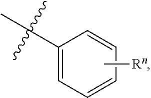

which is understood to be equivalent to a formula:

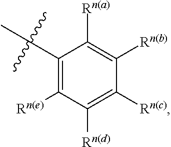

wherein n is typically an integer. That is, Rⁿ is understood to represent five independent substituents, R^{n(a)}, R_{n(b)}, R^{n(c)}, R^{n(d)}, and R^{n(e)}. By “independent substituents,” it is meant that each R substituent can be independently defined. For example, if in one instance Rₙ₍ₐ₎ is halogen, then R^{n(b)} is not necessarily halogen in that instance.

Certain materials, compounds, compositions, and components disclosed herein can be obtained commercially or readily synthesized using techniques generally known to those of skill in the art. For example, the starting materials and reagents used in preparing the disclosed compounds and compositions are either available from commercial suppliers such as Aldrich Chemical Co., (Milwaukee, Wis.), Acros Organics (Morris Plains, N.J.), Fisher Scientific (Pittsburgh, Pa.), or Sigma (St. Louis, Mo.) or are prepared by methods known to those skilled in the art following procedures set forth in references such as Fieser and Fieser's Reagents for Organic Synthesis, Volumes 1-17 (John Wiley and Sons, 1991); Rodd's Chemistry of Carbon Compounds, Volumes 1-5 and Supplementals (Elsevier Science Publishers, 1989); Organic Reactions, Volumes 1-40 (John Wiley and Sons, 1991); March's Advanced Organic Chemistry, (John Wiley and Sons, 4th Edition); and Larock's Comprehensive Organic Transformations (VCH Publishers Inc., 1989).

Unless otherwise expressly stated, it is in no way intended that any method set forth herein be construed as requiring that its steps be performed in a specific order. Accordingly, where a method claim does not actually recite an order to be followed by its steps or it is not otherwise specifically stated in the claims or descriptions that the steps are to be limited to a specific order, it is no way intended that an order be inferred, in any respect. This holds for any possible non-express basis for interpretation, including: matters of logic with respect to arrangement of steps or operational flow; plain meaning derived from grammatical organization or punctuation; and the number or type of embodiments described in the specification.

Disclosed are the components to be used to prepare the compositions of the invention as well as the compositions themselves to be used within the methods disclosed herein. These and other materials are disclosed herein, and it is understood that when combinations, subsets, interactions, groups, etc. of these materials are disclosed that while specific reference of each various individual and collective combinations and permutation of these compounds cannot be explicitly disclosed, each is specifically contemplated and described herein. For example, if a particular compound is disclosed and discussed and a number of modifications that can be made to a number of molecules including the compounds are discussed, specifically contemplated is each and every combination and permutation of the compound and the modifications that are possible unless specifically indicated to the contrary. Thus, if a class of molecules A, B, and C are disclosed as well as a class of molecules D, E, and F and an example of a combination molecule, A-D is disclosed, then even if each is not individually recited each is individually and collectively contemplated meaning combinations, A-E, A-F, B-D, B-E, B-F, C-D, C-E, and C-F are considered disclosed. Likewise, any subset or combination of these is also disclosed. Thus, for example, the sub-group of A-E, B-F, and C-E would be considered disclosed. This concept applies to all aspects of this application including, but not limited to, steps in methods of making and using the compositions of the invention. Thus, if there are a variety of additional steps that can be performed it is understood that each of these additional steps can be performed with any specific embodiment or combination of embodiments of the methods of the invention.

It is understood that the compositions disclosed herein have certain functions. Disclosed herein are certain structural requirements for performing the disclosed functions, and it is understood that there are a variety of structures that can perform the same function that are related to the disclosed structures, and that these structures will typically achieve the same result.

In various aspects, it is contemplated herein that the disclosed compounds further comprise their biosteric equivalents. The term “bioisosteric equivalent” refers to compounds or groups that possess near equal molecular shapes and volumes, approximately the same distribution of electrons, and which exhibit similar physical and biological properties. Examples of such equivalents are: (i) fluorine vs. hydrogen, (ii) oxo vs. thia, (iii) hydroxyl vs. amide, (iv) carbonyl vs. oxime, (v) carboxylate vs. tetrazole. Examples of such bioisosteric replacements can be found in the literature and examples of such are: (i) Burger A, Relation of chemical structure and biological activity; in Medicinal Chemistry Third ed., Burger A, ed.; Wiley-Interscience; New York, 1970, 64-80; (ii) Burger, A.; “Isosterism and bioisosterism in drug design”; Prog. Drug Res. 1991, 37, 287-371; (iii) Burger A, “Isosterism and bioanalogy in drug design”, Med. Chem. Res. 1994, 4, 89-92; (iv) Clark R D, Ferguson A M, Cramer R D, “Bioisosterism and molecular diversity”, Perspect. Drug Discovery Des. 1998, 9/10/11, 213-224; (v) Koyanagi T, Haga T, “Bioisosterism in agrochemicals”, ACS Symp. Ser. 1995, 584, 15-24; (vi) Kubinyi H, “Molecular similarities. Part 1. Chemical structure and biological activity,” Pharm. Unserer Zeit 1998, 27, 92-106; (vii) Lipinski C A.; “Bioisosterism in drug design”; Annu. Rep. Med. Chem. 1986, 21, 283-91; (viii) Patani G A, LaVoie E J, “Bioisosterism: A rational approach in drug design”, Chem. Rev. (Washington, D.C.) 1996, 96, 3147-3176; (ix) Soskic V, Joksimovic J, “Bioisosteric approach in the design of new dopaminergic/serotonergic ligands”, Curr. Med. Chem. 1998, 5, 493-512 (x) Thornber C W, “Isosterism and molecular modification in drug design”, Chem. Soc. Rev. 1979, 8, 563-80.

In further aspects, bioisosteres are atoms, ions, or molecules in which the peripheral layers of electrons can be considered substantially identical. The term bioisostere is usually used to mean a portion of an overall molecule, as opposed to the entire molecule itself. Bioisosteric replacement involves using one bioisostere to replace another with the expectation of maintaining or slightly modifying the biological activity of the first bioisostere. The bioisosteres in this case are thus atoms or groups of atoms having similar size, shape, and electron density. Preferred bioisosteres of esters, amides or carboxylic acids are compounds containing two sites for hydrogen bond acceptance. In one embodiment, the ester, amide, or carboxylic acid bioisostere is a 5-membered monocyclic heteroaryl ring, such as an optionally substituted 1H-imidazolyl, an optionally substituted oxazolyl, 1H-tetrazolyl, [1,2,4]triazolyl, or an optionally substituted [1,2,4]oxadiazolyl.

As used herein, “administering” can refer to an administration that is oral, topical, intravenous, subcutaneous, transcutaneous, transdermal, intramuscular, intra-joint, parenteral, intra-arteriole, intradermal, intraventricular, intraosseous, intraocular, intracranial, intraperitoneal, intralesional, intranasal, intracardiac, intraarticular, intracavernous, intrathecal, intravireal, intracerebral, and intracerebroventricular, intratympanic, intracochlear, rectal, vaginal, by inhalation, by catheters, stents or via an implanted reservoir or other device that administers, either actively or passively (e.g. by diffusion) a composition the perivascular space and adventitia. For example, a medical device such as a stent can contain a composition or formulation disposed on its surface, which can then dissolve or be otherwise distributed to the surrounding tissue and cells. The term “parenteral” can include subcutaneous, intravenous, intramuscular, intra-articular, intra-synovial, intrasternal, intrathecal, intrahepatic, intralesional, and intracranial injections or infusion techniques. Administration can be continuous or intermittent. In various aspects, a preparation can be administered therapeutically; that is, administered to treat an existing disease or condition. In further various aspects, a preparation can be administered prophylactically; that is, administered for prevention of a disease or condition.

As used herein, “therapeutic agent” can refer to any substance, compound, molecule, and the like, which can be biologically active or otherwise can induce a pharmacologic, immunogenic, biologic and/or physiologic effect on a subject to which it is administered to by local and/or systemic action. A therapeutic agent can be a primary active agent, or in other words, the component(s) of a composition to which the whole or part of the effect of the composition is attributed. A therapeutic agent can be a secondary therapeutic agent, or in other words, the component(s) of a composition to which an additional part and/or other effect of the composition is attributed. The term therefore encompasses those compounds or chemicals traditionally regarded as drugs, vaccines, and biopharmaceuticals including molecules such as proteins, peptides, hormones, nucleic acids, gene constructs and the like. Examples of therapeutic agents are described in well-known literature references such as the Merck Index (14th edition), the Physicians' Desk Reference (64th edition), and The Pharmacological Basis of Therapeutics (12th edition), and they include, without limitation, medicaments; vitamins; mineral supplements; substances used for the treatment, prevention, diagnosis, cure or mitigation of a disease or illness; substances that affect the structure or function of the body, or pro-drugs, which become biologically active or more active after they have been placed in a physiological environment. For example, the term “therapeutic agent” includes compounds or compositions for use in all of the major therapeutic areas including, but not limited to, adjuvants; anti-infectives such as antibiotics and antiviral agents; analgesics and analgesic combinations, anorexics, anti-inflammatory agents, anti-epileptics, local and general anesthetics, hypnotics, sedatives, antipsychotic agents, neuroleptic agents, antidepressants, anxiolytics, antagonists, neuron blocking agents, anticholinergic and cholinomimetic agents, antimuscarinic and muscarinic agents, antiadrenergics, antiarrhythmics, antihypertensive agents, hormones, and nutrients, antiarthritics, antiasthmatic agents, anticonvulsants, antihistamines, antinauseants, antineoplastics, antipruritics, antipyretics; antispasmodics, cardiovascular preparations (including calcium channel blockers, beta-blockers, beta-agonists and antiarrythmics), antihypertensives, diuretics, vasodilators; central nervous system stimulants; cough and cold preparations; decongestants; diagnostics; hormones; bone growth stimulants and bone resorption inhibitors; immunosuppressives; muscle relaxants; psychostimulants; sedatives; tranquilizers; proteins, peptides, and fragments thereof (whether naturally occurring, chemically synthesized or recombinantly produced); and nucleic acid molecules (polymeric forms of two or more nucleotides, either ribonucleotides (RNA) or deoxyribonucleotides (DNA) including both double- and single-stranded molecules, gene constructs, expression vectors, antisense molecules and the like), small molecules (e.g., doxorubicin) and other biologically active macromolecules such as, for example, proteins and enzymes. The agent may be a biologically active agent used in medical, including veterinary, applications and in agriculture, such as with plants, as well as other areas. The term therapeutic agent also includes without limitation, medicaments; vitamins; mineral supplements; substances used for the treatment, prevention, diagnosis, cure or mitigation of disease or illness; or substances which affect the structure or function of the body; or pro-drugs, which become biologically active or more active after they have been placed in a predetermined physiological environment.

As used herein, an “antibiotic” or an “antibacterial agent” is a type of therapeutic agent targeting bacteria. In some aspects, the bacteria can be pathogenic bacteria that cause a human disease such as, for example, Mycobacterium tuberculosis (Mtb). In one aspect, certain strains of bacteria can be resistant to one or more antibiotics. Disclosed herein are antibiotic agents useful against both drug-resistant and drug-susceptible strains of Mtb.

As used herein, “attached” can refer to covalent or non-covalent interaction between two or more molecules. Non-covalent interactions can include ionic bonds, electrostatic interactions, van der Walls forces, dipole-dipole interactions, dipole-induced-dipole interactions, London dispersion forces, hydrogen bonding, halogen bonding, electromagnetic interactions, π-π interactions, cation-π interactions, anion-π interactions, polar π-interactions, and hydrophobic effects.

As used interchangeably herein, “subject,” “individual,” or “patient” can refer to a vertebrate organism, such as a mammal (e.g. human). “Subject” can also refer to a cell, a population of cells, a tissue, an organ, or an organism, preferably to human and constituents thereof.

As used herein, the terms “treating” and “treatment” can refer generally to obtaining a desired pharmacological and/or physiological effect. The effect can be, but does not necessarily have to be, prophylactic in terms of preventing or partially preventing a disease, symptom, or condition thereof, such as tuberculosis. The effect can be therapeutic in terms of a partial or complete cure of a disease, condition, symptom, or adverse effect attributed to the disease, disorder, or condition. The term “treatment” as used herein can include any treatment of tuberculosis in a subject, particularly a human and can include any one or more of the following: (a) preventing the disease from occurring in a subject who may have been exposed to the disease but has not yet been diagnosed as having it; (b) inhibiting the disease, i.e., arresting its development; and (c) relieving the disease, i.e., mitigating or ameliorating the disease and/or its symptoms or conditions. The term “treatment” as used herein can refer to both therapeutic treatment alone, prophylactic treatment alone, or both therapeutic and prophylactic treatment. Those in need of treatment (subjects in need thereof) can include those already with the disorder and/or those in which the disorder is to be prevented. As used herein, the term “treating”, can include inhibiting the disease, disorder, or condition, e.g., impeding its progress; and relieving the disease, disorder, or condition, e.g., causing regression of the disease, disorder and/or condition. Treating the disease, disorder, or condition can include ameliorating at least one symptom of the particular disease, disorder, or condition, even if the underlying pathophysiology is not affected, e.g., such as treating the pain of a subject by administration of an analgesic agent even though such agent does not treat the cause of the pain.

As used herein, “dose,” “unit dose,” or “dosage” can refer to physically discrete units suitable for use in a subject, each unit containing a predetermined quantity of a disclosed compound and/or a pharmaceutical composition thereof calculated to produce the desired response or responses in association with its administration.

As used herein, “therapeutic” can refer to treating, healing, and/or ameliorating a disease, disorder, condition, or side effect, or to decreasing in the rate of advancement of a disease, disorder, condition, or side effect.

As used herein, “effective amount” can refer to the amount of a disclosed compound or pharmaceutical composition provided herein that is sufficient to effect beneficial or desired biological, emotional, medical, or clinical response of a cell, tissue, system, animal, or human. An effective amount can be administered in one or more administrations, applications, or dosages. The term can also include within its scope amounts effective to enhance or restore to substantially normal physiological function.

As used herein, the term “therapeutically effective amount” refers to an amount that is sufficient to achieve the desired therapeutic result or to have an effect on undesired symptoms, but is generally insufficient to cause adverse side effects. The specific therapeutically effective dose level for any particular patient will depend upon a variety of factors including the disorder being treated and the severity of the disorder; the specific composition employed; the age, body weight, general health, sex and diet of the patient; the time of administration; the route of administration; the rate of excretion of the specific compound employed; the duration of the treatment; drugs used in combination or coincidental with the specific compound employed and like factors within the knowledge and expertise of the health practitioner and which may be well known in the medical arts. In the case of treating a particular disease or condition, in some instances, the desired response can be inhibiting the progression of the disease or condition. This may involve only slowing the progression of the disease temporarily. However, in other instances, it may be desirable to halt the progression of the disease permanently. This can be monitored by routine diagnostic methods known to one of ordinary skill in the art for any particular disease. The desired response to treatment of the disease or condition also can be delaying the onset or even preventing the onset of the disease or condition.

For example, it is well within the skill of the art to start doses of a compound at levels lower than those required to achieve the desired therapeutic effect and to gradually increase the dosage until the desired effect is achieved. If desired, the effective daily dose can be divided into multiple doses for purposes of administration. Consequently, single dose compositions can contain such amounts or submultiples thereof to make up the daily dose. The dosage can be adjusted by the individual physician in the event of any contraindications. It is generally preferred that a maximum dose of the pharmacological agents of the invention (alone or in combination with other therapeutic agents) be used, that is, the highest safe dose according to sound medical judgment. It will be understood by those of ordinary skill in the art however, that a patient may insist upon a lower dose or tolerable dose for medical reasons, psychological reasons or for virtually any other reasons.

A response to a therapeutically effective dose of a disclosed compound and/or pharmaceutical composition, for example, can be measured by determining the physiological effects of the treatment or medication, such as the decrease or lack of disease symptoms following administration of the treatment or pharmacological agent. Other assays will be known to one of ordinary skill in the art and can be employed for measuring the level of the response. The amount of a treatment may be varied for example by increasing or decreasing the amount of a disclosed compound and/or pharmaceutical composition, by changing the disclosed compound and/or pharmaceutical composition administered, by changing the route of administration, by changing the dosage timing and so on. Dosage can vary, and can be administered in one or more dose administrations daily, for one or several days. Guidance can be found in the literature for appropriate dosages for given classes of pharmaceutical products.

As used herein, the term “prophylactically effective amount” refers to an amount effective for preventing onset or initiation of a disease or condition.

As used herein, the term “prevent” or “preventing” refers to precluding, averting, obviating, forestalling, stopping, or hindering something from happening, especially by advance action. It is understood that where reduce, inhibit, or prevent are used herein, unless specifically indicated otherwise, the use of the other two words is also expressly disclosed.

The term “pharmaceutically acceptable” describes a material that is not biologically or otherwise undesirable, i.e., without causing an unacceptable level of undesirable biological effects or interacting in a deleterious manner.

The term “pharmaceutically acceptable salts,” as used herein, means salts of the active principal agents which are prepared with acids or bases that are tolerated by a biological system or tolerated by a subject or tolerated by a biological system and tolerated by a subject when administered in a therapeutically effective amount. When compounds of the present disclosure contain relatively acidic functionalities, base addition salts can be obtained by contacting the neutral form of such compounds with a sufficient amount of the desired base, either neat or in a suitable inert solvent. Examples of pharmaceutically acceptable base addition salts include, but are not limited to; sodium, potassium, calcium, ammonium, organic amino, magnesium salt, lithium salt, strontium salt or a similar salt. When compounds of the present disclosure contain relatively basic functionalities, acid addition salts can be obtained by contacting the neutral form of such compounds with a sufficient amount of the desired acid, either neat or in a suitable inert solvent. Examples of pharmaceutically acceptable acid addition salts include, but are not limited to; those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydriodic, or phosphorous acids and the like, as well as the salts derived from relatively nontoxic organic acids like acetic, propionic, isobutyric, maleic, malonic, benzoic, succinic, suberic, fumaric, lactic, mandelic, phthalic, benzenesulfonic, p-tolylsulfonic, citric, tartaric, methanesulfonic, and the like. Also included are salts of amino acids such as arginate and the like, and salts of organic acids like glucuronic or galactunoric acids and the like.

In one aspect, the disclosed compound can be present as a cation, for example, a charged tertiary amine under physiological conditions, or as a quaternary amine. Further in this aspect, the disclosed compound can be a salt having an anionic counter ion. In one aspect, the counter ion can be acetate, aspartate, benzenesulfonate, benzoate, besylate, bicarbonate, bitartrate, bromide, camsylate, carbonate, chloride, citrate, decanoate, edetate, esylate, fumarate, gluceptate, gluconate, glutamate, glycolate, hexanoate, hydroxynaphthoate, iodide, isethionate, lactate, lactobionate, malate, maleate, mandelate, mesylate, methylsulfate, mucate, napsylate, nitrate, octanoate, oleate, pamoate, pantothenate, phosphate, polygalacturonate, propionate, salicylate, stearate, succinate, sulfate, tartrate, teoclate, tosylate, or any combination thereof.

The term “pharmaceutically acceptable ester” refers to esters of compounds of the present disclosure which hydrolyze in vivo and include those that break down readily in the human body to leave the parent compound or a salt thereof. Examples of pharmaceutically acceptable, non-toxic esters of the present disclosure include C 1-to-C 6 alkyl esters and C 5-to-C 7 cycloalkyl esters, although C 1-to-C 4 alkyl esters are preferred. Esters of disclosed compounds can be prepared according to conventional methods. Pharmaceutically acceptable esters can be appended onto hydroxy groups by reaction of the compound that contains the hydroxy group with acid and an alkylcarboxylic acid such as acetic acid, or with acid and an arylcarboxylic acid such as benzoic acid. In the case of compounds containing carboxylic acid groups, the pharmaceutically acceptable esters are prepared from compounds containing the carboxylic acid groups by reaction of the compound with base such as triethylamine and an alkyl halide, for example with methyl iodide, benzyl iodide, cyclopentyl iodide or alkyl triflate. They also can be prepared by reaction of the compound with an acid such as hydrochloric acid and an alcohol such as ethanol or methanol.

The term “pharmaceutically acceptable amide” refers to non-toxic amides of the present disclosure derived from ammonia, primary C 1-to-C 6 alkyl amines and secondary C 1-to-C 6 dialkyl amines. In the case of secondary amines, the amine can also be in the form of a 5- or 6-membered heterocycle containing one nitrogen atom. Amides derived from ammonia, C 1-to-C 3 alkyl primary amides and C 1-to-C 2 dialkyl secondary amides are preferred. Amides of disclosed compounds can be prepared according to conventional methods. Pharmaceutically acceptable amides can be prepared from compounds containing primary or secondary amine groups by reaction of the compound that contains the amino group with an alkyl anhydride, aryl anhydride, acyl halide, or aroyl halide. In the case of compounds containing carboxylic acid groups, the pharmaceutically acceptable amides are prepared from compounds containing the carboxylic acid groups by reaction of the compound with base such as triethylamine, a dehydrating agent such as dicyclohexyl carbodiimide or carbonyl diimidazole, and an alkyl amine, dialkylamine, for example with methylamine, diethylamine, and piperidine. They also can be prepared by reaction of the compound with an acid such as sulfuric acid and an alkylcarboxylic acid such as acetic acid, or with acid and an arylcarboxylic acid such as benzoic acid under dehydrating conditions such as with molecular sieves added. The composition can contain a compound of the present disclosure in the form of a pharmaceutically acceptable prodrug.

The term “pharmaceutically acceptable prodrug” or “prodrug” represents those prodrugs of the compounds of the present disclosure which are, within the scope of sound medical judgment, suitable for use in contact with the tissues of humans and lower animals without undue toxicity, irritation, allergic response, and the like, commensurate with a reasonable benefit/risk ratio, and effective for their intended use. Prodrugs of the present disclosure can be rapidly transformed in vivo to a parent compound having a structure of a disclosed compound, for example, by hydrolysis in blood. A thorough discussion is provided in T. Higuchi and V. Stella, Pro-drugs as Novel Delivery Systems, V. 14 of the A.C.S. Symposium Series, and in Edward B. Roche, ed., Bioreversible Carriers in Drug Design, American Pharmaceutical Association and Pergamon Press (1987).

As used herein, the term “derivative” refers to a compound having a structure derived from the structure of a parent compound (e.g., a compound disclosed herein) and whose structure is sufficiently similar to those disclosed herein and based upon that similarity, would be expected by one skilled in the art to exhibit the same or similar activities and utilities as the claimed compounds, or to induce, as a precursor, the same or similar activities and utilities as the claimed compounds. Exemplary derivatives include salts, esters, amides, salts of esters or amides, and N-oxides of a parent compound.

The term “contacting” as used herein refers to bringing a disclosed compound or pharmaceutical composition in proximity to a cell, a target protein, or other biological entity together in such a manner that the disclosed compound or pharmaceutical composition can affect the activity of the a cell, target protein, or other biological entity, either directly; i.e., by interacting with the cell, target protein, or other biological entity itself, or indirectly; i.e., by interacting with another molecule, co-factor, factor, or protein on which the activity of the cell, target protein, or other biological entity itself is dependent.

As used herein, nomenclature for compounds, including organic compounds, can be given using common names, IUPAC, IUBMB, or CAS recommendations for nomenclature. When one or more stereochemical features are present, Cahn-Ingold-Prelog rules for stereochemistry can be employed to designate stereochemical priority, E/Z specification, and the like. One of skill in the art can readily ascertain the structure of a compound if given a name, either by systemic reduction of the compound structure using naming conventions, or by commercially available software, such as CHEMDRAW™ (Cambridgesoft Corporation, U.S.A.).

It is understood, that unless otherwise specified, temperatures referred to herein are based on atmospheric pressure (i.e. one atmosphere).

Described herein are 1,3,4-triazolyl pyridine compounds and analogs that have therapeutic or clinical utility. Also described herein are methods of synthesizing the compounds and analogs Also described herein are methods of administering the compounds and/or analogs to a subject in need thereof. In some aspects, the subject can have tuberculosis. Other compositions, compounds, methods, features, and advantages of the present disclosure will be or become apparent to one having ordinary skill in the art upon examination of the following drawings, detailed description, and examples. It is intended that all such additional compositions, compounds, methods, features, and advantages be included within this description, and be within the scope of the present disclosure.

### Pharmaceutical Compositions

In various aspects, the present disclosure relates to pharmaceutical compositions comprising a therapeutically effective amount of at least one disclosed compound, at least one product of a disclosed method, or a pharmaceutically acceptable salt thereof. As used herein, “pharmaceutically-acceptable carriers” means one or more of a pharmaceutically acceptable diluents, preservatives, antioxidants, solubilizers, emulsifiers, coloring agents, releasing agents, coating agents, sweetening, flavoring and perfuming agents, and adjuvants. The disclosed pharmaceutical compositions can be conveniently presented in unit dosage form and prepared by any of the methods well known in the art of pharmacy and pharmaceutical sciences.

In a further aspect, the disclosed pharmaceutical compositions comprise a therapeutically effective amount of at least one disclosed compound, at least one product of a disclosed method, or a pharmaceutically acceptable salt thereof as an active ingredient, a pharmaceutically acceptable carrier, optionally one or more other therapeutic agent, and optionally one or more adjuvant. The disclosed pharmaceutical compositions include those suitable for oral, rectal, topical, pulmonary, nasal, and parenteral administration, although the most suitable route in any given case will depend on the particular host, and nature and severity of the conditions for which the active ingredient is being administered. In a further aspect, the disclosed pharmaceutical composition can be formulated to allow administration orally, nasally, via inhalation, parenterally, transmucosally, intramuscularly, intravenously, or intraventricularly.

In one aspect, the pharmaceutical formulations including the disclosed compounds can be prepared as inhaled forms. In another aspect, the pharmaceutical formulations can be dispensed by metered-dose inhalers with or without adapter chambers, dry powder inhalers, nebulizers used with or without masks, sprays, or soft mist inhalers. In some aspects, when the pharmaceutical formulations are inhaled, they can include excipients especially useful for inhaled drugs including, but not limited to, sugars including lactose monohydrate or anhydrous lactose, lipids including oleic acid, amino acids, surfactants including lecithin, polymers, absorption enhancers, propellants, solvents, and the like. In some aspects, the pharmaceutical formulations can be micronized or spray dried or processed by another means prior to loading into dispensing devices. In another aspect, those skilled in the art will be able to select excipients to achieve the desired particle size, delivered dose, flowability, and other properties.

In one aspect, delivered dosages from aerosol or inhaled forms of the pharmaceutical compositions disclosed herein can be approximately the same as dosages for other forms (e.g., oral, intramuscular injection). In an alternative aspect, delivered dosages from aerosol or inhaled forms of the pharmaceutical compositions disclosed herein can be higher than dosages for other forms, due to lack of mobility to other organs and systems. In some aspects, aerosol and/or inhaled forms of the pharmaceutical compositions can be as high as 30-35 mg/kg of subject body weight or greater. In another aspect, dosages for aerosol and/or inhaled forms of the pharmaceutical compositions can be less than 30 mg/kg of subject body weight. Without wishing to be bound by theory, delivery of the disclosed compounds directly to the lungs and airways by inhalation may reduce the dose of medication required to exert a protective effect on the airways due to direct contact of the disclosed compounds and/or any other active ingredients with the affected cells.

In another aspect, the aerosol and/or inhaled forms of the pharmaceutical compositions disclosed herein can be used once per day, or twice per day, or as needed as symptoms dictate. In a further aspect, the aerosol and/or inhaled forms of the pharmaceutical compositions disclosed herein can be used as single treatments or on a consistent basis until symptoms subside. In some aspects, aerosol and/or inhaled forms can be co-administered with oral or parenteral therapies.

As used herein, “parenteral administration” includes administration by bolus injection or infusion, as well as administration by intravenous, intramuscular, intraarterial, intrathecal, intracapsular, intraorbital, intracardiac, intradermal, intraperitoneal, transtracheal, subcutaneous, subcuticular, intraarticular, subcapsular subarachnoid, intraspinal, epidural and intrasternal injection and infusion.

In one aspect, a parenterally-administered or an intravenously-administered compound can include microparticles and/or nanoparticles. In a further aspect, the microparticles and/or nanoparticles can include a gel phase or hydrogel phase (i.e., can be microgels and/or nanogels). In a further aspect, the microgels and/or nanogels can include one or more of chitosan, polyethylene glycol, poly(lactic-co-glycolic acid), polystyrene, poly(methacrylic acid), albumin, macroaggregated albumin, or any combination thereof.

In various aspects, the present disclosure also relates to a pharmaceutical composition comprising a pharmaceutically acceptable carrier or diluent and, as active ingredient, a therapeutically effective amount of a disclosed compound, a product of a disclosed method of making, a pharmaceutically acceptable salt, a hydrate thereof, a solvate thereof, a polymorph thereof, or a stereochemically isomeric form thereof. In a further aspect, a disclosed compound, a product of a disclosed method of making, a pharmaceutically acceptable salt, a hydrate thereof, a solvate thereof, a polymorph thereof, or a stereochemically isomeric form thereof, or any subgroup or combination thereof may be formulated into various pharmaceutical forms for administration purposes.

Pharmaceutically acceptable salts can be prepared from pharmaceutically acceptable non-toxic bases or acids. For therapeutic use, salts of the disclosed compounds are those wherein the counter ion is pharmaceutically acceptable. However, salts of acids and bases which are non-pharmaceutically acceptable may also find use, for example, in the preparation or purification of a pharmaceutically acceptable compound. All salts, whether pharmaceutically acceptable or not, are contemplated by the present disclosure. Pharmaceutically acceptable acid and base addition salts are meant to comprise the therapeutically active non-toxic acid and base addition salt forms which the disclosed compounds are able to form.

In various aspects, a disclosed compound comprising an acidic group or moiety, e.g., a carboxylic acid group, can be used to prepare a pharmaceutically acceptable salt. For example, such a disclosed compound may comprise an isolation step comprising treatment with a suitable inorganic or organic base. In some cases, it may be desirable in practice to initially isolate a compound from the reaction mixture as a pharmaceutically unacceptable salt and then simply convert the latter back to the free acid compound by treatment with an acidic reagent, and subsequently convert the free acid to a pharmaceutically acceptable base addition salt. These base addition salts can be readily prepared using conventional techniques, e.g., by treating the corresponding acidic compounds with an aqueous solution containing the desired pharmacologically acceptable cations and then evaporating the resulting solution to dryness, preferably under reduced pressure. Alternatively, they also can be prepared by mixing lower alkanolic solutions of the acidic compounds and the desired alkali metal alkoxide together, and then evaporating the resulting solution to dryness in the same manner as before.

Bases which can be used to prepare the pharmaceutically acceptable base-addition salts of the base compounds are those which can form non-toxic base-addition salts, i.e., salts containing pharmacologically acceptable cations such as, alkali metal cations (e.g., lithium, potassium and sodium), alkaline earth metal cations (e.g., calcium and magnesium), ammonium or other water-soluble amine addition salts such as N-methylglucamine-(meglumine), lower alkanolammonium and other such bases of organic amines. In a further aspect, derived from pharmaceutically acceptable organic non-toxic bases include primary, secondary, and tertiary amines, as well as cyclic amines and substituted amines such as naturally occurring and synthesized substituted amines. In various aspects, such pharmaceutically acceptable organic non-toxic bases include, but are not limited to, ammonia, methylamine, ethylamine, propylamine, isopropylamine, any of the four butylamine isomers, betaine, caffeine, choline, dimethylamine, diethylamine, diethanolamine, dipropylamine, diisopropylamine, di-n-butylamine, N,N′-dibenzylethylenediamine, pyrrolidine, piperidine, morpholine, trimethylamine, triethylamine, tripropylamine, tromethamine, 2-diethylaminoethanol, 2-dimethylaminoethanol, ethanolamine, quinuclidine, pyridine, quinoline and isoquinoline; benzathine, N-methyl-D-glucamine, ethylenediamine, N-ethylmorpholine, N-ethylpiperidine, glucamine, glucosamine, methylglucamine, morpholine, piperazine, piperidine, polyamine resins, procaine, purines, theobromine, hydrabamine salts, and salts with amino acids such as, for example, histidine, arginine, lysine and the like. The foregoing salt forms can be converted by treatment with acid back into the free acid form.

In various aspects, a disclosed compound comprising a protonatable group or moiety, e.g., an amino group, can be used to prepare a pharmaceutically acceptable salt. For example, such a disclosed compound may comprise an isolation step comprising treatment with a suitable inorganic or organic acid. In some cases, it may be desirable in practice to initially isolate a compound from the reaction mixture as a pharmaceutically unacceptable salt and then simply convert the latter back to the free base compound by treatment with a basic reagent, and subsequently convert the free base to a pharmaceutically acceptable acid addition salt. These acid addition salts can be readily prepared using conventional techniques, e.g., by treating the corresponding basic compounds with an aqueous solution containing the desired pharmacologically acceptable anions and then evaporating the resulting solution to dryness, preferably under reduced pressure. Alternatively, they also can be prepared by treating the free base form of the disclosed compound with a suitable pharmaceutically acceptable non-toxic inorganic or organic acid.

Acids that can be used to prepare the pharmaceutically acceptable acid-addition salts of the base compounds are those which can form non-toxic acid-addition salts, i.e., salts containing pharmacologically acceptable anions formed from their corresponding inorganic and organic acids. Exemplary, but non-limiting, inorganic acids include hydrochloric hydrobromic, sulfuric, nitric, phosphoric and the like. Exemplary, but non-limiting, organic acids include acetic, benzenesulfonic, benzoic, camphorsulfonic, citric, ethanesulfonic, fumaric, gluconic, glutamic, isethionic, lactic, maleic, malic, mandelicmethanesulfonic, mucic, pamoic, pantothenic, succinic, tartaric, p-toluenesulfonic acid and the like. In a further aspect, the acid-addition salt comprises an anion formed from hydrobromic, hydrochloric, maleic, phosphoric, sulfuric, and tartaric acids.

In practice, the compounds of the present disclosure, or pharmaceutically acceptable salts thereof, of the present disclosure can be combined as the active ingredient in intimate admixture with a pharmaceutical carrier according to conventional pharmaceutical compounding techniques.

The carrier can take a wide variety of forms depending on the form of preparation desired for administration, e.g., oral or parenteral (including intravenous). Thus, the pharmaceutical compositions of the present disclosure can be presented as discrete units suitable for oral administration such as capsules, cachets, or tablets each containing a predetermined amount of the active ingredient. Further, the compositions can be presented as a powder, as granules, as a solution, as a suspension in an aqueous liquid, as a non-aqueous liquid, as an oil-in-water emulsion or as a water-in-oil liquid emulsion. In addition to the common dosage forms set out above, the compounds of the present disclosure, and/or pharmaceutically acceptable salt(s) thereof, can also be administered by controlled release means and/or delivery devices. The compositions can be prepared by any of the methods of pharmacy. In general, such methods include a step of bringing into association the active ingredient with the carrier that constitutes one or more necessary ingredients. In general, the compositions are prepared by uniformly and intimately admixing the active ingredient with liquid carriers or finely divided solid carriers or both. The product can then be conveniently shaped into the desired presentation.

It is especially advantageous to formulate the aforementioned pharmaceutical compositions in unit dosage form for ease of administration and uniformity of dosage. The term “unit dosage form,” as used herein, refers to physically discrete units suitable as unitary dosages, each unit containing a predetermined quantity of active ingredient calculated to produce the desired therapeutic effect in association with the required pharmaceutical carrier. That is, a “unit dosage form” is taken to mean a single dose wherein all active and inactive ingredients are combined in a suitable system, such that the patient or person administering the drug to the patient can open a single container or package with the entire dose contained therein, and does not have to mix any components together from two or more containers or packages. Typical examples of unit dosage forms are tablets (including scored or coated tablets), capsules or pills for oral administration; single dose vials for injectable solutions or suspension; suppositories for rectal administration; powder packets; wafers; and segregated multiples thereof. This list of unit dosage forms is not intended to be limiting in any way, but merely to represent typical examples of unit dosage forms.

The pharmaceutical compositions disclosed herein comprise a compound of the present disclosure (or pharmaceutically acceptable salts thereof) as an active ingredient, a pharmaceutically acceptable carrier, and optionally one or more additional therapeutic agents. In various aspects, the disclosed pharmaceutical compositions can include a pharmaceutically acceptable carrier and a disclosed compound, or a pharmaceutically acceptable salt thereof. In a further aspect, a disclosed compound, or pharmaceutically acceptable salt thereof, can also be included in a pharmaceutical composition in combination with one or more other therapeutically active compounds. The instant compositions include compositions suitable for oral, rectal, topical, and parenteral (including subcutaneous, intramuscular, and intravenous) administration, although the most suitable route in any given case will depend on the particular host, and nature and severity of the conditions for which the active ingredient is being administered. The pharmaceutical compositions can be conveniently presented in unit dosage form and prepared by any of the methods well known in the art of pharmacy.

Techniques and compositions for making dosage forms useful for materials and methods described herein are described, for example, in the following references: Modern Pharmaceutics, Chapters 9 and 10 (Banker & Rhodes, Editors, 1979); Pharmaceutical Dosage Forms: Tablets (Lieberman et al., 1981); Ansel, Introduction to Pharmaceutical Dosage Forms 2nd Edition (1976); Remington's Pharmaceutical Sciences, 17th ed. (Mack Publishing Company, Easton, Pa., 1985); Advances in Pharmaceutical Sciences (David Ganderton, Trevor Jones, Eds., 1992); Advances in Pharmaceutical Sciences Vol 7. (David Ganderton, Trevor Jones, James McGinity, Eds., 1995); Aqueous Polymeric Coatings for Pharmaceutical Dosage Forms (Drugs and the Pharmaceutical Sciences, Series 36 (James McGinity, Ed., 1989); Pharmaceutical Particulate Carriers: Therapeutic Applications: Drugs and the Pharmaceutical Sciences, Vol 61 (Alain Rolland, Ed., 1993); Drug Delivery to the Gastrointestinal Tract (Ellis Horwood Books in the Biological Sciences. Series in Pharmaceutical Technology; J. G. Hardy, S. S. Davis, Clive G. Wilson, Eds.); Modern Pharmaceutics Drugs and the Pharmaceutical Sciences, Vol 40 (Gilbert S. Banker, Christopher T. Rhodes, Eds.).

The compounds described herein are typically to be administered in admixture with suitable pharmaceutical diluents, excipients, extenders, or carriers (termed herein as a pharmaceutically acceptable carrier, or a carrier) suitably selected with respect to the intended form of administration and as consistent with conventional pharmaceutical practices. The deliverable compound will be in a form suitable for oral, rectal, topical, intravenous injection or parenteral administration. Carriers include solids or liquids, and the type of carrier is chosen based on the type of administration being used. The compounds may be administered as a dosage that has a known quantity of the compound.

Because of the ease in administration, oral administration can be a preferred dosage form, and tablets and capsules represent the most advantageous oral dosage unit forms in which case solid pharmaceutical carriers are obviously employed. However, other dosage forms may be suitable depending upon clinical population (e.g., age and severity of clinical condition), solubility properties of the specific disclosed compound used, and the like. Accordingly, the disclosed compounds can be used in oral dosage forms such as pills, powders, granules, elixirs, tinctures, suspensions, syrups, and emulsions. In preparing the compositions for oral dosage form, any convenient pharmaceutical media can be employed. For example, water, glycols, oils, alcohols, flavoring agents, preservatives, coloring agents and the like can be used to form oral liquid preparations such as suspensions, elixirs, and solutions; while carriers such as starches, sugars, microcrystalline cellulose, diluents, granulating agents, lubricants, binders, disintegrating agents, and the like can be used to form oral solid preparations such as powders, capsules, and tablets. Because of their ease of administration, tablets and capsules are the preferred oral dosage units whereby solid pharmaceutical carriers are employed. Optionally, tablets can be coated by standard aqueous or nonaqueous techniques.

The disclosed pharmaceutical compositions in an oral dosage form can comprise one or more pharmaceutical excipient and/or additive. Non-limiting examples of suitable excipients and additives include gelatin, natural sugars such as raw sugar or lactose, lecithin, pectin, starches (for example corn starch or amylose), dextran, polyvinyl pyrrolidone, polyvinyl acetate, gum arabic, alginic acid, tylose, talcum, lycopodium, silica gel (for example colloidal), cellulose, cellulose derivatives (for example cellulose ethers in which the cellulose hydroxy groups are partially etherified with lower saturated aliphatic alcohols and/or lower saturated, aliphatic oxyalcohols, for example methyl oxypropyl cellulose, methyl cellulose, hydroxypropyl methyl cellulose, hydroxypropyl methyl cellulose phthalate), fatty acids as well as magnesium, calcium or aluminum salts of fatty acids with 12 to 22 carbon atoms, in particular saturated (for example stearates), emulsifiers, oils and fats, in particular vegetable (for example, peanut oil, castor oil, olive oil, sesame oil, cottonseed oil, corn oil, wheat germ oil, sunflower seed oil, cod liver oil, in each case also optionally hydrated); glycerol esters and polyglycerol esters of saturated fatty acids C₁₂H₂₄O₂ to C₁₈H₃₆O₂ and their mixtures, it being possible for the glycerol hydroxy groups to be totally or also only partly esterified (for example mono-, di- and triglycerides); pharmaceutically acceptable mono- or multivalent alcohols and polyglycols such as polyethylene glycol and derivatives thereof, esters of aliphatic saturated or unsaturated fatty acids (2 to 22 carbon atoms, in particular 10-18 carbon atoms) with monovalent aliphatic alcohols (1 to 20 carbon atoms) or multivalent alcohols such as glycols, glycerol, diethylene glycol, pentacrythritol, sorbitol, mannitol and the like, which may optionally also be etherified, esters of citric acid with primary alcohols, acetic acid, urea, benzyl benzoate, dioxolanes, glyceroformals, tetrahydrofurfuryl alcohol, polyglycol ethers with C1-C12-alcohols, dimethylacetamide, lactamides, lactates, ethylcarbonates, silicones (in particular medium-viscous polydimethyl siloxanes), calcium carbonate, sodium carbonate, calcium phosphate, sodium phosphate, magnesium carbonate and the like.

Other auxiliary substances useful in preparing an oral dosage form are those which cause disintegration (so-called disintegrants), such as: cross-linked polyvinyl pyrrolidone, sodium carboxymethyl starch, sodium carboxymethyl cellulose or microcrystalline cellulose. Conventional coating substances may also be used to produce the oral dosage form. Those that may for example be considered are: polymerizates as well as copolymerizates of acrylic acid and/or methacrylic acid and/or their esters; copolymerizates of acrylic and methacrylic acid esters with a lower ammonium group content (for example EudragitR RS), copolymerizates of acrylic and methacrylic acid esters and trimethyl ammonium methacrylate (for example EudragitR RL); polyvinyl acetate; fats, oils, waxes, fatty alcohols; hydroxypropyl methyl cellulose phthalate or acetate succinate; cellulose acetate phthalate, starch acetate phthalate as well as polyvinyl acetate phthalate, carboxy methyl cellulose; methyl cellulose phthalate, methyl cellulose succinate, -phthalate succinate as well as methyl cellulose phthalic acid half ester; zein; ethyl cellulose as well as ethyl cellulose succinate; shellac, gluten; ethylcarboxyethyl cellulose; ethacrylate-maleic acid anhydride copolymer; maleic acid anhydride-vinyl methyl ether copolymer; styrol-maleic acid copolymerizate; 2-ethyl-hexyl-acrylate maleic acid anhydride; crotonic acid-vinyl acetate copolymer; glutaminic acid/glutamic acid ester copolymer; carboxymethylethylcellulose glycerol monooctanoate; cellulose acetate succinate; polyarginine.

Plasticizing agents that may be considered as coating substances in the disclosed oral dosage forms are: citric and tartaric acid esters (acetyl-triethyl citrate, acetyl tributyl-, tributyl-, triethyl-citrate); glycerol and glycerol esters (glycerol diacetate, -triacetate, acetylated monoglycerides, castor oil); phthalic acid esters (dibutyl-, diamyl-, diethyl-, dimethyl-, dipropyl-phthalate), di-(2-methoxy- or 2-ethoxyethyl)-phthalate, ethylphthalyl glycolate, butylphthalylethyl glycolate and butylglycolate; alcohols (propylene glycol, polyethylene glycol of various chain lengths), adipates (diethyladipate, di-(2-methoxy- or 2-ethoxyethyl)-adipate; benzophenone; diethyl- and diburylsebacate, dibutylsuccinate, dibutyltartrate; diethylene glycol dipropionate; ethyleneglycol diacetate, -dibutyrate, -dipropionate; tributyl phosphate, tributyrin; polyethylene glycol sorbitan monooleate (polysorbates such as Polysorbar 50); sorbitan monooleate.

Moreover, suitable binders, lubricants, disintegrating agents, coloring agents, flavoring agents, flow-inducing agents, and melting agents may be included as carriers. The pharmaceutical carrier employed can be, for example, a solid, liquid, or gas. Examples of solid carriers include, but are not limited to, lactose, terra alba, sucrose, glucose, methylcellulose, dicalcium phosphate, calcium sulfate, mannitol, sorbitol talc, starch, gelatin, agar, pectin, acacia, magnesium stearate, and stearic acid. Examples of liquid carriers are sugar syrup, peanut oil, olive oil, and water. Examples of gaseous carriers include carbon dioxide and nitrogen.

In various aspects, a binder can include, for example, starch, gelatin, natural sugars such as glucose or beta-lactose, corn sweeteners, natural and synthetic gums such as acacia, tragacanth, or sodium alginate, carboxymethylcellulose, polyethylene glycol, waxes, and the like. Lubricants used in these dosage forms include sodium oleate, sodium stearate, magnesium stearate, sodium benzoate, sodium acetate, sodium chloride, and the like. In a further aspect, a disintegrator can include, for example, starch, methyl cellulose, agar, bentonite, xanthan gum, and the like.

In various aspects, an oral dosage form, such as a solid dosage form, can comprise a disclosed compound that is attached to polymers as targetable drug carriers or as a prodrug. Suitable biodegradable polymers useful in achieving controlled release of a drug include, for example, polylactic acid, polyglycolic acid, copolymers of polylactic and polyglycolic acid, caprolactones, polyhydroxy butyric acid, polyorthoesters, polyacetals, polydihydropyrans, polycyanoacylates, and hydrogels, preferably covalently crosslinked hydrogels.

Tablets may contain the active ingredient in admixture with non-toxic pharmaceutically acceptable excipients which are suitable for the manufacture of tablets. These excipients may be, for example, inert diluents, such as calcium carbonate, sodium carbonate, lactose, calcium phosphate or sodium phosphate; granulating and disintegrating agents, for example, corn starch, or alginic acid; binding agents, for example starch, gelatin or acacia, and lubricating agents, for example magnesium stearate, stearic acid, or talc. The tablets may be uncoated or they may be coated by known techniques to delay disintegration and absorption in the gastrointestinal tract and thereby provide a sustained action over a longer period.

A tablet containing a disclosed compound can be prepared by compression or molding, optionally with one or more accessory ingredients or adjuvants. Compressed tablets can be prepared by compressing, in a suitable machine, the active ingredient in a free-flowing form such as powder or granules, optionally mixed with a binder, lubricant, inert diluent, surface active or dispersing agent. Molded tablets can be made by molding in a suitable machine, a mixture of the powdered compound moistened with an inert liquid diluent.

In various aspects, a solid oral dosage form, such as a tablet, can be coated with an enteric coating to prevent ready decomposition in the stomach. In various aspects, enteric coating agents include, but are not limited to, hydroxypropylmethylcellulose phthalate, methacrylic acid-methacrylic acid ester copolymer, polyvinyl acetate-phthalate, and cellulose acetate phthalate. Akihiko Hasegawa “Application of solid dispersions of Nifedipine with enteric coating agent to prepare a sustained-release dosage form” Chem. Pharm. Bull. 33:1615-1619 (1985). Various enteric coating materials may be selected on the basis of testing to achieve an enteric coated dosage form designed ab initio to have a preferable combination of dissolution time, coating thicknesses and diametral crushing strength (e.g., see S. C. Porter et al. “The Properties of Enteric Tablet Coatings Made From Polyvinyl Acetate-phthalate and Cellulose acetate Phthalate,” J. Pharm. Pharmacol. 22:42p (1970)). In a further aspect, the enteric coating may comprise hydroxypropyl-methylcellulose phthalate, methacrylic acid-methacrylic acid ester copolymer, polyvinyl acetate-phthalate, and cellulose acetate phthalate.

In various aspects, an oral dosage form can be a solid dispersion with a water soluble or a water insoluble carrier. Examples of water soluble or water insoluble carrier include, but are not limited to, polyethylene glycol, polyvinylpyrrolidone, hydroxypropylmethyl-cellulose, phosphatidylcholine, polyoxyethylene hydrogenated castor oil, hydroxypropylmethylcellulose phthalate, carboxymethylethylcellulose, or hydroxypropylmethylcellulose, ethyl cellulose, or stearic acid.

In various aspects, an oral dosage form can be in a liquid dosage form, including those that are ingested, or alternatively, administered as a mouth wash or gargle. For example, a liquid dosage form can include aqueous suspensions, which contain the active materials in admixture with excipients suitable for the manufacture of aqueous suspensions. In addition, oily suspensions may be formulated by suspending the active ingredient in a vegetable oil, for example arachis oil, olive oil, sesame oil or coconut oil, or in a mineral oil such as liquid paraffin. Oily suspensions may also contain various excipients. The pharmaceutical compositions of the present disclosure may also be in the form of oil-in-water emulsions, which may also contain excipients such as sweetening and flavoring agents.

For the preparation of solutions or suspensions it is, for example, possible to use water, particularly sterile water, or physiologically acceptable organic solvents, such as alcohols (ethanol, propanol, isopropanol, 1,2-propylene glycol, polyglycols and their derivatives, fatty alcohols, partial esters of glycerol), oils (for example peanut oil, olive oil, sesame oil, almond oil, sunflower oil, soya bean oil, castor oil, bovine hoof oil), paraffins, dimethyl sulfoxide, triglycerides and the like.

In the case of a liquid dosage form such as a drinkable solutions, the following substances may be used as stabilizers or solubilizers: lower aliphatic mono- and multivalent alcohols with 2-4 carbon atoms, such as ethanol, n-propanol, glycerol, polyethylene glycols with molecular weights between 200-600 (for example 1 to 40% aqueous solution), diethylene glycol monoethyl ether, 1,2-propylene glycol, organic amides, for example amides of aliphatic C1-C6-carboxylic acids with ammonia or primary, secondary or tertiary C1-C4-amines or C1-C4-hydroxy amines such as urea, urethane, acetamide, N-methyl acetamide, N,N-diethyl acetamide, N,N-dimethyl acetamide, lower aliphatic amines and diamines with 2-6 carbon atoms, such as ethylene diamine, hydroxyethyl theophylline, tromethamine (for example as 0.1 to 20% aqueous solution), aliphatic amino acids.

In preparing the disclosed liquid dosage form can comprise solubilizers and emulsifiers such as the following non-limiting examples can be used: polyvinyl pyrrolidone, sorbitan fatty acid esters such as sorbitan trioleate, phosphatides such as lecithin, acacia, tragacanth, polyoxyethylated sorbitan monooleate and other ethoxylated fatty acid esters of sorbitan, polyoxyethylated fats, polyoxyethylated oleotriglycerides, linolizated oleotriglycerides, polyethylene oxide condensation products of fatty alcohols, alkylphenols or fatty acids or also 1-methyl-3-(2-hydroxyethyl)imidazolidone-(2). In this context, polyoxyethylated means that the substances in question contain polyoxyethylene chains, the degree of polymerization of which generally lies between 2 and 40 and in particular between 10 and 20. Polyoxyethylated substances of this kind may for example be obtained by reaction of hydroxyl group-containing compounds (for example mono- or diglycerides or unsaturated compounds such as those containing oleic acid radicals) with ethylene oxide (for example 40 Mol ethylene oxide per 1 Mol glyceride). Examples of oleotriglycerides are olive oil, peanut oil, castor oil, sesame oil, cottonseed oil, corn oil. See also Dr. H. P. Fiedler “Lexikon der Hillsstoffe für Pharmazie, Kostnetik und angrenzende Gebiete” 1971, pages 191-195.

In various aspects, a liquid dosage form can further comprise preservatives, stabilizers, buffer substances, flavor correcting agents, sweeteners, colorants, antioxidants, and complex formers and the like. Complex formers which may be for example be considered are: chelate formers such as ethylene diamine retrascetic acid, nitrilotriacetic acid, diethylene triamine pentacetic acid and their salts.

It may optionally be necessary to stabilize a liquid dosage form with physiologically acceptable bases or buffers to a pH range of approximately 6 to 9. Preference may be given to as neutral or weakly basic a pH value as possible (up to pH 8).

In order to enhance the solubility and/or the stability of a disclosed compound in a disclosed liquid dosage form, a parenteral injection form, or an intravenous injectable form, it can be advantageous to employ α-, β- or γ-cyclodextrins or their derivatives, in particular hydroxyalkyl substituted cyclodextrins, e.g. 2-hydroxypropyl-β-cyclodextrin or sulfobutyl-β-cyclodextrin. Also co-solvents such as alcohols may improve the solubility and/or the stability of the compounds according to the present disclosure in pharmaceutical compositions.

In various aspects, a disclosed liquid dosage form, a parenteral injection form, or an intravenous injectable form can further comprise liposome delivery systems, such as small unilamellar vesicles, large unilamellar vesicles, and multilamellar vesicles. Liposomes can be formed from a variety of phospholipids, such as cholesterol, stearylamine, or phosphatidylcholines.

Pharmaceutical compositions of the present disclosure suitable injection, such as parenteral administration, such as intravenous, intramuscular, or subcutaneous administration. Pharmaceutical compositions for injection can be prepared as solutions or suspensions of the active compounds in water. A suitable surfactant can be included such as, for example, hydroxypropylcellulose. Dispersions can also be prepared in glycerol, liquid polyethylene glycols, and mixtures thereof in oils. Further, a preservative can be included to prevent the detrimental growth of microorganisms.

Pharmaceutical compositions of the present disclosure suitable for parenteral administration can include sterile aqueous or oleaginous solutions, suspensions, or dispersions. Furthermore, the compositions can be in the form of sterile powders for the extemporaneous preparation of such sterile injectable solutions or dispersions. In some aspects, the final injectable form is sterile and must be effectively fluid for use in a syringe. The pharmaceutical compositions should be stable under the conditions of manufacture and storage; thus, preferably should be preserved against the contaminating action of microorganisms such as bacteria and fungi. The carrier can be a solvent or dispersion medium containing, for example, water, ethanol, polyol (e.g., glycerol, propylene glycol and liquid polyethylene glycol), vegetable oils, and suitable mixtures thereof.

Injectable solutions, for example, can be prepared in which the carrier comprises saline solution, glucose solution or a mixture of saline and glucose solution. Injectable suspensions may also be prepared in which case appropriate liquid carriers, suspending agents and the like may be employed. In some aspects, a disclosed parenteral formulation can comprise about 0.01-0.1 M, e.g. about 0.05 M, phosphate buffer. In a further aspect, a disclosed parenteral formulation can comprise about 0.9% saline.

In various aspects, a disclosed parenteral pharmaceutical composition can comprise pharmaceutically acceptable carriers such as aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include but not limited to water, alcoholic/aqueous solutions, emulsions, or suspensions, including saline and buffered media. Parenteral vehicles can include mannitol, normal serum albumin, sodium chloride solution, Ringer's dextrose, dextrose and sodium chloride, lactated Ringer's, and fixed oils. Intravenous vehicles include fluid and nutrient replenishers, electrolyte replenishers such as those based on Ringer's dextrose, and the like. Preservatives and other additives may also be present, such as, for example, antimicrobials, antioxidants, chelating agents, inert gases and the like. In a further aspect, a disclosed parenteral pharmaceutical composition can comprise may contain minor amounts of additives such as substances that enhance isotonicity and chemical stability, e.g., buffers and preservatives. Also contemplated for injectable pharmaceutical compositions are solid form preparations that are intended to be converted, shortly before use, to liquid form preparations. Furthermore, other adjuvants can be included to render the formulation isotonic with the blood of the subject or patient.

In addition to the pharmaceutical compositions described herein above, the disclosed compounds can also be formulated as a depot preparation. Such long acting formulations can be administered by implantation (e.g., subcutaneously or intramuscularly) or by intramuscular injection. Thus, for example, the compounds can be formulated with suitable polymeric or hydrophobic materials (e.g., as an emulsion in an acceptable oil) or ion exchange resins, or as sparingly soluble derivatives, e.g., as a sparingly soluble salt.

Pharmaceutical compositions containing a compound of the present disclosure, and/or pharmaceutically acceptable salts thereof, can also be prepared in powder or liquid concentrate form.

The pharmaceutical composition (or formulation) may be packaged in a variety of ways. Generally, an article for distribution includes a container that contains the pharmaceutical composition in an appropriate form. Suitable containers are well known to those skilled in the art and include materials such as bottles (plastic and glass), sachets, foil blister packs, and the like. The container may also include a tamper proof assemblage to prevent indiscreet access to the contents of the package. In addition, the container typically has deposited thereon a label that describes the contents of the container and any appropriate warnings or instructions.

The disclosed pharmaceutical compositions may, if desired, be presented in a pack or dispenser device which may contain one or more unit dosage forms containing the active ingredient. The pack may for example comprise metal or plastic foil, such as a blister pack. The pack or dispenser device may be accompanied by instructions for administration. The pack or dispenser may also be accompanied with a notice associated with the container in form prescribed by a governmental agency regulating the manufacture, use, or sale of pharmaceuticals, which notice is reflective of approval by the agency of the form of the drug for human or veterinary administration. Such notice, for example, may be the labeling approved by the U.S. Food and Drug Administration for prescription drugs, or the approved product insert. Pharmaceutical compositions comprising a disclosed compound formulated in a compatible pharmaceutical carrier may also be prepared, placed in an appropriate container, and labeled for treatment of an indicated condition.

The exact dosage and frequency of administration depends on the particular disclosed compound, a product of a disclosed method of making, a pharmaceutically acceptable salt, solvate, or polymorph thereof, a hydrate thereof, a solvate thereof, a polymorph thereof, or a stereochemically isomeric form thereof; the particular condition being treated and the severity of the condition being treated; various factors specific to the medical history of the subject to whom the dosage is administered such as the age; weight, sex, extent of disorder and general physical condition of the particular subject, as well as other medication the individual may be taking; as is well known to those skilled in the art. Furthermore, it is evident that said effective daily amount may be lowered or increased depending on the response of the treated subject and/or depending on the evaluation of the physician prescribing the compounds of the present disclosure.

Depending on the mode of administration, the pharmaceutical composition will comprise from 0.05 to 99% by weight, preferably from 0.1 to 70% by weight, more preferably from 0.1 to 50% by weight of the active ingredient, and, from 1 to 99.95% by weight, preferably from 30 to 99.9% by weight, more preferably from 50 to 99.9% by weight of a pharmaceutically acceptable carrier, all percentages being based on the total weight of the composition.

An appropriate dosage level will generally be about 0.01 to 1000 mg per kg patient body weight per day and can be administered in single or multiple doses. In various aspects, the dosage level will be about 0.1 to about 500 mg/kg per day, about 0.1 to 250 mg/kg per day, or about 0.5 to 100 mg/kg per day. A suitable dosage level can be about 0.01 to 1000 mg/kg per day, about 0.01 to 500 mg/kg per day, about 0.01 to 250 mg/kg per day, about 0.05 to 100 mg/kg per day, or about 0.1 to 50 mg/kg per day. Within this range the dosage can be 0.05 to 0.5, 0.5 to 5.0 or 5.0 to 50 mg/kg per day. For oral administration, the compositions are preferably provided in the form of tablets containing 1.0 to 1000 mg of the active ingredient, particularly 1.0, 5.0, 10, 15, 20, 25, 50, 75, 100, 150, 200, 250, 300, 400, 500, 600, 750, 800, 900 and 1000 mg of the active ingredient for the symptomatic adjustment of the dosage of the patient to be treated. The compound can be administered on a regimen of 1 to 4 times per day, preferably once or twice per day. This dosing regimen can be adjusted to provide the optimal therapeutic response.

Such unit doses as described hereinabove and hereinafter can be administered more than once a day, for example, 2, 3, 4, 5 or 6 times a day. In various aspects, such unit doses can be administered 1 or 2 times per day, so that the total dosage for a 70 kg adult is in the range of 0.001 to about 15 mg per kg weight of subject per administration. In a further aspect, dosage is 0.01 to about 1.5 mg per kg weight of subject per administration, and such therapy can extend for a number of weeks or months, and in some cases, years. It will be understood, however, that the specific dose level for any particular patient will depend on a variety of factors including the activity of the specific compound employed; the age, body weight, general health, sex and diet of the individual being treated; the time and route of administration; the rate of excretion; other drugs that have previously been administered; and the severity of the particular disease undergoing therapy, as is well understood by those of skill in the area.

A typical dosage can be one 1 mg to about 100 mg tablet or 1 mg to about 300 mg taken once a day, or multiple times per day, or one time-release capsule or tablet taken once a day and containing a proportionally higher content of active ingredient. The time-release effect can be obtained by capsule materials that dissolve at different pH values, by capsules that release slowly by osmotic pressure, or by any other known means of controlled release.

It can be necessary to use dosages outside these ranges in some cases as will be apparent to those skilled in the art. Further, it is noted that the clinician or treating physician will know how and when to start, interrupt, adjust, or terminate therapy in conjunction with individual patient response.

The present disclosure is further directed to a method for the manufacture of a medicament for treating tuberculosis, especially MDR-TB and XDR-TB in mammals (e.g., humans) comprising combining one or more disclosed compounds, products, or compositions with a pharmaceutically acceptable carrier or diluent. Thus, in one aspect, the present disclosure further relates to a method for manufacturing a medicament comprising combining at least one disclosed compound or at least one disclosed product with a pharmaceutically acceptable carrier or diluent.

The disclosed pharmaceutical compositions can further comprise other therapeutically active compounds, which are usually applied in the treatment of the above mentioned pathological or clinical conditions.

It is understood that the disclosed compositions can be prepared from the disclosed compounds. It is also understood that the disclosed compositions can be employed in the disclosed methods of using.

As already mentioned, the present disclosure relates to a pharmaceutical composition comprising a therapeutically effective amount of a disclosed compound, a product of a disclosed method of making, a pharmaceutically acceptable salt, a hydrate thereof, a solvate thereof, a polymorph thereof, and a pharmaceutically acceptable carrier. Additionally, the present disclosure relates to a process for preparing such a pharmaceutical composition, characterized in that a pharmaceutically acceptable carrier is intimately mixed with a therapeutically effective amount of a compound according to the present disclosure.

As already mentioned, the present disclosure also relates to a pharmaceutical composition comprising a disclosed compound, a product of a disclosed method of making, a pharmaceutically acceptable salt, a hydrate thereof, a solvate thereof, a polymorph thereof, and one or more other drugs in the treatment, prevention, control, amelioration, or reduction of risk of diseases or conditions for a disclosed compound or the other drugs may have utility as well as to the use of such a composition for the manufacture of a medicament. The present disclosure also relates to a combination of disclosed compound, a product of a disclosed method of making, a pharmaceutically acceptable salt, a hydrate thereof, a solvate thereof, or a polymorph thereof.

The present disclosure also relates to such a combination for use as a medicine. The present disclosure also relates to a product comprising (a) disclosed compound, a product of a disclosed method of making, a pharmaceutically acceptable salt, a hydrate thereof, a solvate thereof, a polymorph thereof, and (b) an additional anti-tuberculosis therapeutic agent, as a combined preparation for simultaneous, separate or sequential use in the treatment or prevention of a condition in a mammal, including a human, the treatment or prevention of which is affected or facilitated by the modulatory effect of the disclosed compound and the additional therapeutic agent. The different drugs of such a combination or product may be combined in a single preparation together with pharmaceutically acceptable carriers or diluents, or they may each be present in a separate preparation together with pharmaceutically acceptable carriers or diluents.

In a further aspect, the present disclosure provides methods of treatment comprising administration of a therapeutically effective amount of a disclosed compound or pharmaceutical composition as disclosed herein above to a subject in need thereof.

### Pharmaceutical Compositions Containing the Compounds and Methods of Treatment

In another aspect, disclosed herein is a pharmaceutical composition containing the disclosed compounds or a pharmaceutically acceptable salt thereof. In some aspects, the pharmaceutical compositions further include at least one excipient. In yet another aspect, the pharmaceutical compositions can be administered to a subject orally, by inhalation, parenterally, intravenously, mucosally, or any combination thereof.

Also disclosed herein are methods for treating a bacterial infection caused by Mtb in a subject, the methods including the step of administering to a subject a therapeutically effective amount of a disclosed compound or pharmaceutical composition. In one aspect, the bacterial infection can be multidrug-resistant tuberculosis, extensively drug-resistant tuberculosis, or drug-sensitive tuberculosis. In another aspect, the subject can be a human.

Now having described the aspects of the present disclosure, in general, the following Examples describe some additional aspects of the present disclosure. While aspects of the present disclosure are described in connection with the following examples and the corresponding text and figures, there is no intent to limit aspects of the present disclosure to this description. On the contrary, the intent is to cover all alternatives, modifications, and equivalents included within the spirit and scope of the present disclosure.

### Aspects

The present disclosure can be described in accordance with the following numbered aspects, which should not be confused with the claims.

Aspect 1. A compound of Formula Ia or Ib or a pharmaceutically acceptable salt thereof,

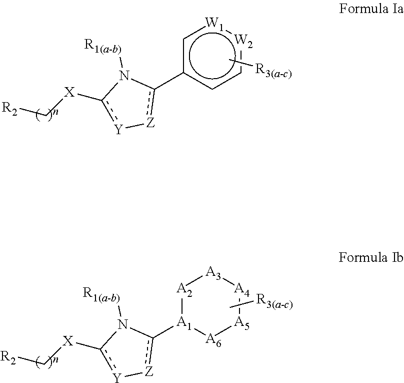

wherein W₁ and W₂ independently comprise N or CR₁₀;
wherein X comprises S, O, NH, NR₄, or CH₂;
wherein Y and Z independently comprise S, O, N, NH, NR₄, CH, or CH₂;
   wherein R₄ comprises hydrogen, halogen, hydroxyl, cyano, nitro, carboxyl, (carboxyl ester)amino, (carboxyl ester)oxy, acyl, formyl; or substituted or unsubstituted C1-C10 alkyl, C3-C7 cycloalkyl, C3-C7 heterocycloalkyl, alkenyl, alkoxy, alkynyl, amino, aminosulfinyl, sulfinyl, sulfonyl, substituted sulfinyloxy, sulfonyloxy, aminosulfonyloxy, aminosulfinyloxy, aminosulfonylamino, acylamino, aminocarbonyloxy, aminocarbonylamino, aminothiocarbonylamino, aminocarbonyl, aminocarbonyl, acyloxy, C3-C7 aryl, or C3-C7 heteroaryl;
wherein n is from 0 to 9;
wherein each of R₁ₐ and R₂ independently comprises hydrogen, halogen, hydroxyl, cyano, nitro, carboxyl, (carboxyl ester)amino, (carboxyl ester)oxy, acyl, formyl; or substituted or unsubstituted C1-C10 alkyl, C3-C7 cycloalkyl, C3-C7 heterocycloalkyl, alkenyl, alkoxy, alkynyl, amino, aminosulfinyl, sulfinyl, sulfonyl, substituted sulfinyloxy, sulfonyloxy, aminosulfonyloxy, aminosulfinyloxy, aminosulfonylamino, acylamino, aminocarbonyloxy, aminocarbonylamino, aminothiocarbonylamino, aminocarbonyl, aminocarbonyl, acyloxy, C3-C7 aryl, or C3-C7 heteroaryl;
wherein each of R_{3a-3c} independently comprises hydrogen, halogen, hydroxyl, cyano, nitro, carboxyl, (carboxyl ester)amino, (carboxyl ester)oxy, acyl, formyl; or substituted or unsubstituted C1-C10 alkyl, C3-C7 cycloalkyl, C3-C7 heterocycloalkyl, alkenyl, alkoxy, alkynyl, amino, aminosulfinyl, sulfinyl, sulfonyl, substituted sulfinyloxy, sulfonyloxy, aminosulfonyloxy, aminosulfinyloxy, aminosulfonylamino, acylamino, aminocarbonyloxy, aminocarbonylamino, aminothiocarbonylamino, aminocarbonyl, aminocarbonyl, acyloxy, C3-C7 aryl, or C3-C7 heteroaryl, or wherein one of R_{3a-3c} is an N, NR₁₁, CH, or CH₂ group connected to an R₁₀ by a substituted or unsubstituted C₁-C₃ alkyl chain;
wherein each R₁₀ independently comprises H or is an N, NR₁₂, CH, or CH₂ group connected to one of R_{3a-3c} by a substituted or unsubstituted C1-C3 alkyl chain;
wherein R₁₁ and R₁₂, if present, independently comprise H or C1-C4 alkyl;
wherein R_{1b} is absent or comprises hydrogen, halogen, hydroxyl, cyano, nitro, carboxyl, (carboxyl ester)amino, (carboxyl ester)oxy, acyl, formyl; or substituted or unsubstituted C1-C10 alkyl, C3-C7 cycloalkyl, C3-C7 heterocycloalkyl, alkenyl, alkoxy, alkynyl, amino, aminosulfinyl, sulfinyl, sulfonyl, substituted sulfinyloxy, sulfonyloxy, aminosulfonyloxy, aminosulfinyloxy, aminosulfonylamino, acylamino, aminocarbonyloxy, aminocarbonylamino, aminothiocarbonylamino, aminocarbonyl, aminocarbonyl, acyloxy, C3-C7 aryl, or C3-C7 heteroaryl; and
wherein A₁-A₆ are independently selected from NR₁₄, CR₁₄, C(R₁₄)₂, N, and O, wherein each R₁₄ is independently H or is one of R_{3a-3c}.

Aspect 2. The compound of aspect 1, wherein W₁ is N, W₂ is CR₁₀, and R₁₀ is H.

Aspect 3. The compound of aspect 1, wherein the compound has a formula

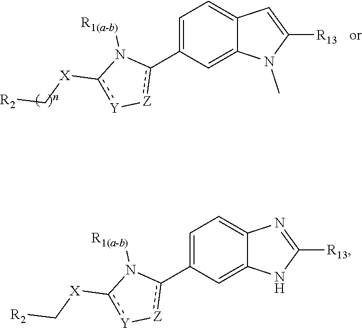

wherein R₁₃ is selected from hydrogen, halogen, hydroxyl, cyano, nitro, carboxyl, (carboxyl ester)amino, (carboxyl ester)oxy, acyl, formyl; or substituted or unsubstituted C1-C10 alkyl, C3-C7 cycloalkyl, C3-C7 heterocycloalkyl, alkenyl, alkoxy, alkynyl, amino, aminosulfinyl, sulfinyl, sulfonyl, substituted sulfinyloxy, sulfonyloxy, aminosulfonyloxy, aminosulfinyloxy, aminosulfonylamino, acylamino, aminocarbonyloxy, aminocarbonylamino, aminothiocarbonylamino, aminocarbonyl, aminocarbonyl, acyloxy, C3-C7 aryl, or C3-C7 heteroaryl.

Aspect 4. The compound of any one of aspects 1-3, wherein R₁₃ is

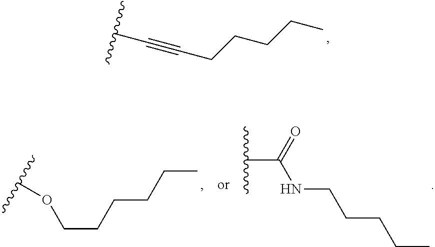

Aspect 5. The compound of any one of aspects 1-4, wherein Y and Z are N and R_{1b} is absent.

Aspect 6. The compound of any one of aspects 1-4, wherein Y and Z are CH and R_{1b} is absent.

Aspect 7. The compound of any one of aspects 1-6, wherein R₁ₐ is methyl, ethyl, cyclopropyl, or —CFH₂.

Aspect 8. The compound of any one of aspects 1-7, wherein R₂ is

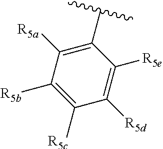

and R₅ₐ-R₅ₑ are independently hydrogen, halogen, cyano, or nitro.

Aspect 9. The compound of aspect 8, wherein R_{5b} and R_{5d} are nitro and R₅ₐ, R_{5c}, and R₅ₑ are hydrogen.

Aspect 10. The compound of aspect 8, wherein R₅ₐ, R_{5b}, R_{5d}, and R₅ₑ, are hydrogen and R_{5c} is chloro or bromo.

Aspect 11. The compound of aspect 8, wherein R₅ₐ, R_{5d}, and R₅ₑ, are hydrogen, R_{5b} is chloro, and R_{5c} is chloro or fluoro.

Aspect 12. The compound of any one of aspects 1-7, wherein R₂ is

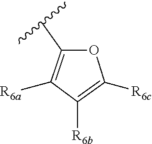

and R₆ₐ-R_{6c} are independently hydrogen or nitro.

Aspect 13. The compound of aspect 12, wherein R_{6c} is nitro and R₆ₐ and R_{6b} are hydrogen.

Aspect 14. The compound of any one of aspects 1-13, wherein n is 0 or 1.

Aspect 15. The compound of any one of aspects 1-14, wherein X is O.

Aspect 16. The compound of any one of aspects 1-14, wherein X is S.

Aspect 17. The compound of any one of aspects 1 or 5-15, wherein A₁-A₆ and R₃ₐ-3c, if present, form a saturated heterocycle selected from:

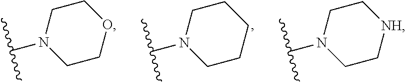 [SMILES: *N1CCOCC1 ; *N1CCCCC1 ; "," ; *N1CCNCC1]

or any combination thereof.

Aspect 18. The compound of any one of aspects 1-17, wherein the compound is

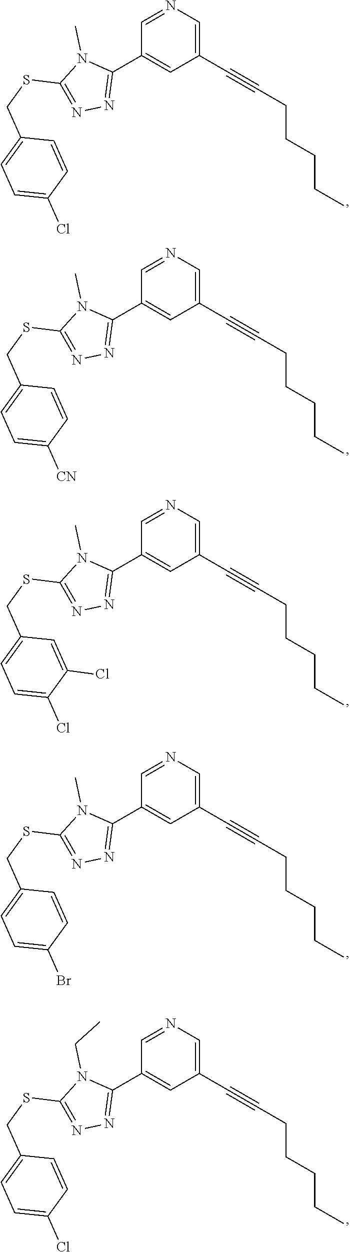 [SMILES: CCCCCC#Cc1cncc(-c2nnc(SCc3ccc(Cl)cc3)n2C)c1 ; CCCCCC#Cc1cncc(-c2nnc(SCc3ccc(C#N)cc3)n2C)c1 ; CCCCCC#Cc1cncc(-c2nnc(SCc3ccc(Cl)c(Cl)c3)n2C)c1 ; CCCCCC#Cc1cncc(-c2nnc(SCc3ccc(Br)cc3)n2C)c1 ; CCCCCC#Cc1cncc(-c2nnc(SCc3ccc(Cl)cc3)n2CC)c1]

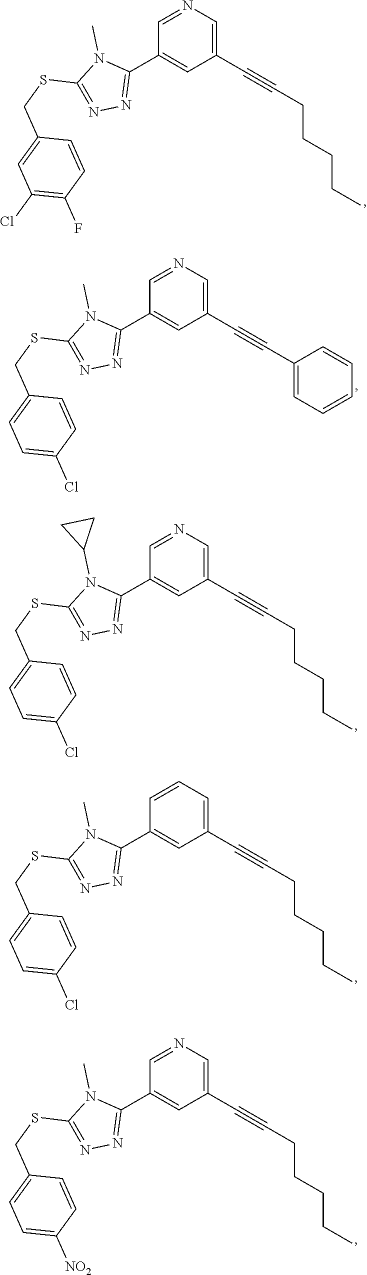

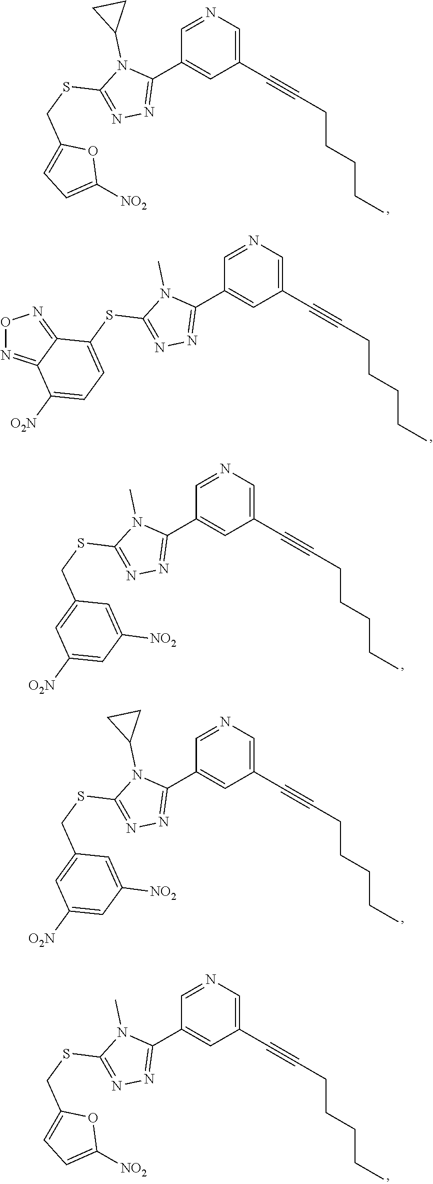 [SMILES: CCCCCC#Cc1cncc(-c2nnc(SCc3ccc([N+](=O)[O-])o3)n2C2CC2)c1 ; CCCCCC#Cc1cncc(-c2nnc(Sc3ccc([N+](=O)[O-])c4nonc34)n2C)c1 ; CCCCCC#Cc1cncc(-c2nnc(SCc3cc([N+](=O)[O-])cc([N+](=O)[O-])c3)n2C)c1 ; CCCCCC#Cc1cncc(-c2nnc(SCc3cc([N+](=O)[O-])cc([N+](=O)[O-])c3)n2C2CC2)c1 ; CCCCCC#Cc1cncc(-c2nnc(SCc3ccc([N+](=O)[O-])o3)n2C)c1]

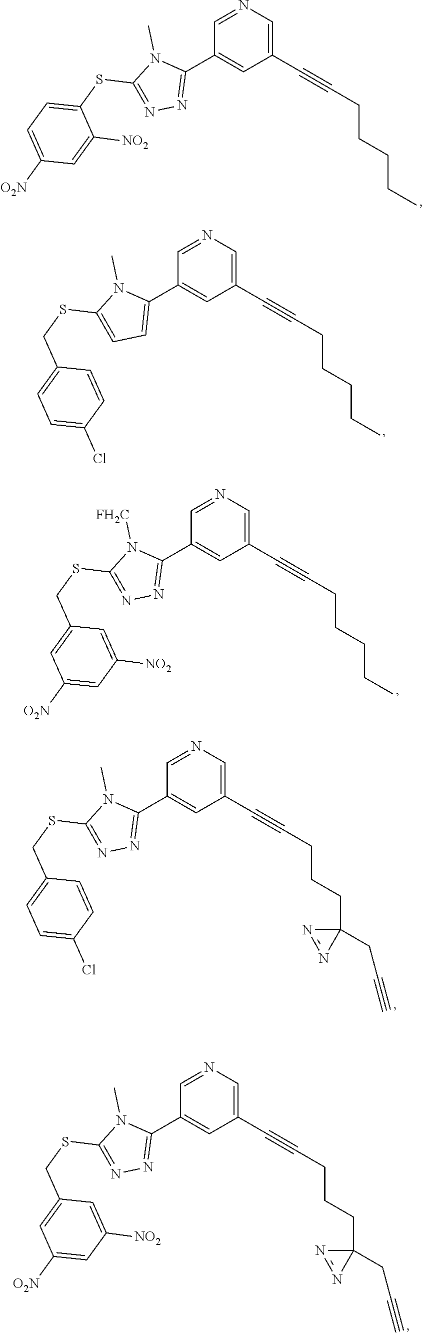

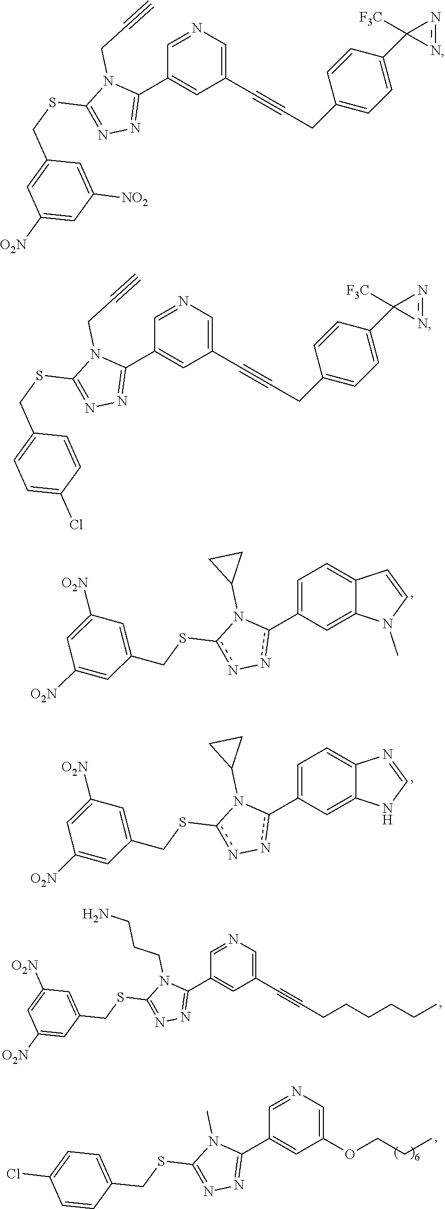

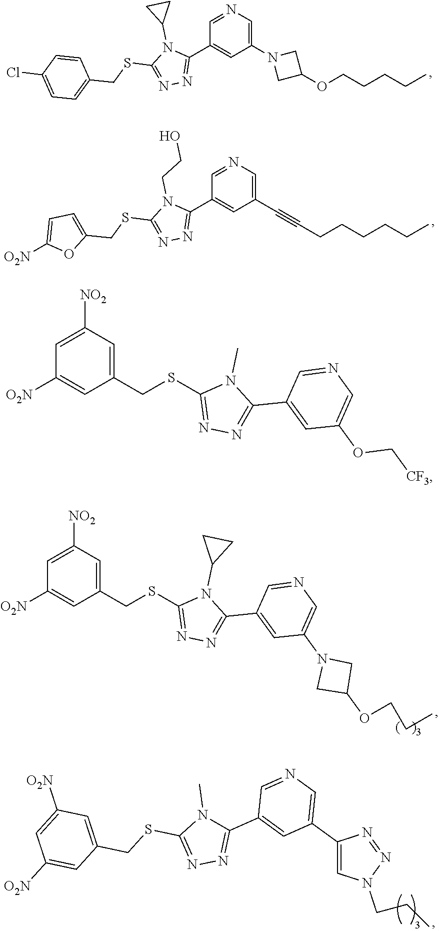

a salt thereof, or any combination thereof.

Aspect 19. A compound of Formula II or a pharmaceutically acceptable salt thereof,

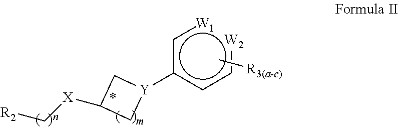

wherein W₁ and W₂ independently comprise N or CR₁₀;
wherein X comprises S, O, NH, NR₄, or CH₂;
wherein Y comprises N, NR₉, or CH;
wherein m and n are independently from 0 to 9; and
wherein each of R₂ and R₉, if present, independently comprises hydrogen, halogen, hydroxyl, cyano, nitro, carboxyl, (carboxyl ester)amino, (carboxyl ester)oxy, acyl, formyl; or substituted or unsubstituted C1-C10 alkyl, C3-C7 cycloalkyl, C3-C7 heterocycloalkyl, alkenyl, alkoxy, alkynyl, amino, aminosulfinyl, sulfinyl, sulfonyl, substituted sulfinyloxy, sulfonyloxy, aminosulfonyloxy, aminosulfinyloxy, aminosulfonylamino, acylamino, aminocarbonyloxy, aminocarbonylamino, aminothiocarbonylamino, aminocarbonyl, aminocarbonyl, acyloxy, C3-C7 aryl, or C3-C7 heteroaryl;
wherein each of R_{3a-3c} independently comprises hydrogen, halogen, hydroxyl, cyano, nitro, carboxyl, (carboxyl ester)amino, (carboxyl ester)oxy, acyl, formyl; or substituted or unsubstituted C1-C10 alkyl, C3-C7 cycloalkyl, C3-C7 heterocycloalkyl, alkenyl, alkoxy, alkynyl, amino, aminosulfinyl, sulfinyl, sulfonyl, substituted sulfinyloxy, sulfonyloxy, aminosulfonyloxy, aminosulfinyloxy, aminosulfonylamino, acylamino, aminocarbonyloxy, aminocarbonylamino, aminothiocarbonylamino, aminocarbonyl, aminocarbonyl, acyloxy, C3-C7 aryl, or C3-C7 heteroaryl, or wherein one of R_{3a-3c} is an N, NR₁₁, CH, or CH₂ group connected to an R₁₀ by a substituted or unsubstituted C1-C3 alkyl chain;
wherein each R₁₀ independently comprises H or is an N, NR₁₂, CH, or CH₂ group connected to one of R_{3a-3c} by a substituted or unsubstituted C1-C3 alkyl chain; and
wherein R₁₁ and R₁₂, if present, independently comprise H or C1-C4 alkyl.

Aspect 20. The compound of aspect 19, wherein W₁ is N, W₂ is CR₁₀, and R₁₀ is H.

Aspect 21. The compound of aspect 19 or 20, where Y is N.

Aspect 22. The compound of any one of aspects 19-21, wherein X is O.

Aspect 23. The compound of any one of aspects 19-21, wherein X is S.

Aspect 24. The compound of any one of aspects 19-23, wherein m is 1.

Aspect 25. The compound of any one of aspects 19-23, wherein m is 2.

Aspect 26. The compound of aspect 25, wherein the compound has a stereochemistry of (R) or (S) at a carbon indicated by *.

Aspect 27. The compound of any one of aspects 19-26, wherein R₂ is

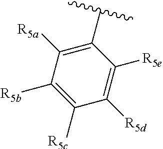

and R₅ₐ-R₅ₑ are independently hydrogen, halogen, cyano, or nitro.

Aspect 28. The compound of aspect 27, wherein R_{5b} and R_{5d} are nitro and R₅ₐ, R_{5c}, and R₅ₑ are hydrogen.

Aspect 29. The compound of aspect 27, wherein R₅ₐ, R_{5b}, R_{5d}, and R₅ₑ, are hydrogen and R_{5c} is chloro.

Aspect 30. The compound of any one of aspects 19-29, wherein n is 0 or 1.

Aspect 31. The compound of any one of aspects 19-30, wherein the compound is

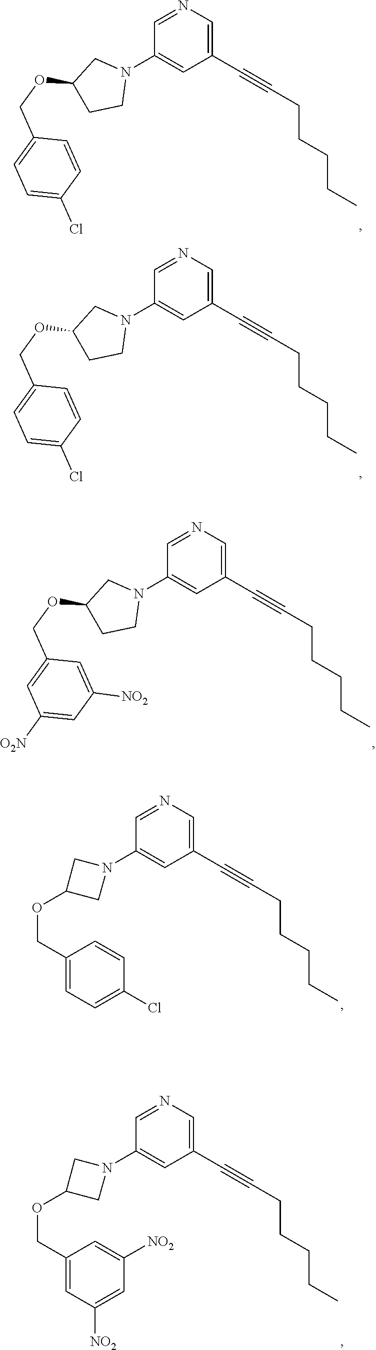 [SMILES: CCCCCC#Cc1cncc(N2CC[C@@H](OCc3ccc(Cl)cc3)C2)c1 ; CCCCCC#Cc1cncc(N2CC[C@H](OCc3ccc(Cl)cc3)C2)c1 ; CCCCCC#Cc1cncc(N2CC[C@@H](OCc3cc([N+](=O)[O-])cc([N+](=O)[O-])c3)C2)c1 ; CCCCCC#Cc1cncc(N2CC(OCc3ccc(Cl)cc3)C2)c1 ; CCCCCC#Cc1cncc(N2CC(OCc3cc([N+](=O)[O-])cc([N+](=O)[O-])c3)C2)c1]

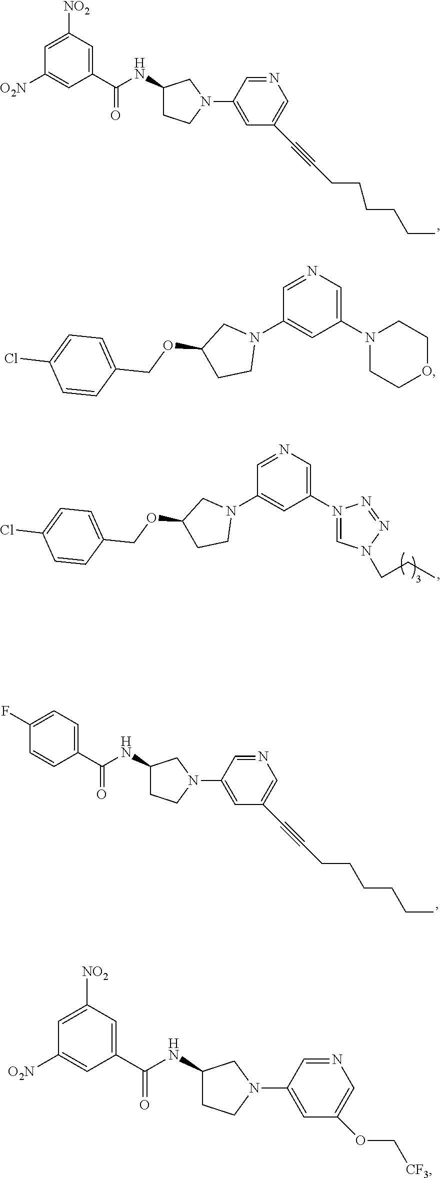

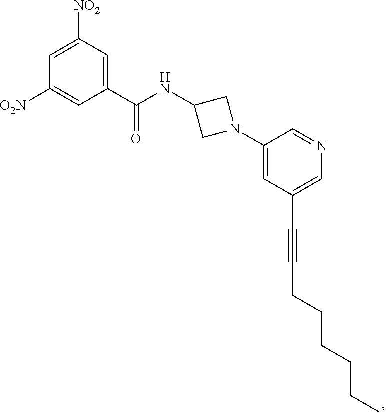 [SMILES: CCCCCCC#Cc1cncc(N2CC(NC(=O)c3cc([N+](=O)[O-])cc([N+](=O)[O-])c3)C2)c1]

a salt thereof, or any combination thereof.

Aspect 32. A compound of Formula III or a pharmaceutically acceptable salt thereof,

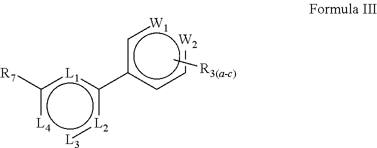

wherein W₁ and W₂ independently comprise N or CH;
wherein each of L₁, L₂, L₃, and L₄ independently comprises N or CH, or, optionally, one of L₁, L₂, L₃, or L₄ independently comprises CR₈;

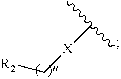

wherein R₇, or, if present, R₈, comprises R²;
wherein X comprises S, O, NH, NR₄, or CH₂;
wherein n is from 0 to 9; and
wherein each of R₂ and R_{3a-3c} comprises hydrogen, halogen, hydroxyl, cyano, nitro, carboxyl, (carboxyl ester)amino, (carboxyl ester)oxy, acyl, formyl; or substituted or unsubstituted C1-C10 alkyl, C3-C7 cycloalkyl, C3-C7 heterocycloalkyl, alkenyl, alkoxy, alkynyl, amino, aminosulfinyl, sulfinyl, sulfonyl, substituted sulfinyloxy, sulfonyloxy, aminosulfonyloxy, aminosulfinyloxy, aminosulfonylamino, acylamino, aminocarbonyloxy, aminocarbonylamino, aminothiocarbonylamino, aminocarbonyl, aminocarbonyl, acyloxy, C3-C7 aryl, or C3-C7 heteroaryl.

Aspect 33. The compound of aspect 32, wherein W₁ is N and W₂ is CH.

Aspect 34. The compound of aspect 32 or 33, wherein R₂ is

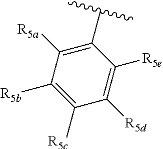

and R₅ₐ-R₅ₑ are independently hydrogen, halogen, cyano, or nitro.

Aspect 35. The compound of aspect 34, wherein R_{5b} and R_{5d} are nitro and R₅ₐ, R_{5c}, and R₅ₑ are hydrogen.

Aspect 36. The compound of aspect 34, wherein R₅ₐ, R_{5b}, R_{5d}, and R₅ₑ, are hydrogen and R_{5c} is chloro.

Aspect 37. The compound of any one of aspects 32-36, wherein n is 0 or 1.

Aspect 38. The compound of any one of aspects 32-37, wherein X is O.

Aspect 39. The compound of any one of aspects 32-37, wherein X is S.

Aspect 40. The compound of any one of aspects 32-39, wherein L₁, L₂, and L₄ are CH and L₃ is N.

Aspect 41. The compound of any one of aspects 32-40, wherein the compound is

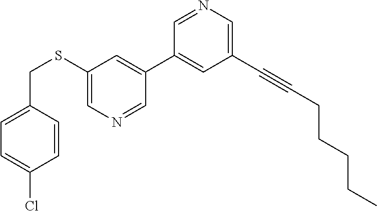 [SMILES: CCCCCC#Cc1cncc(-c2cncc(SCc3ccc(Cl)cc3)c2)c1]

or a salt thereof.

Aspect 42. The compound of any one of aspects 1-41, wherein the salt is a hydrochloride salt.

Aspect 43. The compound of any one of aspects 1-42, wherein the compound has an IC₅₀ of less than 1 μM against Mycobacterium tuberculosis.

Aspect 44. The compound of any one of aspects 1-43, wherein the compound exhibits an MraY inhibition of at least about 45% at a 250 μM concentration.

Aspect 45. The compound of any one of aspects 1-44, wherein the compound is not cytotoxic.

Aspect 46. A pharmaceutical composition comprising the compound or pharmaceutically acceptable salt thereof of any one of aspects 1-45.

Aspect 47. The pharmaceutical composition of aspect 46, further comprising at least one excipient.

Aspect 48. The pharmaceutical composition of aspect 46 or 47, wherein the pharmaceutical composition is administered to a subject orally, by inhalation, parenterally, intravenously, mucosally, or any combination thereof.

Aspect 49. A method for treating a bacterial infection, the method comprising administering to a subject a therapeutically effective amount of the compound of any one of aspects 1-44 or the pharmaceutical composition of any one of aspects 46-48.

Aspect 50. The method of aspect 48, wherein the bacterial infection comprises multidrug-resistant tuberculosis, extensively drug-resistant tuberculosis, or drug-sensitive tuberculosis.

Aspect 51. The method of aspect 49 or 50, wherein the subject is a human.

### EXAMPLES

The following examples are put forth so as to provide those of ordinary skill in the art with a complete disclosure and description of how the compounds, compositions, articles, devices and/or methods claimed herein are made and evaluated, and are intended to be purely exemplary of the disclosure and are not intended to limit the scope of what the inventors regard as their disclosure. Efforts have been made to ensure accuracy with respect to numbers (e.g., amounts, temperature, etc.), but some errors and deviations should be accounted for. Unless indicated otherwise, parts are parts by weight, temperature is in ° C. or is at ambient temperature, and pressure is at or near atmospheric.

### Example 1: General Methods

Hydrazide 1 was synthesized in 2 steps from commercially available methyl-5-bromonicotinates. The hydrazide was refluxed in ethanol with various substituted isothiocyanate to obtain the 1,4-disubstituted thiosemicarbazides which were not isolated. The 1,4-disubstituted thiosemicarbazide was then treated with 10% aqueous NaOH at 60-80° C. for 4-16 h to give the 1,2,4-triazole-3-thiol. Alkylation of the thiols with various arylmethyl halides was undertaken to obtain the final products. Furthermore, taking advantage of the acidic nature of the aromatic thiol, some aromatic substituents of the western were installed by Mitsunobu reaction using arylmethyl alcohol in place of the bromides (3v). To determine the effect of the chain length connecting the sulfur heteroatom to the western ring, arylethyl bromide was reacted with 1, 2, 4-triazole-3-thiols (2) to obtain C2 linker analog; while palladium catalyzed coupling of 4-chlorophenylbromide directly with 2 was used to obtain the CO analog (6c). Nucleophilic aromatic substitution was employed to install various aromatic rings bearing electron withdrawing nitro groups in para or meta to the leaving group.

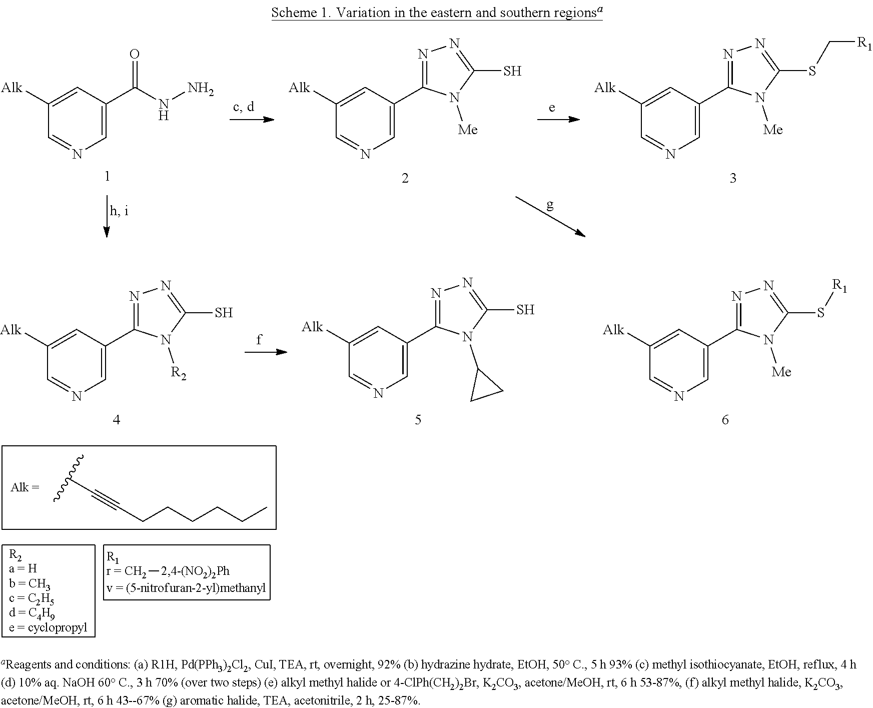

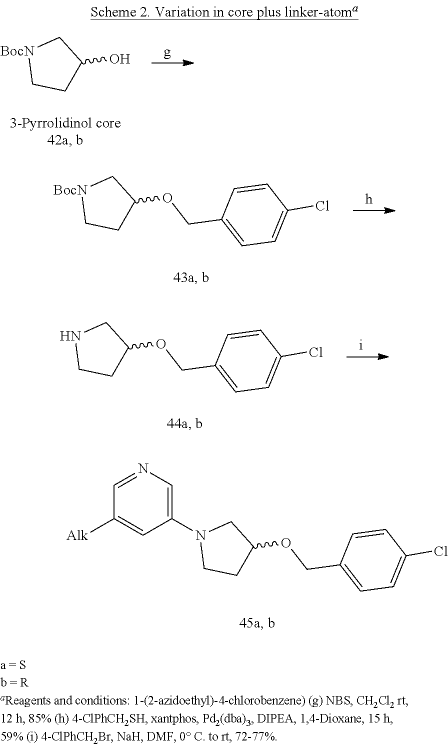

### Example 2: Compounds and Activity

### In Vitro Antimycobacterial Activity

All final compounds were tested for their antimycobacterial activity against Mtb (H37Rv) using the Microplate Alamar Blue Assay (MABA). WT Mtb Erdman or ΔcydA Mtb was cultured in Middlebrook 7H9 liquid media supplemented with 60 μL/L oleic acid, 5 g/L bovine serum albumin, 2 g/L dextrose, 0.003 g/L catalase (OADC), 0.5% glycerol, and 0.05% polysorbate 80 at 37° C. The bacteria were inoculated at a final OD600 of 1.6×10⁻³ in 200 μL per well in 96-well plates with two-fold titrations of compounds. The concentration of DMSO was maintained at 1% for all wells to avoid toxicity. The 96-well plates were incubated in a humidified incubator at 37° C. and 5% CO₂ for one week. After the week incubation, 32.5 μL of a mixture containing an 8:5 ratio of 0.6 mM resazurin (Sigma) dissolved in 1×PBS to 20% polysorbate 80 was added and the production of fluorescent resorufin was measured after incubation at 37° C. in 5% CO₂ for 24 hours. Relative fluorescence units (RFU) were measured using a BioTek Synergy H1 Microplate Reader H1M at an excitation A of 530 nm and an emission λ of 590 nm. Media in the absence of bacteria served as the negative control, and media with bacteria in the absence of compound served as the positive control. Percent inhibition was calculated as follows:

$$( 1 - \frac{sample RFU - negative control RFU}{positive control RFU - negative control RFU} ) \times 100 \%$$

Percent inhibition was input into PRISM GraphPad to calculate IC50 values by plotting as a non-linear regression curve.

The 3,5-dinitrobenzyl analog showed superior antimycobacterial activity compared to the hit with an activity of 0.5 μM. With the concern of toxicity associated with aromatic nitro compounds, replacement of at least one of the nitro with CF₃ or halogen was attempted. This, however, led to a complete loss of activity. Furthermore, three compounds bearing a nitro substituted five membered aromatic group were tested. Of the three, the nitrofuran bearing analog was most potent.

The lack of activity in some compounds suggested that substitution at 4N (substituent R1 (north/south) is important for antimycobacterial activity. The ethyl and cyclopropyl substituted analogs were slightly more active than initial hits based on docking studies, while the n-butyl analog was inactive. Furthermore, the position, however, did not tolerate a phenyl or benzyl group perhaps due to steric hindrances or the increase in lipophilicity with the increased number of carbons.

### SAR Studies

Structure activity relationship (SAR) studies were conducted for some variants of compounds having Formula Ia. Lipophilicity (as c Log P calculated by the SwissADME web service) and IC₅₀ for wild-type (WT) Mtb as determined by the MABA assay described herein (n=1) is presented in Tables 1, 2, and 3.

TABLE 1
SAR for Compounds of Formula Ia

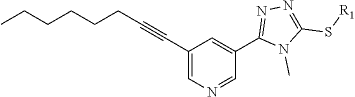

| R¹ | cLogP | IC₅₀ (μM) |
|---|---|---|
| 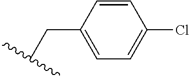 [SMILES: *Cc1ccc(Cl)cc1] | 5.5 | 5.3 |
| 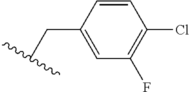 [SMILES: *Cc1ccc(Cl)c(F)c1] | 6.0 | 4.1 |
| 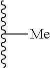 | 3.9 | 48 |
| 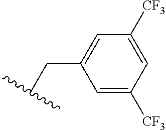 [SMILES: *Cc1cc(C(F)(F)F)cc(C(F)(F)F)c1] | 7.2 | >200 |
| 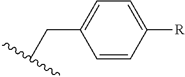 | | |
| R = H | 5.1 | 175 |
| R = Br | 5.6 | 4.6 |
| R = F | 5.4 | 6.9 |
| R = Me | 5.4 | 6.1 |
| R = MeO | 5.1 | 8.7 |
| R = CN | 4.8 | 8.9 |
| R = NO₂ | 4.2 | 5.9 |
| R = CF₃ | 6.1 | 44 |
| R = Ph | 6.3 | 197 |
| 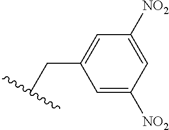 [SMILES: *Cc1cc([N+](=O)[O-])cc([N+](=O)[O-])c1] | 3.5 | 0.34 |
| 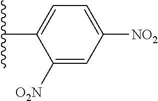 [SMILES: *c1ccc([N+](=O)[O-])cc1[N+](=O)[O-]] | 3.4 | 1.4 |
| 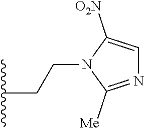 [SMILES: *CCn1c([N+](=O)[O-])cnc1C] | 3.5 | 22 |
| 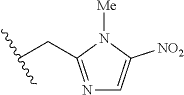 [SMILES: *Cc1ncc([N+](=O)[O-])n1C] | 3.2 | 94 |
| 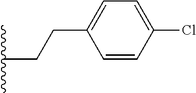 [SMILES: *CCc1ccc(Cl)cc1] | 5.9 | 8.2 |
| 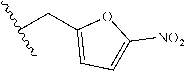 [SMILES: *Cc1ccc([N+](=O)[O-])o1] | 3.9 | 0.55 |
| 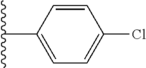 [SMILES: *c1ccc(Cl)cc1] | 5.5 | 13 |
| 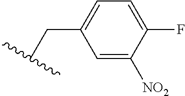 [SMILES: *Cc1ccc(F)c([N+](=O)[O-])c1] | 4.6 | 44 |
| 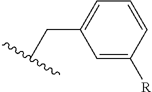 | | |
| R = Cl | 5.6 | 15 |
| R = NO₂ | 4.3 | 31 |
| 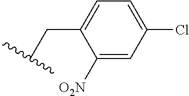 [SMILES: *Cc1ccc(Cl)cc1[N+](=O)[O-]] | 4.9 | >200 |
| 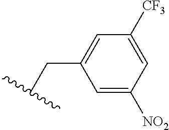 [SMILES: *Cc1cc([N+](=O)[O-])cc(C(F)(F)F)c1] | 5.4 | >200 |
| 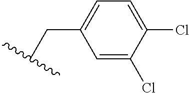 [SMILES: *Cc1ccc(Cl)c(Cl)c1] | 6.2 | 6.8 |
| 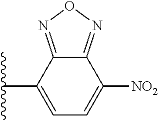 [SMILES: *c1ccc([N+](=O)[O-])c2nonc12] | 3.7 | 27 |
| 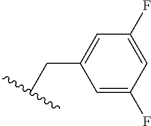 [SMILES: *Cc1cc(F)cc(F)c1] | 5.6 | 8.0 |
| 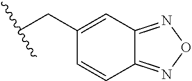 [SMILES: *Cc1ccc2nonc2c1] | 4.5 | >200 |

TABLE 2
SAR for Compounds of Formula la

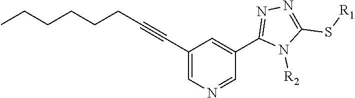

| R¹ | R² | cLogP | IC₅₀ (μM) |
|---|---|---|---|
| 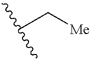 [SMILES: *CC] | 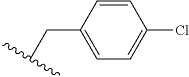 [SMILES: *Cc1ccc(Cl)cc1] | 5.9 | 4.7 |
| 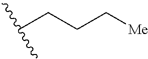 [SMILES: *CCCC] | | 6.6 | >200 |
| 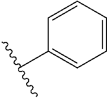 [SMILES: *c1ccccc1] | | 6.8 | >200 |
| 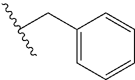 [SMILES: *Cc1ccccc1] | | 6.8 | >200 |
| 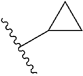 [SMILES: *C1CC1] | | 6.1 | 3.3 |
| 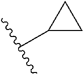 [SMILES: *C1CC1] | 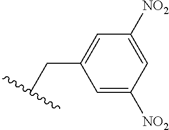 [SMILES: *Cc1cc([N+](=O)[O-])cc([N+](=O)[O-])c1] | 4.1 | 1.1 |
| 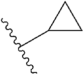 [SMILES: *C1CC1] | 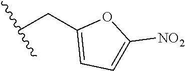 [SMILES: *Cc1ccc([N+](=O)[O-])o1] | 4.3 | <0.78 |

TABLE 3
SAR for Compounds of Formula la

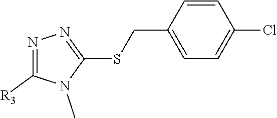

| R³ | cLogP | IC₅₀ (μM) |
|---|---|---|
| 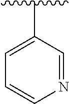 [SMILES: *c1cccnc1] | 3.1 | >200 |
| 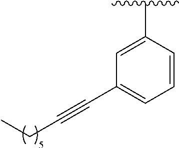 | 6.4 | 8.5 |
| 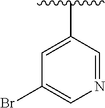 [SMILES: *c1cncc(Br)c1] | 3.7 | >200 |
| 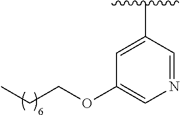 | 5.4 | 3.5 |
| 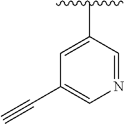 [SMILES: *c1cncc(C#C)c1] | 3.5 | 86 |
| 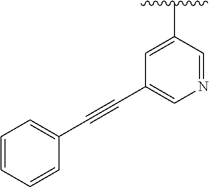 [SMILES: *c1cncc(C#Cc2ccccc2)c1] | 4.7 | 4.0 |
| 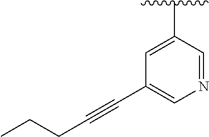 [SMILES: *c1cncc(C#CCCC)c1] | 4.5 | 15 |
| 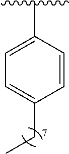 | 6.6 | >200 |
| 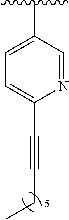 | 4.6 | >200 |

### MraY Docking Studies

Since the hit was obtained from virtual screening for MraY inhibitors the hit compound and the most active compounds were docked into the crystal structure of MraYMtb. A homology model of MraYMtb was recently reported, which was constructed using MraY from Aquifex aeolicus (PDB ID: 5CKR) a template. The docking grid box (10×10×10 Å) was defined by picking the muraymycin D1 (MD1) the co-crystalized ligand in the homology model, using the OPLS3e force field default parameters.

### MraY Enzymatic Assay

Since the active site is well-conserved, membranes containing overexpressed MraY from E. coli, B. subtilis, P. aeruginosa, and S. aureus were used. The membrane fractions from native strains from M. smegmatis and Mtb have extremely low levels of MraY activity, which would be hard to detect and assay the inhibitors, therefore only the active compounds against the recombinant clones were advanced to the Msmeg and Mtb membrane fractions. The range of enzymes allowed us to assess the selectivity of the designed inhibitors and VS hits. UDP-MurNAc-L-Ala-D-Glu-L-Lys(Ne-dansyl)-D-Ala-D-Ala was prepared by following a published procedure. The MraY-catalyzed reaction was monitored on an LS55 fluorimeter (lex 340 nm, lex 530 nm; PerkinElmer). The formation of dansyl-lipid I, membrane-bound E. coli MraY (15 mL of 0.6 mg mL-1 stock) was monitored by incubating UDP-MurNAc-L-Ala-D-Glu-L-Lys(N-dansyl)-D-Ala-D-Ala (17.5 mm), lipid carrier undecaprenyl phosphate (39 mm) or heptaprenyl phosphate (59 mm), with MgCl₂ (20 mm) in Tris buffer (83 mm, pH 7.5), in a total volume of 0.5 mL.

Compounds were immediately moved into Mtb to prioritize structures that retain activity in Mtb. If a compound shows activity against Mtb in any of three assays, it was considered active in Mtb. In addition to testing the compounds against the drug sensitive Mtb Erdman and HN878 strains, their efficacy was also examined against MDR-TB clinical isolates from BEI Resources. Selected results for testing against Erdman and HN878 Mtb strains are presented in Table 4:

TABLE 4
Efficacy against Mtb Strains
IC₅₀ ± S.D. (μM)^{[a]}

| compd. | Erdman | HN878 |
|---|---|---|
| 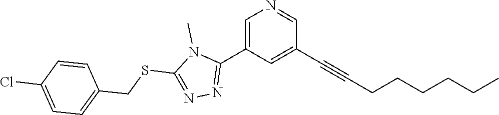 [SMILES: CCCCCCC#Cc1cncc(-c2nnc(SCc3ccc(Cl)cc3)n2C)c1] | 1.5 ± 0.19 | 0.85 ± 0.043 |
| 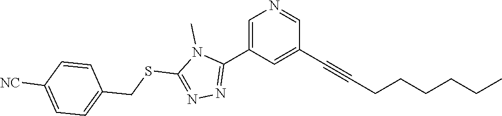 [SMILES: CCCCCCC#Cc1cncc(-c2nnc(SCc3ccc(C#N)cc3)n2C)c1] | 5.3 ± 1.17 | 2.3 ± 0.2 |
| 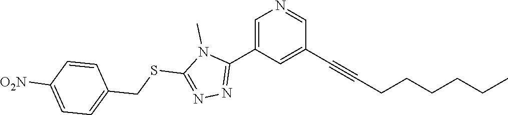 [SMILES: CCCCCCC#Cc1cncc(-c2nnc(SCc3ccc([N+](=O)[O-])cc3)n2C)c1] | 2.9 ± 0.45 | 1.3 ± 0.15 |
| 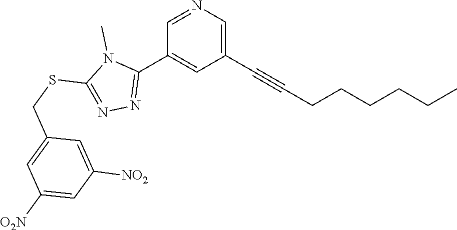 [SMILES: CCCCCCC#Cc1cncc(-c2nnc(SCc3cc([N+](=O)[O-])cc([N+](=O)[O-])c3)n2C)c1] | 0.17 ± 0.016 | 0.061 ± 0.012 |
| INH^{[b]} | 0.18 ± 0.014 | 0.12 ± 0.00051 |

^{[a]}Anti-Mtb activity in wild-type (WT) Mtb Erdman and HN878 strains were determined using the MABA described herein (n = 3).
^{[b]}INH = isoniazid.

As an alternative assay for compounds that interfere with the MABA assay, zone-of-inhibition assays were performed. For these assays, a lawn of Mtb is plated on standard 7H10 agar media and a disk spotted with the compound is placed in the center of the lawn. After incubation at 37° C. in 5% CO₂, the zone of clearing was measured around the disks spotted with compound. DMSO has no effect on Mtb growth in this assay and does not generate a zone of clearing on its own.

In a third assay, whether the compounds are bactericidal or bacteriostatic was determined by treating with a range of concentrations in liquid cultures and plating the surviving bacteria to calculate CFUs remaining at different time points over the course of 3 weeks.

### Inhibition of Mtb Infection in Macrophages

During infection, Mtb survives and replicates within multiple cell types, including macrophages. New antimycobacterials need to be effective against intracellular bacteria to successfully treat the infection. To ascertain if compounds can target intracellular Mtb, murine and human macrophages were infected in culture in the presence or absence of compounds and Mtb survival was monitored by plating serial dilutions to enumerate CFUs at 4, 24, 48, 72, and 96 hours post-infection. By testing a range of compound concentrations, the IC₉₀ for inhibition of Mtb survival in macrophages was determined. To monitor toxicity of compounds to the macrophages, host cell viability was compared by trypan blue dye exclusion assays in conditions with or without Mtb infection and untreated or compound-treated.

### Zone of Inhibition Assays for Antimicrobial Activity

Zone of inhibition assays for antimicrobial activity. Bauer-Kirby disc diffusion assays were used against a panel of bacteria containing methicillin-resistant S. aureus, E. coli, P. aeruginosa, K. pneumoniae, and E. faecium VRE. Briefly, the bacteria were plated on agar media and inhibitors dissolved in DMSO were spotted on a disk in the center of a lawn of bacteria. After incubation at 37° C., the radius of the zone absent of bacterial growth was measured. DMSO was used as a control and should not have an effect on bacterial growth and survival in this assay.

### Example 3: Synthesis and Characterization

All solvents and reagent were purchase from standard commercial vendors and used without further purification. Synthetic reactions were monitored using thin layer chromatography (TLC) (Sorbtech silica XG TLC plates) and visualized under UV at 254 nm or with appropriate staining. Purification was undertaking using Flash column chromatography on Biotage Isolera One with Biotage SNAP 10 g-50 g cartridges. NMR spectra were recorded on a Bruker Avance-500 (500 MHz) or Bruker Avance-400 (400 MHz) spectrometer at 298.15 K. Chemical shifts are reported in ppm using deuterated solvents and residual solvent signal or TMS as reference. Data were reported as: s=singlet, sbr=broad singlet, d=doublet, t=triplet, q=quartet, p=pentet, m=multiplet, b=broad, ap=apparent; coupling constants, J, in Hz. For high-resolution mass spectrometry (HRMS), quadruple-TOF was used to obtain the data both in positive and negative modes. Attenuated total reflectance infra-red (ATR-IR) was taken using an Agilent Technologies Cary 600 series FTIR Spectrometer. Purity (95%) was determined by LC-MS analysis.

General Procedure for Sonogashira Coupling of Bromide with Corresponding Alkyne

A mixture of the appropriate aryl bromide (1 Eq), copper(I) iodide (0.1 Eq), and bis(triphenylphosphine)palladium (II) chloride (0.05 Eq) in DMF was purged with argon for 10 min and treated with the corresponding alkyne (1 Eq) previously stirred with triethylamine (1.9 g, 2 Eq) under argon. The resultant mixture was stirred at rt 16-24 h. After completion of reaction as determined by TLC, the reaction mixture was diluted with NaHCO₃ and extracted with diethyl ether (×2), washed with water and brine, dried with Na₂SO₄, and purified with flash chromatography to give final product.

### General Procedure for Synthesis of Acyl Hydrazide

To the methyl ester of the appropriate carboxylic acid (1 Eq) dissolved in MeOH (30 mL) was added 64% solution hydrazine hydrate (2 Eq) gradually and reflux for 5-16 hour. On completion of reaction as determined by TLC, the reaction mixture was brought to room temperature and the precipitated product was filtered and rinsed with methanol and n-hexane to obtain white crystalline solid. Product was used for the next step without further purification.

### General Procedure for Synthesis of 4,5-substituted-1,2,4-triazole-2-thiol

Acyl hydrazide (1 Eq) and corresponding isothiocyanate (1 Eq) dissolved in absolute ethanol (30 mL) was refluxed for 4 h. After completion of reaction as confirmed by TLC, the solvent was removed under reduced pressure to obtain the corresponding 1,4-substituted thiosemicarbazides. To the residue was then added 15 mL of 10% NaOH and the mixture was stirred for 3 h at 60° C. On completion of the reaction, reaction mixture was brought to rt and acidified with HCl to a pH of 5-6. The precipitate formed was then filtered and rinsed with a little water to give the corresponding 4,5-substituted-1,2,4-triazole-2-thiol and except if stated otherwise, was used without further purification.

### General Procedures for Alkylation of 4,5-substituted-1,2,4-triazole-2-thiol

Method 1: To the appropriate alkylating agent (bromide or chloride) (1.2 Eq) and corresponding 1,2,4-triazole-3-thiol (1 Eq) dissolved in MeOH/Acetone (1:2) was added K₂CO₃ (2 Eq) and the mixture was stirred for 4-12 h at room temperature. On the completion of the reaction as determined by TLC, the solvent was evaporated under reduced pressure. EtOAc (30 mL) was added to the residue, which was then washed with water and with brine and then dried over Na₂SO₄, concentrated under low pressure and the residue was purified by column chromatography (EtOAc/hexane).

Method 2: The appropriate alkylating agent (1.2 Eq) was added to a solution of the corresponding 1,2,4-triazole-3-thiol (1 Eq) and TEA (2 Eq) in acetonitrile and stirred for 4 h at room temperature. The residue was then dissolved in EtOAc (30 mL) and washed with 5% Na₂CO₃, brine and dried over Na₂CO₃, concentrated under low pressure and the residue was purified by column chromatography (EtOAc/Hexane).

Method 3: To appropriate arylmethyl alcohol (1.2 Eq) dissolved in anhydrous THF (2 mL) was added triphenylphosphine (1.2 Eq) and 1,2,4-triazole-3-thiol (1 Eq) at 0° C. under argon. After stirring for 30 min DIAD (1 Eq) was added dropwise. The mixture was then warmed to room temperature and stirred for 3 h. Solvent was removed in vacuo, and the residue was purified by flash column chromatography (2-5% MeOH/CH₂Cl₂) to afford the final product.

### 3-(4-methyl-5-(((5-nitrofuran-2-yl)methyl)thio)-4H-1,2,4-triazol-3-yl)-5-(oct-1-yn-1-yl)pyridine (TiB-02-212)

2-(bromomethyl)-5-nitrofuran (53 mg, 1.2 Eq, 0.26 mmol) was reacted with 4-methyl-5-(5-(oct-1-yn-1-yl)pyridin-3-yl)-4H-1,2,4-triazole-3-thiol (64 mg, 1 Eq, 0.21 mmol) following method 2. Final product was purified by flash chromatography (mobile phase: 5% MeOH/CH₂Cl₂). Yield 54% as brown semisolid; ¹H NMR (400 MHz, chloroform-d) δ 8.73 (dd, J=7.3, 2.0 Hz, 2H), 7.95 (t, J=2.1 Hz, 1H), 7.22 (d, J=3.7 Hz, 1H), 6.68 (d, J=3.7 Hz, 1H), 4.58 (s, 2H), 3.60 (s, 3H), 2.44 (t, J=7.1 Hz, 2H), 1.62 (p, J=7.1 Hz, 2H), 1.51-1.40 (m, 2H), 1.32 (tt, J=7.4, 3.3 Hz, 4H), 0.97-0.83 (m, 3H). ¹³C NMR (101 MHz, CDCl₃) δ 153.87, 153.66, 153.48, 151.00, 146.84, 138.18, 122.75, 121.85, 113.01, 112.75, 96.26, 76.53, 31.88, 31.43, 29.24, 28.72, 28.50, 22.67, 19.61, 14.19. HRMS (ESI): m/z [M+H]⁺ Calcd for [C₂₁H₂₃N₅O₃S+H]⁺ 426.1600, found 436.1601.

### Example 4: Cytotoxicity Assays

Cytotoxicity assays were conducted to measure toxicity of compounds at single concentration in Vero TMPRSS2 cells by using quantitation of ATP to indicate metabolically active cells. Vero cells (derived from African Green Monkey kidney epithelial tissue) were grown in Dulbecco's Modified Eagle Medium (DMEM) supplemented with 10% fetal bovine serum (FBS), 10 mM HEPES buffered saline, and 2 mM L-glutamine. 5,000 cells per well were seeded into a 96-well plate containing two-fold titrations of compounds. The concentration of DMSO was maintained at 0.5% for all wells to avoid toxicity. The plates were incubated for 72 hours, at which point they were acclimatized to room temperature for 30 minutes. 25 μL of the substrate/buffer solution from the Promega CellTiter-Glo Luminescent Cell Viability Assay kit was added to each well. The plates were shaken at a low orbital speed for 3 minutes and let rest at room temperature for 10 minutes, after which luminescence was measured using a BioTek Synergy H1 Microplate Reader H1M. The positive control was defined as wells containing cells without compounds.

Percent viability was calculated as follows:

$$( \frac{sample luminescence}{positive control luminescence} ) \times 100 \%$$

Percent viability was input into PRISM GraphPad to calculate CC₅₀ values by plotting as a non-linear regression curve. Results are presented in Table 5 below:

TABLE 5
Cytotoxicity Assays

| Concentration | | % Cell Viability | | |
|---|---|---|---|---|
| (uM) | Compound | Replicate 1 | Replicate 2 | Average |
| 200 mg/mL | Streptomycin | 112.83 | 109.14 | 110.98 |
| 200 mg/mL | Streptomycin | 110.01 | 103.70 | 106.86 |
| 200 mg/mL | Streptomycin | 108.58 | 102.37 | 105.48 |
| 50 | Capuramycin B | 96.50 | 92.96 | 94.73 |
| 50 | remdesivir | 94.32 | 94.19 | 94.25 |
| 50 | remdesivir | 91.48 | 95.46 | 93.47 |
| 50 | Muraymycin D1 | 93.83 | 90.92 | 92.38 |
| 50 | Capuramycin B | 90.49 | 93.64 | 92.07 |
| 50 | Capuramycin B | 90.38 | 92.73 | 91.55 |
| 50 | Muraymycin D1 | 94.27 | 88.18 | 91.22 |
| 50 | Muraymycin D1 | 87.87 | 91.39 | 89.63 |
| 50 | remdesivir | 89.25 | 89.19 | 89.22 |
| 100 | 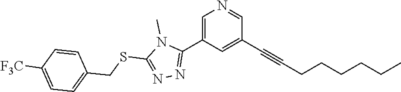 [SMILES: CCCCCCC#Cc1cncc(-c2nnc(SCc3ccc(C(F)(F)F)cc3)n2C)c1] | 74.34 | 66.36 | 70.35 |
| 125 | 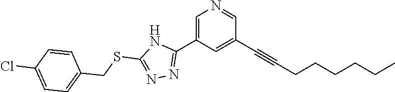 [SMILES: CCCCCCC#Cc1cncc(-c2nnc(SCc3ccc(Cl)cc3)[nH]2)c1] | 68.39 | 67.58 | 67.98 |
| 100 | 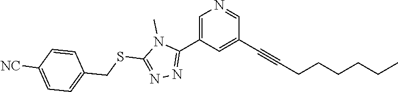 [SMILES: CCCCCCC#Cc1cncc(-c2nnc(SCc3ccc(C#N)cc3)n2C)c1] | 75.34 | 57.31 | 66.33 |
| 100 | 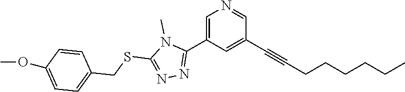 [SMILES: CCCCCCC#Cc1cncc(-c2nnc(SCc3ccc(OC)cc3)n2C)c1] | 60.30 | 48.95 | 54.63 |
| 100 | 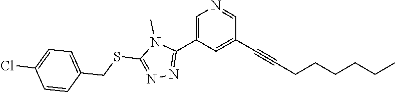 [SMILES: CCCCCCC#Cc1cncc(-c2nnc(SCc3ccc(Cl)cc3)n2C)c1] | 53.54 | 54.56 | 54.05 |
| 125 | 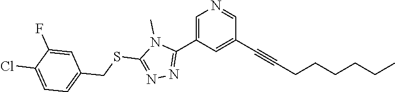 [SMILES: CCCCCCC#Cc1cncc(-c2nnc(SCc3ccc(Cl)c(F)c3)n2C)c1] | 49.91 | 51.65 | 50.78 |
| 100 | CB81 | 45.92 | 42.36 | 44.14 |
| 100 | CB81 | 47.39 | 38.44 | 42.92 |
| 100 | 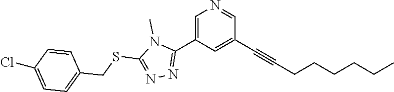 [SMILES: CCCCCCC#Cc1cncc(-c2nnc(SCc3ccc(Cl)cc3)n2C)c1] | 47.04 | 38.53 | 42.78 |
| 100 | 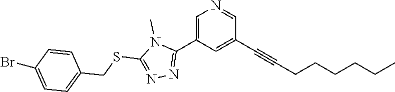 [SMILES: CCCCCCC#Cc1cncc(-c2nnc(SCc3ccc(Br)cc3)n2C)c1] | 42.94 | 41.40 | 42.17 |
| 125 | 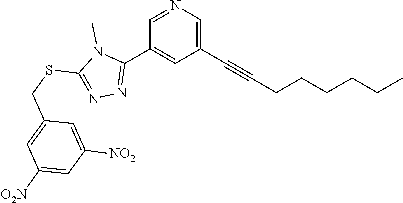 [SMILES: CCCCCCC#Cc1cncc(-c2nnc(SCc3cc([N+](=O)[O-])cc([N+](=O)[O-])c3)n2C)c1] | 42.56 | 40.84 | 41.70 |
| 100 | CB81 | 41.44 | 40.62 | 41.03 |
| 125 | 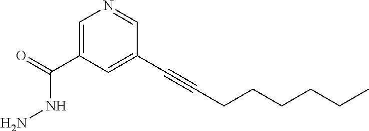 [SMILES: CCCCCCC#Cc1cncc(C(=O)NN)c1] | 34.62 | 38.69 | 36.66 |
| 100 | 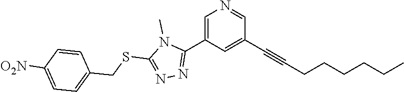 [SMILES: CCCCCCC#Cc1cncc(-c2nnc(SCc3ccc([N+](=O)[O-])cc3)n2C)c1] | 39.61 | 29.56 | 34.59 |
| 100 | 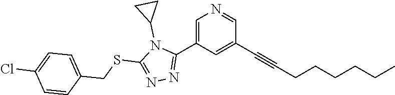 [SMILES: CCCCCCC#Cc1cncc(-c2nnc(SCc3ccc(Cl)cc3)n2C2CC2)c1] | 32.40 | 34.99 | 33.69 |
| 100 | 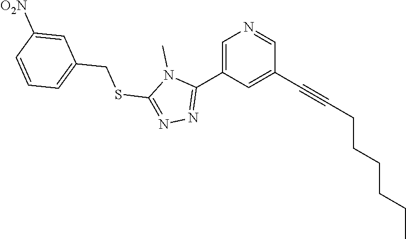 [SMILES: CCCCCCC#Cc1cncc(-c2nnc(SCc3cccc([N+](=O)[O-])c3)n2C)c1] | 26.28 | 26.66 | 26.47 |
| 100 | 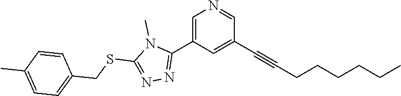 [SMILES: CCCCCCC#Cc1cncc(-c2nnc(SCc3ccc(C)cc3)n2C)c1] | 20.58 | 20.35 | 20.46 |
| 100 | 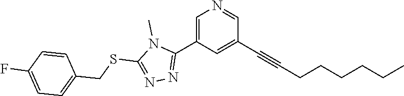 [SMILES: CCCCCCC#Cc1cncc(-c2nnc(SCc3ccc(F)cc3)n2C)c1] | 32.71 | 6.50 | 19.61 |
| 100 | 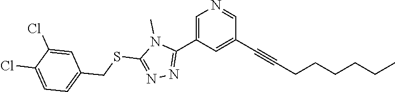 [SMILES: CCCCCCC#Cc1cncc(-c2nnc(SCc3ccc(Cl)c(Cl)c3)n2C)c1] | 19.98 | 17.98 | 18.98 |
| 100 | 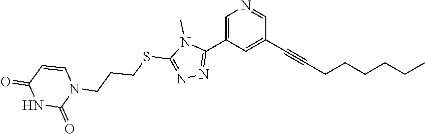 [SMILES: CCCCCCC#Cc1cncc(-c2nnc(SCCCn3ccc(=O)[nH]c3=O)n2C)c1] | 9.07 | 19.21 | 14.14 |
| 100 | 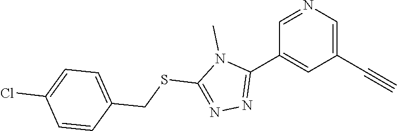 [SMILES: C#Cc1cncc(-c2nnc(SCc3ccc(Cl)cc3)n2C)c1] | 3.02 | 3.18 | 3.10 |
| 100 | 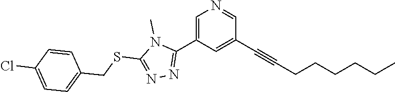 [SMILES: CCCCCCC#Cc1cncc(-c2nnc(SCc3ccc(Cl)cc3)n2C)c1] | 2.25 | 2.47 | 2.36 |
| 100 | 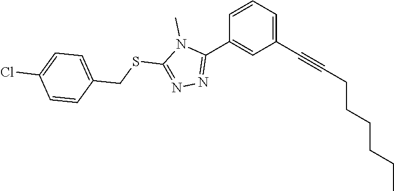 [SMILES: CCCCCCC#Cc1cccc(-c2nnc(SCc3ccc(Cl)cc3)n2C)c1] | 1.97 | 1.58 | 1.77 |
| 100 | 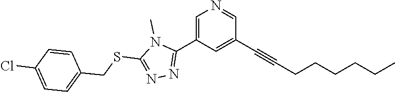 [SMILES: CCCCCCC#Cc1cncc(-c2nnc(SCc3ccc(Cl)cc3)n2C)c1] | 1.57 | 1.76 | 1.67 |
| 100 | 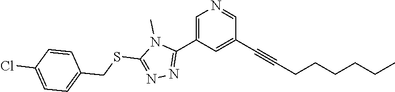 [SMILES: CCCCCCC#Cc1cncc(-c2nnc(SCc3ccc(Cl)cc3)n2C)c1] | 1.41 | 1.70 | 1.56 |
| 125 | 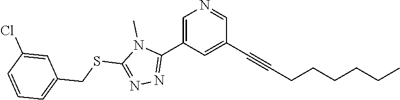 [SMILES: CCCCCCC#Cc1cncc(-c2nnc(SCc3cccc(Cl)c3)n2C)c1] | 2.13 | 0.94 | 1.53 |
| 125 | 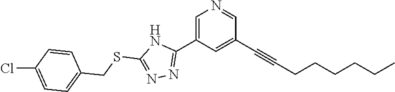 [SMILES: CCCCCCC#Cc1cncc(-c2nnc(SCc3ccc(Cl)cc3)[nH]2)c1] | 1.43 | 1.38 | 1.41 |
| 100 | 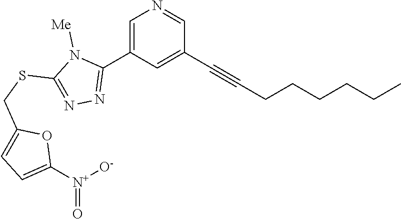 [SMILES: CCCCCCC#Cc1cncc(-c2nnc(SCc3ccc([N+](=O)[O-])o3)n2C)c1] | 1.30 | 1.31 | 1.31 |
| 100 | 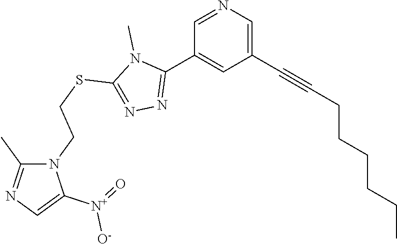 [SMILES: CCCCCCC#Cc1cncc(-c2nnc(SCCn3c([N+](=O)[O-])cnc3C)n2C)c1] | 1.17 | 1.20 | 1.19 |
| 100 | 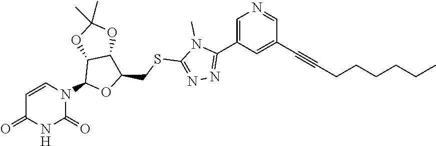 [SMILES: CCCCCCC#Cc1cncc(-c2nnc(SC[C@H]3O[C@@H](n4ccc(=O)[nH]c4=O)[C@@H]4OC(C)(C)O[C@@H]43)n2C)c1] | 1.02 | 1.19 | 1.10 |
| 125 | 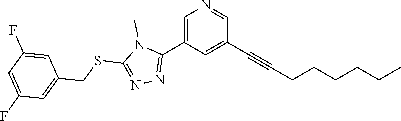 [SMILES: CCCCCCC#Cc1cncc(-c2nnc(SCc3cc(F)cc(F)c3)n2C)c1] | 0.98 | 0.94 | 0.96 |
| 100 | 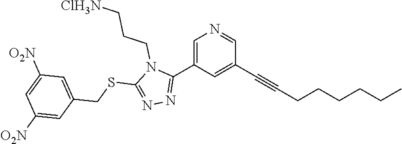 | 0.90 | 0.99 | 0.95 |
| 125 | 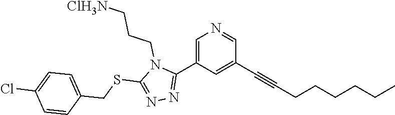 | 0.94 | 0.93 | 0.94 |
| 125 | 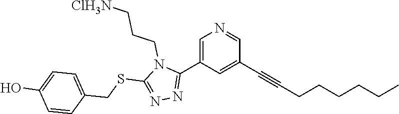 | 0.89 | 0.94 | 0.91 |
| 100 | 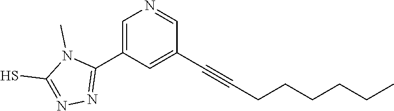 [SMILES: CCCCCCC#Cc1cncc(-c2nnc(S)n2C)c1] | 0.70 | 0.82 | 0.76 |
| 125 | 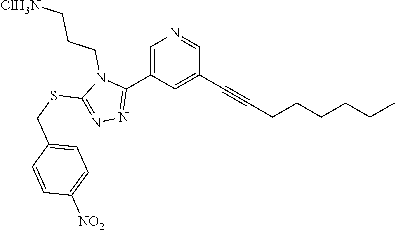 | 0.73 | 0.73 | 0.73 |

Given the toxicity of an initial hit compound in mammalian cells and the potential concern for toxicity more generally with nitro-containing drugs, the selected compounds were also examined for toxicity in a mammalian cell line using Promega's Cell Titer-Glo assay to assess cellular ATP levels as a read-out of viability. The concentration of the selected compound that resulted in a 50% decrease in cellular ATP (CC₅₀) in Vero cells, an African green monkey kidney epithelial cell line, over a 72-hour incubation, was determined. A selectivity index (SI) was then calculated for each compound by determining the ratio of the CC₅₀ in Vero cells to the MABA IC₅₀ in Mtb, where the larger the SI, the safer the compound is in eukaryotic cells relative to its effective dose. From this analysis, multiple highly potent anti-Mtb compounds with favorable selectivity indexes were identified, some with SI >100. Therefore, not only were the SAR efforts successful in identifying compounds with increased potency against Mtb, but also, the toxicity concerns of the original hit compound were overcome. Results for selected compounds are presented in Table 6:

TABLE 6
Cytotoxicity Data for Selected Compounds

| | | IC₅₀ S.D. | | |
|---|---|---|---|---|
| | | (μM) Mtb | CC₅₀ (μM), | selectivity |
| compd. | cLogP^{[a]} | Erdman strain^{[b]} | Vero cells^{[c]} | index (SI) |
| 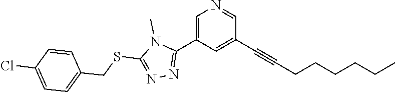 [SMILES: CCCCCCC#Cc1cncc(-c2nnc(SCc3ccc(Cl)cc3)n2C)c1] | 5.5 | 5.4 ± 0.5 | 50 ± 5.5 | 9.3 |
| 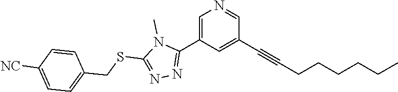 [SMILES: CCCCCCC#Cc1cncc(-c2nnc(SCc3ccc(C#N)cc3)n2C)c1] | 4.8 | 27 ± 0.4 | >100 | >3.7 |
| 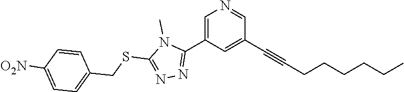 [SMILES: CCCCCCC#Cc1cncc(-c2nnc(SCc3ccc([N+](=O)[O-])cc3)n2C)c1] | 4.2 | 22 ± 13 | 27 ± 5.4 | 1.3 |
| 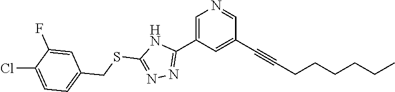 [SMILES: CCCCCCC#Cc1cncc(-c2nnc(SCc3ccc(Cl)c(F)c3)[nH]2)c1] | 6.0 | 5.4 ± 0.03 | >125 | >23 |
| 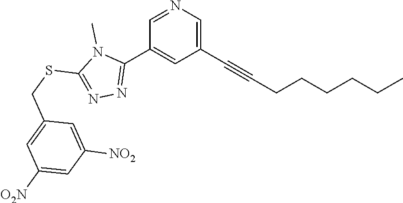 [SMILES: CCCCCCC#Cc1cncc(-c2nnc(SCc3cc([N+](=O)[O-])cc([N+](=O)[O-])c3)n2C)c1] | 3.5 | 0.34 ± 0.01 | >100 | >294 |
| 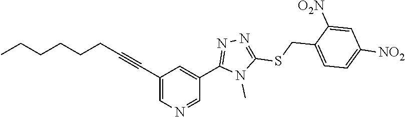 [SMILES: CCCCCCC#Cc1cncc(-c2nnc(SCc3ccc([N+](=O)[O-])cc3[N+](=O)[O-])n2C)c1] | 3.4 | 1.1 ± 0.1 | 28 ± 1.0 | 27 |
| 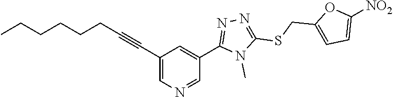 [SMILES: CCCCCCC#Cc1cncc(-c2nnc(SCc3ccc([N+](=O)[O-])o3)n2C)c1] | 3.9 | 0.1 ± 0.01 | 33 ± 2.9 | 314 |
| 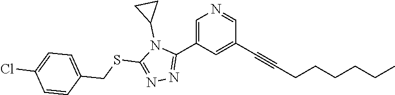 [SMILES: CCCCCCC#Cc1cncc(-c2nnc(SCc3ccc(Cl)cc3)n2C2CC2)c1] | 6.0 | 5.1 ± 0.7 | 25 ± 2.5 | 4.8 |
| 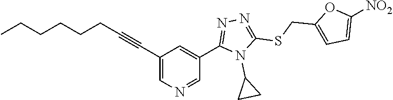 [SMILES: CCCCCCC#Cc1cncc(-c2nnc(SCc3ccc([N+](=O)[O-])o3)n2C2CC2)c1] | 4.2 | 0.05 ± 0.003 | 7.8 ± 0.48 | 165 |
| 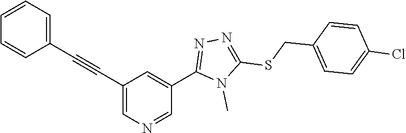 [SMILES: Cn1c(SCc2ccc(Cl)cc2)nnc1-c1cncc(C#Cc2ccccc2)c1] | 4.8 | 2.2 ± 0.4 | >100 | >46 |
| 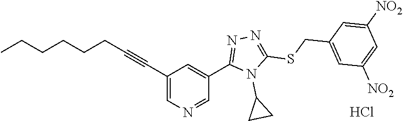 [SMILES: CCCCCCC#Cc1cncc(-c2nnc(SCc3cc([N+](=O)[O-])cc([N+](=O)[O-])c3)n2C2CC2)c1 ; Cl] | 3.7 | 0.95 ± 0.04 | ND | — |

^{[a]}The calculated LogP values were determined using SwissADME web service.
^{[b]}Anti-Mtb activity in wild-type (WT) Mtb Erdman strain was determined using the MABA (n = 3).
^{[c]}CC₅₀ in Vero cells is based on the Promega CellTiter-Glo Luminescent Cell Viability Assay (n = 3).

### Example 5: In Vitro Pharmacokinetic Properties

Having identified nanomolar growth inhibitors of Mtb with good selectivity indices, the aqueous solubility, human plasma protein binding, plasma stability, and liver microsomal stability of selected compounds were determined to establish a baseline for future analogs and provide insight into the potential for future in vivo studies. Certain compounds showed a poor solubility profile comparable to that of reserpine, which is a reference for low solubility (Table 7). It was next investigated whether pyridinium salts improved solubility; however, the improvement was only marginal. The pyridinium salt retained its anti-Mtb potency with an IC₅₀ of 0.95 μM in MABA. It is hypothesized that the poor solubility might be due to the lipophilic side chain in the western region. Lipophilicity in TB drugs is often associated with low solubility that can be addressed by formulation or mode of administration, such as inhalation. Poor Caco-2 cell permeability was also observed, which might be due to the low aqueous solubility and high lipophilicity of these compounds. Solubilizers, such as propylene glycol (PG), hydroxypropyl-beta-cyclodextrin (HP-beta-CD), and nanoparticles, are often used to enhance the bioavailability of drugs with poor aqueous solubility. In terms of metabolic stability, at least one compound exhibited an in vitro intrinsic clearance (CLᵢₙₜ) of 287 and 357 μL/min/mg and degradation half-life (t_{1/2}) of 4.8 and 3.9 min in mouse and human liver microsomes, respectively (Table 7). The presence of the thioether and N-demethylation was identified at position N4- of the 1,2,4-triazole core as potential sources of metabolic instability. However, the introduction of the N4-cyclopropyl group did not circumvent the metabolic instability. Several compounds displayed excellent stability in both mouse and human plasma with t_{1/2}>480 min (Table 7) when compared to propantheline (t_{1/2 human}=21 min; t_{1/2 mouse} 24 min) and warfarin (t_{1/2 human and mouse}>480 min). Compared to these, another compound was less stable in mouse and human plasma with a t˜_{z} of 238 min and 259 min, respectively (Table 7). All tested compounds showed high levels of human plasma protein binding (PPB >99%), similar to that of warfarin (PPB >99%, see Table 7). Although it was commonly thought that only the unbound drug is available to engage its targets, recent findings have suggested that compounds with >99% PPB remain valid drug candidates without requiring further property optimization.

TABLE 7
Physicochemical Parameters and in vitro ADME Properties of Selected Compounds

| | PBS | liver microsomal stability | | plasma stability | plasma |
|---|---|---|---|---|---|
| | solubility^{[b]} | (human; mouse) | | (human; mouse) | protein |
| compd. | (μM) | CLᵢₙₜ (μL/min/mg) | t_{1/2} (min) | t_{1/2} (min) | binding |
| 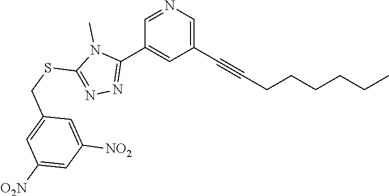 [SMILES: CCCCCCC#Cc1cncc(-c2nnc(SCc3cc([N+](=O)[O-])cc([N+](=O)[O-])c3)n2C)c1] | <1.6 | 357; 287 | 3.9; 4.8 | >480; >480 | >99.9% |
| 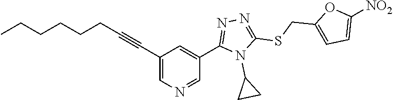 [SMILES: CCCCCCC#Cc1cncc(-c2nnc(SCc3ccc([N+](=O)[O-])o3)n2C2CC2)c1] | 3.1 | 470; 546 | 1.9; 2.5 | 259; 238 | >99.9% |
| 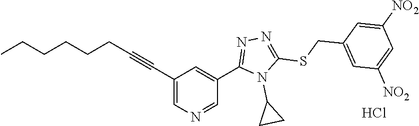 [SMILES: CCCCCCC#Cc1cncc(-c2nnc(SCc3cc([N+](=O)[O-])cc([N+](=O)[O-])c3)n2C2CC2)c1 ; Cl] | 3.1 | 725; 235 | 3.0; 5.9 | >480; >480 | >99.9% |
| verapamil | >500^{[c]} | 62; 257 | 23; 5.4 | — | — |
| reserpine | 7.8^{[d]} | — | — | — | — |
| propantheline | — | — | — | 21; 24 | — |
| warfarin | — | — | — | >480; >480 | >99.9% |

^{[a]}Data collected at Cyprotex.
^{[b]}Phosphate-buffered saline (PBS) solubility limit is the highest concentration with no detectable precipitate.
^{[c]}High solubility control and ^{[d]}low solubility control for PBS solubility. Plasma stability data for compounds in human and mouse plasma (n = 1). Liver microsomal stability data for compounds in human and mouse plasma (n = 1).

1. A compound of Formula Ia or Ib or a pharmaceutically acceptable salt thereof, 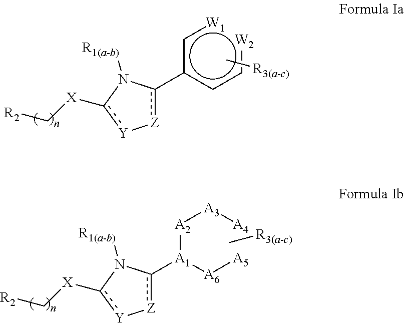
wherein W₁ and W₂ independently comprise N or CR₁₀;
wherein X comprises S, O, NH, NR₄, or CH₂;
wherein Y and Z independently comprise S, O, N, NH, NR₄, CH, or CH₂;
wherein R₄ comprises hydrogen, halogen, hydroxyl, cyano, nitro, carboxyl, (carboxyl ester)amino, (carboxyl ester)oxy, acyl, formyl; or substituted or unsubstituted C1-C10 alkyl, C3-C7 cycloalkyl, C3-C7 heterocycloalkyl, alkenyl, alkoxy, alkynyl, amino, aminosulfinyl, sulfinyl, sulfonyl, substituted sulfinyloxy, sulfonyloxy, aminosulfonyloxy, aminosulfinyloxy, aminosulfonylamino, acylamino, aminocarbonyloxy, aminocarbonylamino, aminothiocarbonylamino, aminocarbonyl, aminocarbonyl, acyloxy, C3-C7 aryl, or C3-C7 heteroaryl;
wherein n is from 0 to 9;
wherein each of R₁ₐ and R₂ independently comprises hydrogen, halogen, hydroxyl, cyano, nitro, carboxyl, (carboxyl ester)amino, (carboxyl ester)oxy, acyl, formyl; or substituted or unsubstituted C1-C10 alkyl, C3-C7 cycloalkyl, C3-C7 heterocycloalkyl, alkenyl, alkoxy, alkynyl, amino, aminosulfinyl, sulfinyl, sulfonyl, substituted sulfinyloxy, sulfonyloxy, aminosulfonyloxy, aminosulfinyloxy, aminosulfonylamino, acylamino, aminocarbonyloxy, aminocarbonylamino, aminothiocarbonylamino, aminocarbonyl, aminocarbonyl, acyloxy, C3-C7 aryl, or C3-C7 heteroaryl;
wherein each of R_{3a-3c} independently comprises hydrogen, halogen, hydroxyl, cyano, nitro, carboxyl, (carboxyl ester)amino, (carboxyl ester)oxy, acyl, formyl; or substituted or unsubstituted C1-C10 alkyl, C3-C7 cycloalkyl, C3-C7 heterocycloalkyl, alkenyl, alkoxy, alkynyl, amino, aminosulfinyl, sulfinyl, sulfonyl, substituted sulfinyloxy, sulfonyloxy, aminosulfonyloxy, aminosulfinyloxy, aminosulfonylamino, acylamino, aminocarbonyloxy, aminocarbonylamino, aminothiocarbonylamino, aminocarbonyl, aminocarbonyl, acyloxy, C3-C7 aryl, or C3-C7 heteroaryl, or wherein one of R_{3a-3c} is an N, NR₁₁, CH, or CH₂ group connected to an R₁₀ by a substituted or unsubstituted C1-C3 alkyl chain;
wherein each R₁₀ independently comprises H or is an N, NR₁₂, CH, or CH₂ group connected to one of R_{3a-3c} by a substituted or unsubstituted C₁-C₃ alkyl chain;
wherein R₁₁ and R₁₂, if present, independently comprise H or C1-C4 alkyl;
wherein R_{1b} is absent or comprises hydrogen, halogen, hydroxyl, cyano, nitro, carboxyl, (carboxyl ester)amino, (carboxyl ester)oxy, acyl, formyl; or substituted or unsubstituted C1-C10 alkyl, C3-C7 cycloalkyl, C3-C7 heterocycloalkyl, alkenyl, alkoxy, alkynyl, amino, aminosulfinyl, sulfinyl, sulfonyl, substituted sulfinyloxy, sulfonyloxy, aminosulfonyloxy, aminosulfinyloxy, aminosulfonylamino, acylamino, aminocarbonyloxy, aminocarbonylamino, aminothiocarbonylamino, aminocarbonyl, aminocarbonyl, acyloxy, C3-C7 aryl, or C3-C7 heteroaryl; and
wherein A₁-A₆ are independently selected from NR₁₄, CR₁₄, C(R₁₄)₂, N, and O, wherein each R₁₄ is independently H or is one of R_{3a-3c}.

2. The compound of claim 1, wherein W₁ is N, W₂ is CR₁₀, and R₁₀ is H.

3. The compound of claim 1, wherein the compound has a formula 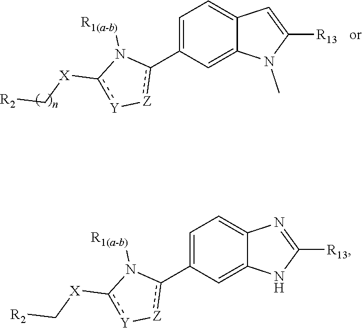
wherein R₁₃ is selected from hydrogen, halogen, hydroxyl, cyano, nitro, carboxyl, (carboxyl ester)amino, (carboxyl ester)oxy, acyl, formyl; or substituted or unsubstituted C1-C10 alkyl, C3-C7 cycloalkyl, C3-C7 heterocycloalkyl, alkenyl, alkoxy, alkynyl, amino, aminosulfinyl, sulfinyl, sulfonyl, substituted sulfinyloxy, sulfonyloxy, aminosulfonyloxy, aminosulfinyloxy, aminosulfonylamino, acylamino, aminocarbonyloxy, aminocarbonylamino, aminothiocarbonylamino, aminocarbonyl, aminocarbonyl, acyloxy, C3-C7 aryl, or C3-C7 heteroaryl.

4.-7. (canceled)

8. The compound of claim 1, wherein R₂ is 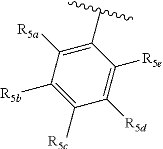
and R_{5a-R}_{₅ₑ} are independently hydrogen, halogen, cyano, or nitro.

9.-10. (canceled)

11. The compound of claim 8, wherein R₅ₐ, R_{5d}, and R₅ₑ, are hydrogen, R_{5b} is chloro, and R_{5c} is chloro or fluoro.

12. The compound of claim 1, wherein R₂ is 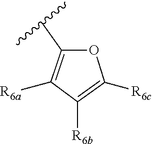
and R₆ₐ-R_{6c} are independently hydrogen or nitro.

13. (canceled)

14. The compound of claim 1, wherein n is 0 or 1.

15. (canceled)

16. The compound of claim 1, wherein X is S.

17. The compound of claim 1, wherein A₁-Aₑ and R_{3a-3c}, if present, form a saturated heterocycle selected from: 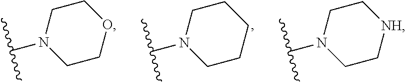 [SMILES: *N1CCOCC1 ; *N1CCCCC1 ; "," ; *N1CCNCC1]
or any combination thereof.

18. The compound of claim 1, wherein the compound is 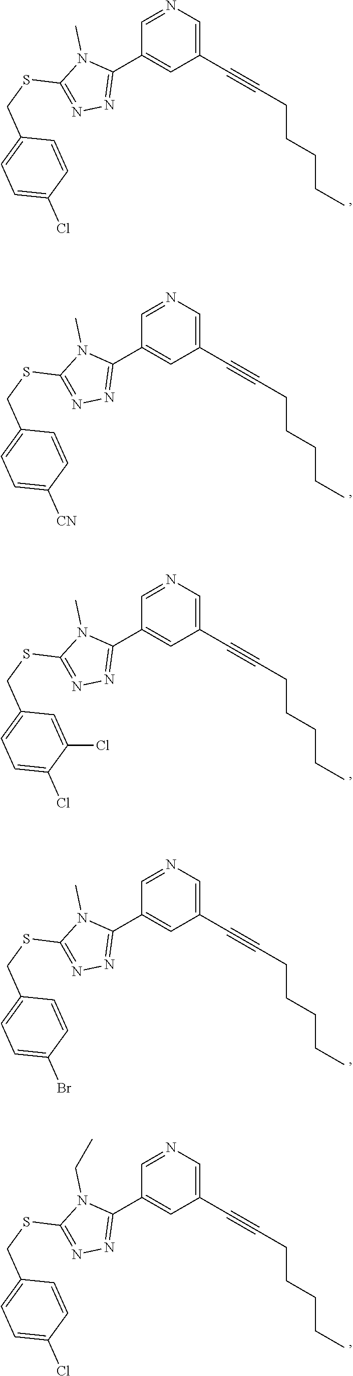 [SMILES: CCCCCC#Cc1cncc(-c2nnc(SCc3ccc(Cl)cc3)n2C)c1 ; CCCCCC#Cc1cncc(-c2nnc(SCc3ccc(C#N)cc3)n2C)c1 ; CCCCCC#Cc1cncc(-c2nnc(SCc3ccc(Cl)c(Cl)c3)n2C)c1 ; CCCCCC#Cc1cncc(-c2nnc(SCc3ccc(Br)cc3)n2C)c1 ; CCCCCC#Cc1cncc(-c2nnc(SCc3ccc(Cl)cc3)n2CC)c1] 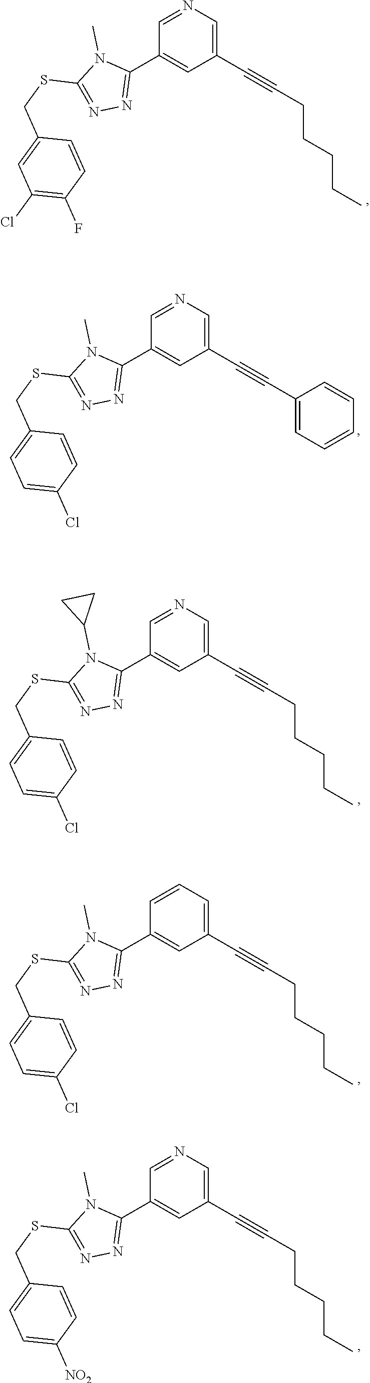 [SMILES: CCCCCC#Cc1cncc(-c2nnc(SCc3ccc(F)c(Cl)c3)n2C)c1 ; Cn1c(SCc2ccc(Cl)cc2)nnc1-c1cncc(C#Cc2ccccc2)c1 ; CCCCCC#Cc1cncc(-c2nnc(SCc3ccc(Cl)cc3)n2C2CC2)c1 ; CCCCCC#Cc1cccc(-c2nnc(SCc3ccc(Cl)cc3)n2C)c1 ; CCCCCC#Cc1cncc(-c2nnc(SCc3ccc([N+](=O)[O-])cc3)n2C)c1] 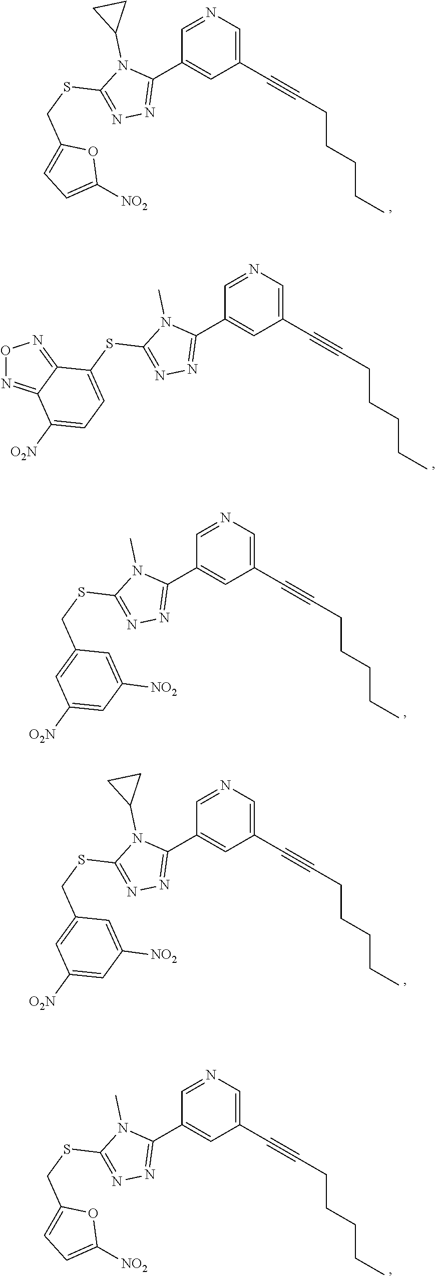 [SMILES: CCCCCC#Cc1cncc(-c2nnc(SCc3ccc([N+](=O)[O-])o3)n2C2CC2)c1 ; CCCCCC#Cc1cncc(-c2nnc(Sc3ccc([N+](=O)[O-])c4nonc34)n2C)c1 ; CCCCCC#Cc1cncc(-c2nnc(SCc3cc([N+](=O)[O-])cc([N+](=O)[O-])c3)n2C)c1 ; CCCCCC#Cc1cncc(-c2nnc(SCc3cc([N+](=O)[O-])cc([N+](=O)[O-])c3)n2C2CC2)c1 ; CCCCCC#Cc1cncc(-c2nnc(SCc3ccc([N+](=O)[O-])o3)n2C)c1] 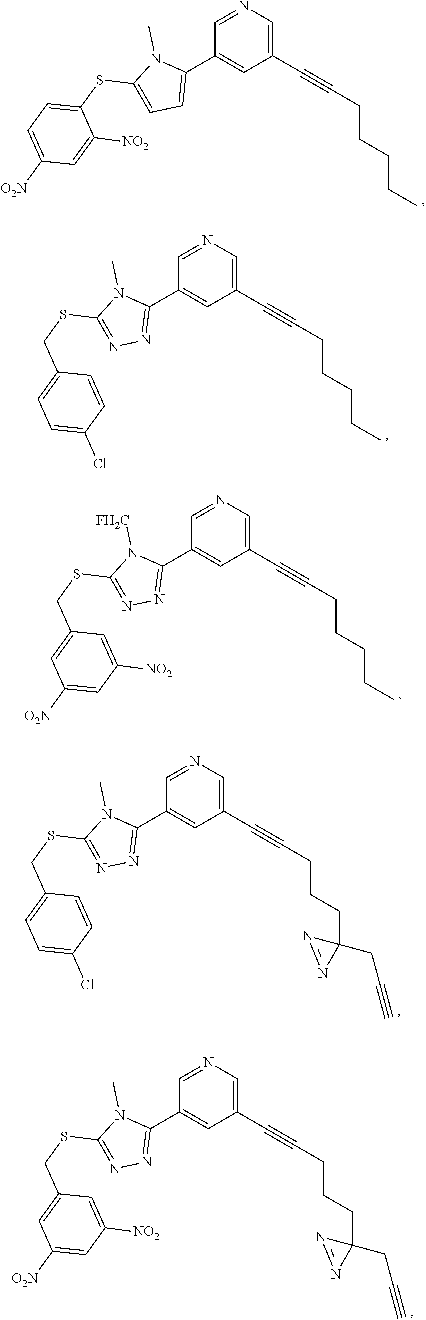 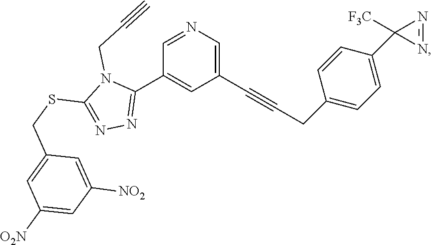 [SMILES: C#CCn1c(SCc2cc([N+](=O)[O-])cc([N+](=O)[O-])c2)nnc1-c1cncc(C#CCc2ccc(C3(C(F)(F)F)N=N3)cc2)c1] 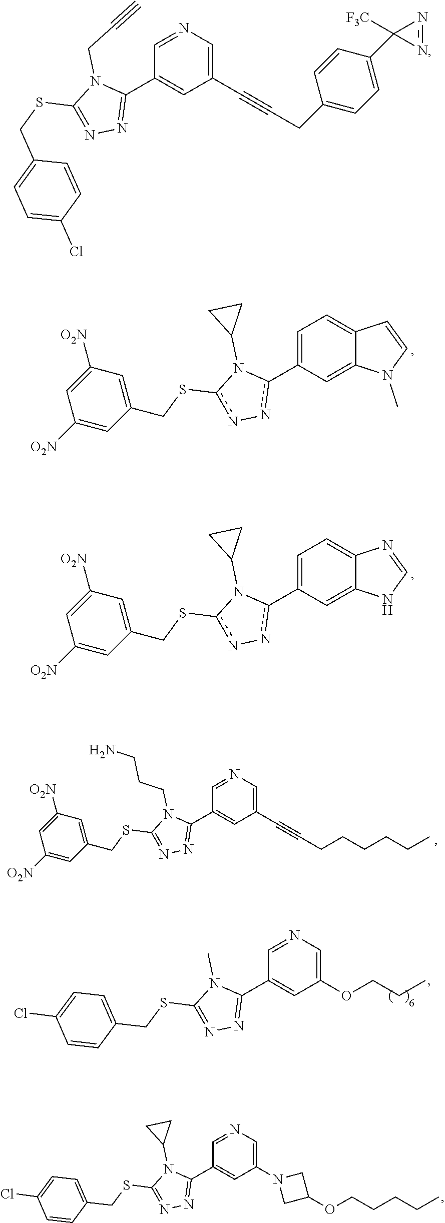 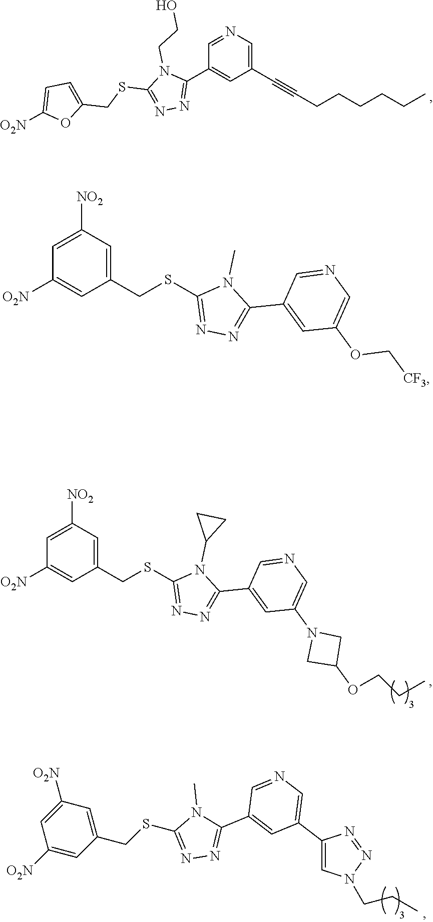
a salt thereof, or any combination thereof.

19.-31. (canceled)

32. The compound of claim 1, wherein the salt is a hydrochloride salt.

33. The compound of claim 1 wherein the compound has an IC₅₀ of less than 1 μM against Mycobacterium tuberculosis.

34. The compound of claim 1, wherein the compound exhibits an MraY inhibition of at least about 45% at a 250 μM concentration.

35. The compound of claim 1, wherein the compound is not cytotoxic.

36. A pharmaceutical composition comprising the compound or pharmaceutically acceptable salt thereof of claim 1.

37. The pharmaceutical composition of claim 36, further comprising at least one excipient.

38. The pharmaceutical composition of claim 36, wherein the pharmaceutical composition is administered to a subject orally, by inhalation, parenterally, intravenously, mucosally, or any combination thereof.

39. A method for treating a bacterial infection, the method comprising administering to a subject a therapeutically effective amount of the compound of claim 1.

40. The method of claim 39, wherein the bacterial infection comprises multidrug-resistant tuberculosis, extensively drug-resistant tuberculosis, or drug-sensitive tuberculosis.

41. The method of claim 39, wherein the subject is a human.
